SEQUENCE LISTING (121 sequences)

SEQ ID NO: 1 - protein, 54 aa, Komagataella phaffii / Komagataella pastoris
KQFRCTDCSRRFRRSEHLKRHHRSVHSNERPFHCAHCDKRFSRSDNLSQHLRTH

SEQ ID NO: 2 - protein, 54 aa, Yarrowia lipolytica
KTFVCTHCQRRFRRQEHLKRHFRSLHTREKPFNCDTCGKKFSRSDNLAQHMRTH

SEQ ID NO: 3 - protein, 54 aa, Trichoderma reesei
KTFVCDLCNRRFRRQEHLKRHYRSLHTQEKPFECNECGKKFSRSDNLAQHARTH

SEQ ID NO: 4 - protein, 53 aa, Schizosaccharomyces pombe
KSFVCPECSKKFKRSEHLRRHIRSLHTSEKPFVCICGKRFSRRDNLRQHERLH

SEQ ID NO: 5 - protein, 54 aa, Saccharomyces cerevisiae
KPFKCKDCEKAFRRSEHLKRHIRSVHSTERPFACMFCEKKFSRSDNLSQHLKTH

SEQ ID NO: 6 - protein, 54 aa, Saccharomyces cerevisiae
KPFHCHICPKSFKRSEHLKRHVRSVHSNERPFACHICDKKFSRSDNLSQHIKTH

SEQ ID NO: 7 - protein, 54 aa, Kluyveromyces lactis
KPFKCDQCNKTFRRSEHLKRHVRSVHSTERPFHCQFCDKKFSRSDNLSQHLKTH

SEQ ID NO: 8 - protein, 54 aa, Kluyveromyces lactis
KPFGCEYCDRRFKRQEHLKRHIRSLHICEKPYGCHLCGKKFSRSDNLSQHLKTH

SEQ ID NO: 9 - protein, 54 aa, Candida boidinii
KPFRCSLCEKSFKRQEHLKRHHRSVHSGEKPHICQTCDKRFSRTDNLAQHLRTH

SEQ ID NO: 10 - protein, 54 aa, Aspergillus niger
KTFVCTLCSRRFRRQEHLKRHYRSLHTQDKPFECNECGKKFSRSDNLAQHARTH

SEQ ID NO: 11 - protein, 54 aa, Saccharomyces cerevisiae
KQFGCEFCDRRFKRQEHLKRHVRSLHMCEKPFTCHICNKNFSRSDNLNQHVKTH

SEQ ID NO: 12 - protein, 57 aa, Artificial sequence
  synMSN4
KQFRCTDCSRRFRRSEHLKRHHRSVHSNERPFHCAHCDKRFSRSDNLSQHLRTHRKQ

SEQ ID NO: 13 - protein, 341 aa, Artificial sequence
  scFv
MRFPSIFTAVLFAASSALAAPVNTTTEDETAQIPAEAVIGYSDLEGDFDVAVLPFSNSTNNGLLFINTTIASIAAKEEGVSLEKRQEQLMESGGGLVTLGGSLKLSCKASGIDFSHYGISWVRQAPGKGLEWIAYIYPNYGSVDYASWVNGRFTISLDNAQNTVFLQMISLTAADTATYFCARDRGYYSGSRGTRLDLWGQGTLVTISSGGGGSGGGGSGGGGSELVMTQTPPSLSASVGETVRIRCLASEFLFNGVSWYQQKPGKPPKFLISGASNLESGVPPRFSGSGSGTDYTLTIGGVQAEDVATYYCLGGYSGSSGLTFGAGTNVEIKGGHHHHHH

SEQ ID NO: 14 - protein, 362 aa, Artificial sequence
  VHH
MRFPSIFTAVLFAASSALAAPVNTTTEDETAQIPAEAVIGYSDLEGDFDVAVLPFSNSTNNGLLFINTTIASIAAKEEGVSLEKRQVQLQESGGGLVQAGGSLRLSCAASGRTFTSFAMGWFRQAPGKEREFVASISRSGTLTRYADSAKGRFTISVDNAKNTVSLQMDNLNPDDTAVYYCAADLHRPYGPGTQRSDEYDSWGQGTQVTVSSGGGSGGGGSGGGGSGGGGSGGGGSGGGEVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGHHHHHH

SEQ ID NO: 15 - protein, 356 aa, Komagataella phaffii
MSTTKPMQVLAPDLTETPKTYSLGVHLGKGKDKLQDPTELYSMILDGMDHSQLNSFINDQLNLGSLRLPANPPAASGAKRGANVSSINMDDLQTFDFNFDYERDSSPLELNMDSQSLMFSSPEKAPCGSLPSQHQPHSQVAAAQGTTINPRQLSTSSASSFVSSDFDVDSLLADEYAEKLEYGAISSASSSICSNSVLPSQGVTSQHSSPIEQRPRVGNSKRLSDFWMQDEAVTAISTWLKAEIPSSLATPAPTVTQISSPSLSTPEPRKKETKQRKRAKSIDTNERSEQVAASNSDDEKQFRCTDCSRRFRRSEHLKRHHRSVHSNERPFHCAHCDKRFSRSDNLSQHLRTHRKQ

SEQ ID NO: 16 - protein, 357 aa, Komagataella pastoris
MSTTKPMQVLAPDLTETPKTYSLGVHLGKGKDKLQDPTELYSMILDGMDHSQLNSFINDQLNLGSLRLPANPPAAGGAKRGANVSSINMDDLQTFDFNFDYERDSSPLELNMDSQTLLFSSPEKAPPCGSLPSQHQPHSQGAAAQGTTINPRQLSTSSASSFVSSDFDVDSLLAEEYAEKLEYGAISSASSSICSNSVLPNQGVTSQHSSPIEQRPRVGNSKRLSDFWMQDEAVTAISTWLKAEIPSSLATPAPTVTKISSPTLSTPEPRKKETKQRKRAKSIDTNERSEQVAASGSDDEKQFRCTDCSRRFRRSEHLKRHHRSVHSNERPFHCAHCDKRFSRSDNLSQHLRTHRKQ

SEQ ID NO: 17 - protein, 285 aa, Yarrowia lipolytica
MDLELEIPVLHSMDSHHQVVDSHRLAQQQFQYQQIHMLQQTLSQQYPHTPSTTPPIYMLSPADYEKDAVSISPVMLWPPSAHSQASYHYEMPSVISPSPSPTRSFCNPRELEVQDELEQLEQQPAALSVEHLFDIENSSIEYAHDELHDTSSCSDSQSSFSPQQSPASPASTYSPLEDEFLNLAGSELKSEPSADDEKDDVDTELPQQPEIIIPVSCRGRKPSIDDSKKTFVCTHCQRRFRRQEHLKRHFRSLHTREKPFNCDTCGKKFSRSDNLAQHMRTHPRD

SEQ ID NO: 18 - protein, 534 aa, Trichoderma reesei
MDGMMSQPMGQQAFYFYNHEHKMSPRQVIFAQQMAAYQMMPSLPPTPMYSRPNSSCSQPPTLYSNGPSVMTPTSTPPLSSRKPMLVDTEFGDNPYFPSTPPLSASGSTVGSPKACDMLQTPMNPMFSGLEGIAIKDSIDATESLVLDWASIASPPLSPVYLQSQTSSGKVPSLTSSPSDMLSTTASCPSLSPSPTPYARSVTSEHDVDFCDPRNLTVSVGSNPTLAPEFTLLADDIKGEPLPTAAQPSFDFNPALPSGLPTFEDFSDLESEADFSSLVNLGEINPVDISRPRACTGSSVVSLGHGSFIGDEDLSFDDEAFHFPSLPSPTSSVDFCDVHQDKRQKKDRKEAKPVMNSAAGGSQSGNEQAGATEAASAASDSNASSASDEPSSSMPAPTNRRGRKQSLTEDPSKTFVCDLCNRRFRRQEHLKRHYRSLHTQEKPFECNECGKKFSRSDNLAQHARTHSGGAIVMNLIEESSEVPAYDGSMMAGPVGDDYSTYGKVLFQIASEIPGSASELSSEEGEQGKKKRKRSD

SEQ ID NO: 19 - protein, 582 aa, Schizosaccharomyces pombe
MVFFPEAMPLVTLSERMVPQVNTSPFAPAQSSSPLPSNSCREYSLPSHPSTHNSSVAYVDSQDNKPPLVSTLHFSLAPSLSPSSAQSHNTALITEPLTSFIGGTSQYPSASFSTSQHPSQVYNDGSTLNSNNTTQQLNNNNGFQPPPQNPGISKSRIAQYHQPSQTYDDTVDSSFYDWYKAGAQHNLAPPQSSHTEASQGYMYSTNTAHDATDIPSSFNFYNTQASTAPNPQEINYQWSHEYRPHTQYQNNLLRAQPNVNCENFPTTVPNYPFQQPSYNPNALVPSYTTLVSQLPPSPCLTVSSGPLSTASSIPSNCSCPSVKSSGPSYHAEQEVNVNSYNGGIPSTSYNDTPQQSVTGSYNSGETMSTYLNQTNTSGRSPNSMEATEQIGTIGTDGSMKRRKRRQPSNRKTSVPRSPGGKSFVCPECSKKFKRSEHLRRHIRSLHTSEKPFVCICGKRFSRRDNLRQHERLHVNASPRLACFFQPSGYYSSGAPGAPVQPQKPIEDLNKIPINQGMDSSQIENTNLMLSSQRPLSQQIVPEIAAYPNSIRPELLSKLPVQTPNQKMPLMNPMHQYQPYPSS

SEQ ID NO: 20 - protein, 630 aa, Saccharomyces cerevisiae
MLVFGPNSSFVRHANKKQEDSSIMNEPNGLMDPVLSTTNVSATSSNDNSANNSISSPEYTFGQFSMDSPHRTDATNTPILTATTNTTANNSLMNLKDTASLATNWKWKNSNNAQFVNDGEKQSSNANGKKNGGDKIYSSVATPQALNDELKNLEQLEKVFSPMNPINDSHFNENIELSPHQHATSPKTNLLEAEPSIYSNLFLDARLPNNANSTTGLNDNDYNLDDTNNDNTNSMQSILEDFVSSEEALKFMPDAGRDARRYSEVVTSSFPSMTDSRNSISHSIEFWNLNHKNSSNSKPTQQIIPEGTATTERRGSTISPTTTINNSNPNFKLLDHDVSQALSGYSMDFSKDSGITKPKSISSSLNRISHSSSTTRQQRASLPLIHDIESFANDSVMANPLSDSASFLSEENEDDAFGALNYNSLDATTMSAFDNNVDPFNILKSSPAQDQQFIKPSMMLSDNASAAAKLATSGVDNITPTPAFQRRSYDISMNSSFKILPTSQAHHAAQHHQQQPTKQATVSPNTRRRKSSSVTLSPTISHNNNNGKVPVQPRKRKSITTIDPNNYDKNKPFKCKDCEKAFRRSEHLKRHIRSVHSTERPFACMFCEKKFSRSDNLSQHLKTHKKHGDF

SEQ ID NO: 21 - protein, 704 aa, Saccharomyces cerevisiae
MTVDHDFNSEDILFPIESMSSIQYVENNNPNNINNDVIPYSLDIKNTVLDSADLNDIQNQETSLNLGLPPLSFDSPLPVTETIPSTTDNSLHLKADSNKNRDARTIENDSEIKSTNNASGSGANQYTTLTSPYPMNDILYNMNNPLQSPSPSSVPQNPTINPPINTASNETNLSPQTSNGNETLISPRAQQHTSIKDNRLSLPNGANSNLFIDTNPNNLNEKLRNQLNSDTNSYSNSISNSNSNSTGNLNSSYFNSLNIDSMLDDYVSSDLLLNDDDDDTNLSRRRFSDVITNQFPSMTNSRNSISHSLDLWNHPKINPSNRNTNLNITTNSTSSSNASPNTTTMNANADSNIAGNPKNNDATIDNELTQILNEYNMNFNDNLGTSTSGKNKSACPSSFDANAMTKINPSQQLQQQLNRVQHKQLTSSHNNSSTNMKSFNSDLYSRRQRASLPIIDDSLSYDLVNKQDEDPKNDMLPNSNLSSSQQFIKPSMILSDNASVIAKVATTGLSNDMPFLTEEGEQNANSTPNFDLSITQMNMAPLSPASSSSTSLATNHFYHHFPQQGHHTMNSKIGSSLRRRKSAVPLMGTVPLTNQQNNISSSSVNSTGNGAGVTKERRPSYRRKSMTPSRRSSVVIESTKELEEKPFHCHICPKSFKRSEHLKRHVRSVHSNERPFACHICDKKFSRSDNLSQHIKTHKKHGDI

SEQ ID NO: 22 - protein, 694 aa, Kluyveromyces lactis
MALGRYESGNRGSYTSENSLDIRNDSVSTNYGDKVATEPTLGYTRRNESTGSTPPAVRNVKRETLQNNMGSTPTELNDFLAMLDDKTTYSEVVQSAEPRLGFEDRQKSTEYHTGSELSGNSNGIALSGSPVDSYPNSQKISNHSSRNNTLNYSPNIEPSVMSVGTLSPQVADISSRKNSTVGNSLNSNSIQEFLNQIDLSHSEEQYINPYLLNKESYSTNNNTNNGHNSFEVTHSDSLFMDSGADAEAEDHGELNQLNENPLLLDDVTVSPNPTSDDRRRMSEVVNGNIAYPAHSRGSISHQVDFWNLGSGNPISSNQNQSSNSQVQQDNNSELFDLMSFKNKGRQHLQQQLQQQQQQAQLQSQMHRQQIQQRQQHQQQQSQQRHSAFKIDNELTQLLNAYNMTQSNLPSNGSNINTNKLRTGSFTQSNVKRSNSSNQEAHNRVGKQRYSMSLLDGNQDVISKLYGDMTRNGLSWENAIISDDEEDPEDHEDALRLRRKSALNRSTQVASQNPTETSSSGRFISPQLLNNDPLLETQISTSQTSLGLDRAGLNFKLNLPITNPEALIGSSQPDVQTLNVYSESNVLPTSAQSTTTKKKRSSMSKSKGPKSTSPMDEEEKPFKCDQCNKTFRRSEHLKRHVRSVHSTERPFHCQFCDKKFSRSDNLSQHLKTHKKHGDITELPPPRRVTNSSNKH

SEQ ID NO: 23 - protein, 474 aa, Kluyveromyces lactis
MNPTMYQNDFVTISQETLRDGTMFNLQLKRTPPADNMDNSNIGANKYNQWQFDYEEQELSNDLTGKTLEDEIFSFQQGTSIRAMGDDIRRLSISEYHRDDPMYYEYEFFNKDVMNGSSSRVGNLGGMGSSRSGSVFSDEDNEFDIDMDQESIFVNVGSKSVNDATQTVPHTTNSMALLLSGLDEDVSMNLDLDDENDGTGNSGVKKLFKLNKMFRNNNNRDLISDDEPQQIFKKKYFWSRKPTVPILRNSEPVSTSHGAGLPHAHAEHAPATVSSHNAEFDDDEMTDVETGNPSMAAAIVNPIKLLATGETKNDSDLITLSSHSTKINSLEPDLILSSNSSIMSAVKKNTTGSRSISSASSSLLSPPPMVQVKKAESLSLAKVISSKDSISTIIKKQQGVPKTRGRKPSPILDASKPFGCEYCDRRFKRQEHLKRHIRSLHICEKPYGCHLCGKKFSRSDNLSQHLKTHTHEDK

SEQ ID NO: 24 - protein, 1008 aa, Candida boidinii
MNTTTTPNSNSSSSSNNSIGMGINTGNSELLSFTQSILSSSTSDVVSDSGTILSDSVSTIKNYNITNNNNNKNNNNNTNTPSPNNNYKLSDTYNYNTNTIPNNTSYNLDPMSNSNSQNTNTTSADDTDLYSAAIGSVSNSNKTITTNNNNNINNNNKLDYEDLNVLINYDLESINCLADQQPRDKDMNIIDLFCDLATSNDNIVTNMADNVSITNTITTNNTSTTNTPTDLNLNPVFQTFPSPSSVNTKQFVHPQSIRKSNKQFSSQYHVQYSPQQQQQQLQQLQFQQLQAQLKIQSQLETHLQQQHQQQSQLQSQQSLENGNFPIFDSFSNDLSKTLPSATTPVLQQQQQQQLQQQHLQQQAHIFTGSTSPGYTPSLLSGSNFSVSSKRSSFSSNSNDSPNPNPYHQLSKLNPSTNNNNTNININQIIANENTSLTTASPDLFSKAYMLDDMDPSQQKYQHQRASSSSSTTITPTLPGTNSSSSFAFTYTDDLDRLRKEAELDHFDTNTAKDAIISNNQKFPSLRYPYLSSIITNKKNYDRTINPREIISDYSVLTAPNSTTSPNDLQSLKNNPLISNFDSNASKLLDNENESVKSLFNQSFAFGEFDQTSNNNSSTTSNNNTTNGNNSFYSGNFTAELRSNSNNTNQLFNAIRKNPDLWNSYNMDNNNNDNAADRSDSNSKPVMVNNKPLISPSLPSSSSVSSVVSSVVPKNADPNCLLTPNTSTSNISSPIPPSQLSTNTSSGSNSQYAVNLQHRKRYSTSSIITDHLTGTTGITAPNTSHPNRIINPRSRSRSRSRHGSFASVSNERPTLALINSNSTNSIVNSNNSSSSIKKLSHGSINSSVTSSSSSSSSSSSSNNSSKKRTKSLEIQSISSVNIRNSLLASLKGNPIDESPFDVENSNSGGGGNSMAGGGITRLRASSGSTSSRRSSSSNTDANSSGIGLDDGFKPFRCSLCEKSFKRQEHLKRHHRSVHSGEKPHICQTCDKRFSRTDNLAQHLRTHRNR

SEQ ID NO: 25 - protein, 612 aa, Aspergillus niger
MDGTYTMAPTSVQGQPSFAYYADSQQRQHFTSHPSDMQSYYGQVQAFQQQPQHCMPEQQTLYTAPLMNMHQMATTNAFRGAMNMTPIASPQPSHLKPTIVVQQGSPALMPLDTRFVGNDYYAFPSTPPLSTAGSSISSPPSTSGTLHTPINDSFFAFEKVEGVKEGCEGDVHAEILANADWARSDSPPLTPVFIHPPSLTASQTSELLSAHSSCPSLSPSPSPVVPTFVAQPQGLPTEQSSSDFCDPRQLTVESSINATPAELPPLPTLSCDDEEPRVVLGSEAVTLPVHETLSPAFTCSSSEDPLSSLPTFDSFSDLDSEDEFVNRLVDFPPSGNAYYLGEKRQRVGTTYPLEEEEFFSEQSFDESDEQDLSQSSLPYLGSHDFTGVQTNINEASEEMGNKKRNNRKSLKRASTSDSETDSISKKSQPSINSRATSTETNASTPQTVQARHNSDAHSSCASEAPAAPVSVNRRGRKQSLTDDPSKTFVCTLCSRRFRRQEHLKRHYRSLHTQDKPFECNECGKKFSRSDNLAQHARTHAGGSVVMGVIDTGNATPPTPYEERDPSTLGNVLYEAANAAATKSTTSESDESSSDSPVADRRAPKKRKRDSDA

SEQ ID NO: 26 - protein, 443 aa, Saccharomyces cerevisiae
MSLYPLQRFESNDTVFSYTLNSKTELFNESRNNDKQHFTLQLIPNANANAKEIDNNNVEIINDLTGNTIVDNCVTTATSSNQLERRLSISDYRTENGNYYEYEFFGRRELNEPLFNNDIVENDDDIDLNNESDVLMVSDDELEVNERFSFLKQQPLDGLNRISSTNNLKNLEIHEFIIDPTENIDDELEDSFTTVPQSKKKVRDYFKLNIFGSSSSSNNNSNSLGCEPIQTENSSSQKMFKNRFFRSRKSTLIKSLPLEQENEVLINSGFDVSSNEESDESDHAIINPLKLVGNNKDISTQSIAKTTNPFKSGSDFKMIEPVSKFSNDSRKDLLAAISEPSSSPSPSAPSPSVQSSSSSHGLVVRKKTGSMQKTRGRKPSLIPDASKQFGCEFCDRRFKRQEHLKRHVRSLHMCEKPFTCHICNKNFSRSDNLNQHVKTHASL

SEQ ID NO: 27 - protein, 144 aa, Artificial sequence
  synMSN4
MGKPIPNPLLGLDSTPKKKRKVGGGGSDALDDFDLDMLGSDALDDFDLDMLGSDALDDFDLDMLGSDALDDFDLDMLGGGGSNSDDEKQFRCTDCSRRFRRSEHLKRHHRSVHSNERPFHCAHCDKRFSRSDNLSQHLRTHRKQ

SEQ ID NO: 28 - protein, 678 aa, Komagataella phaffii
MLSLKPSWLTLAALMYAMLLVVVPFAKPVRADDVESYGTVIGIDLGTTYSCVGVMKSGRVEILANDQGNRITPSYVSFTEDERLVGDAAKNLAASNPKNTIFDIKRLIGMKYDAPEVQRDLKRLPYTVKSKNGQPVVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISNNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDYFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGTPPGGQGFDDDDGDFDYDYDYDHDEL

SEQ ID NO: 29 - protein, 677 aa, Komagataella pastoris
MQSLKPSWLTLAALLYAMLMVVVPFAKPVRADDVESYGTVIGIDLGTTYSCVGVMKSGRVEILANDQGNRITPSYVSFTEDERLVGDAAKNLAASNPKNTIFDIKRLIGMKFDSPEVQRDLKRLPYSVKSKNGQPIVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISDNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDFFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGAPPGQGFDDDDGDFDYDYDYDHDEL

SEQ ID NO: 30 - protein, 670 aa, Yarrowia lipolytica
MKFSMPSWGVVFYALLVCLLPFLSKAGVQADDVDSYGTVIGIDLGTTYSCVGVMKGGRVEILANDQGSRITPSYVAFTEDERLVGDAAKNQAANNPFNTIFDIKRLIGLKYKDESVQRDIKHFPYKVKNKDGKPVVVVETKGEKKTYTPEEISAMILTKMKDIAQDYLGKKVTHAVVTVPAYFNDAQRQATKDAGIIAGLNVLRIVNEPTAAAIAYGLDHTDDEKQIVVYDLGGGTFDVSLLSIESGVFEVLATAGDTHLGGEDFDYRVIKHFVKQYNKKHDVDITKNAKTIGKLKREVEKAKRTLSSQMSTRIEIESFFDGEDFSETLTRAKFEELNIDLFKRTLKPVEQVLKDSGVKKEDVHDIVLVGGSTRIPKVQELLEKFFDGKKASKGINPDEAVAYGAAVQAGVLSGEDGVEDIVLLDVNPLTLGIETTGGVMTKLINRNTNIPTKKSQIFSTAVDNQSTVLIQVFEGERTMSKDNNLLGKFELKGIPPAPRGVPQIEVTFELDANGILRVTAHDKGTGKSETITITNDKGRLSKDEIERMVEEAERFAEEDALIRETIEAKNSLENYAHSLRNQVADKSGLGGKISADDKEALNDAVTETLEWLEANSVSATKEDFEEKKEALSAIAYPITSKIYEGGEGGDESNDGGFYADDDEAPFHDEL

SEQ ID NO: 31 - protein, 664 aa, Trichoderma reesei
MARSRSSLALGLGLLCWITLLFAPLAFVGKANAASDDADNYGTVIGIDLGTTYSCVGVMQKGKVEILVNDQGNRITPSYVAFTDEERLVGDSAKNQAAANPTNTVYDVKRLIGRKFDEKEIQADIKHFPYKVIEKNGKPVVQVQVNGQKKQFTPEEISAMILGKMKEVAESYLGKKVTHAVVTVPAYFNDNQRQATKDAGTIAGLNVLRIVNEPTAAAIAYGLDKTDGERQIIVYDLGGGTFDVSLLSIDNGVFEVLATAGDTHLGGEDFDQRIINYLAKAYNKKNNVDISKDLKAMGKLKREAEKAKRTLSSQMSTRIEIEAFFEGNDFSETLTRAKFEELNMDLFKKTLKPVEQVLKDANVKKSEVDDIVLVGGSTRIPKVQSLIEEYFNGKKASKGINPDEAVAFGAAVQAGVLSGEEGTDDIVLMDVNPLTLGIETTGGVMTKLIPRNTPIPTRKSQIFSTAADNQPVVLIQVFEGERSMTKDNNLLGKFELTGIPPAPRGVPQIEVSFELDANGILKVSAHDKGTGKQESITITNDKGRLTQEEIDRMVAEAEKFAEEDKATRERIEARNGLENYAFSLKNQVNDEEGLGGKIDEEDKETILDAVKEATEWLEENGADATTEDFEEQKEKLSNVAYPITSKMYQGAGGSEDDGDFHDEL

SEQ ID NO: 32 - protein, 682 aa, Saccharomyces cerevisiae
MFFNRLSAGKLLVPLSVVLYALFVVILPLQNSFHSSNVLVRGADDVENYGTVIGIDLGTTYSCVAVMKNGKTEILANEQGNRITPSYVAFTDDERLIGDAAKNQVAANPQNTIFDIKRLIGLKYNDRSVQKDIKHLPFNVVNKDGKPAVEVSVKGEKKVFTPEEISGMILGKMKQIAEDYLGTKVTHAVVTVPAYFNDAQRQATKDAGTIAGLNVLRIVNEPTAAAIAYGLDKSDKEHQIIVYDLGGGTFDVSLLSIENGVFEVQATSGDTHLGGEDFDYKIVRQLIKAFKKKHGIDVSDNNKALAKLKREAEKAKRALSSQMSTRIEIDSFVDGIDLSETLTRAKFEELNLDLFKKTLKPVEKVLQDSGLEKKDVDDIVLVGGSTRIPKVQQLLESYFDGKKASKGINPDEAVAYGAAVQAGVLSGEEGVEDIVLLDVNALTLGIETTGGVMTPLIKRNTAIPTKKSQIFSTAVDNQPTVMIKVYEGERAMSKDNNLLGKFELTGIPPAPRGVPQIEVTFALDANGILKVSATDKGTGKSESITITNDKGRLTQEEIDRMVEEAEKFASEDASIKAKVESRNKLENYAHSLKNQVNGDLGEKLEEEDKETLLDAANDVLEWLDDNFETAIAEDFDEKFESLSKVAYPITSKLYGGADGSGAADYDDEDEDDDGDYFEHDEL

SEQ ID NO: 33 - protein, 679 aa, Kluyveromyces lactis
MFSARKSSVGWLVSSLAVFYVLLAVIMPIALTGSQSSRVVARAAEDHEDYGTVIGIDLGTTYSCVAVMKNGKTEILANEQGNRITPSYVSFTDDERLIGDAAKNQAASNPKNTIFDIKRLIGLQYNDPTVQRDIKHLPYTVVNKGNKPYVEVTVKGEKKEFTPEEVSGMILGKMKQIAEDYLGKKVTHAVVTVPAYFNDAQRQATKDAGAIAGLNILRIVNEPTAAAIAYGLDKTEDEHQIIVYDLGGGTFDVSLLSIENGVFEVQATAGDTHLGGEDFDYKLVRHFAQLFQKKHDLDVTKNDKAMAKLKREAEKAKRSLSSQTSTRIEIDSFFNGIDFSETLTRAKFEELNLALFKKTLKPVEKVLKDSGLQKEDIDDIVLVGGSTRIPKVQQLLEKFFNGKKASKGINPDEAVAYGAAVQAGVLSGEEGVEDIVLLDVNALTLGIETTGGVMTPLIKRNTAIPTKKSQIFSTAVDNQKAVRIQVYEGERAMVKDNNLLGNFELSDIRAAPRGVPQIEVTFALDANGILTVSATDKDTGKSESITIANDKGRLSQDDIDRMVEEAEKYAAEDAKFKAKSEARNTFENFVHYVKNSVNGELAEIMDEDDKETVLDNVNESLEWLEDNSDVAEAEDFEEKMASFKESVEPILAKASASQGSTSGEGFEDEDDDDYFDDEL

SEQ ID NO: 34 - protein, 670 aa, Candida boidinii
MLKFNRSFIASLAILYSLLLIIVPLLSQQAHAEDEHETYGTVIGIDLGTTYSCVGVMKSGKVEILANDQGNRITPSYVAFTDEERLVGDAAKNQAPSNPHNTIFDIKRLIGHSYSDKVVQTEKKHLPYNIIEKQGKPAVEVKFQNELKVFTPEEISSMILGKMKQIAEDYLGKKVTHAVVTVPAYFNDAQRQATKDAGTIAGLNVLRIVNEPTAAAIAYGLDKEGERQIIVYDLGGGTFDVSLLAIENGVFEVLSTSGDTHLGGEDFDFRVVRHFSKIFKKKHNIDISDNAKAISKLKREVEKAKRTLSTQMSTRIEIDSFVDGIDFSETLSRAKFEEINIELFKKTLKPVQQVLDDAGLKAAEIDDIVLVGGSTRIPKVQEILENFFSGKKATKGINPDEAVAYGAAVQAGILSGSEGASDVVLIDVNPLTLGIETTGNVMTTLIKRNTPIPTKKTQVFSTAVDNQDTVLIKVYEGERAMSTDNNLLGSFELKGIPPAPKGSPQIEVTFSLDVNGILRVSATDKSTGKSNSITISNDHGRLSKEEIDKMVEDGEKYAEQDKLFREKIEAKNDLEKYALGLKTQLADESVAEKLAEDEIETVLDAVKEALEFIDENEDATTEDYSEQKEKLIKIASPITTKLFMQPQGGESADEDDEDFDDDYDYGHDEL

SEQ ID NO: 35 - protein, 672 aa, Aspergillus niger
MARISHQGAAKPFTAWTTIFYLLLVFIAPLAFFGTAHAQDETSPQESYGTVIGIDLGTTYSCVGVMQNGKVEILVNDQGNRITPSYVAFTDEERLVGDAAKNQYAANPRRTIFDIKRLIGRKFDDKDVQKDAKHFPYKVVNKDGKPHVKVDVNQTPKTLTPEEVSAMVLGKMKEIAEGYLGKKVTHAVVTVPAYFNDAQRQATKDAGTIAGLNVLRVVNEPTAAAIAYGLDKTGDERQVIVYDLGGGTFDVSLLSIDNGVFEVLATAGDTHLGGEDFDQRVMDHFVKLYNKKNNVDVTKDLKAMGKLKREVEKAKRTLSSQMSTRIEIEAFHNGEDFSETLTRAKFEELNMDLFKKTLKPVEQVLKDAKVKKSEVDDIVLVGGSTRIPKVQALLEEFFGGKKASKGINPDEAVAFGAAVQGGVLSGEEGTGDVVLMDVNPLTLGIETTGGVMTKLIPRNTVIPTRKSQIFSTAADNQPTVLIQVYEGERSLTKDNNLLGKFELTGIPPAPRGVPQIEVSFDLDANGILKVHASDKGTGKAESITITNDKGRLSQEEIDRMVAEAEEFAEEDKAIKAKIEARNTLENYAFSLKNQVNDENGLGGQIDEDDKQTILDAVKEVTEWLEDNAATATTEDFEEQKEQLSNVAYPITSKLYGSAPADEDDEPSGHDEL

SEQ ID NO: 36 - protein, 665 aa, Ogataea polymorpha
MLTFNKSVVSCAAIIYALLLVVLPLTTQQFVKAESNENYGTVIGIDLGTTYSCVGVMKAGRVEIIPNDQGNRITPSYVAFTEDERLVGDAAKNQIASNPTNTIFDIKRLIGHRFDDKVIQKEIKHLPYKVKDQDGRPVVEAKVNGELKTFTAEEISAMILGKMKQIAEDYLGKKVTHAVVTVPAYFNDAQRQATKDAGTIAGLEVLRIVNEPTAAAIAYGLDKTDEEKHIIVYDLGGGTFDVSLLTIAGGAFEVLATAGDTHLGGEDFDYRVVRHFIKVFKKKHGIDISDNSKALAKLKREVEKAKRTLSSQMSTRIEIDSFVDGIDFSESLSRAKFEELNMDLFKKTLKPVQQVLDDAKMKPDEIDDVVFVGGSTRIPKVQELIENFFNGKKISKGINPDEAVAFGAAVQGGVLSGEEGVEDIVLIDVNPLTLGIETSGGVMTTLIKRNTPIPTQKSQIFSTAADNQPVVLIQVYEGERAMAKDNNLLGKFELTGIPPAPRGVPQIEVTFTLDSNGILKVSATDKGTGKSNSITITNDKGRLSKEEIEKKIEEAEKFAQQDKELREKVESRNALENYAHSLKNQANDENGFGAKLEEDDKETLLDAINEALEFLEDNFDTATKDEFDEQKEKLSKVAYPITSKLYDAPPTSDEEDEDDWDHDEL

SEQ ID NO: 37 - protein, 894 aa, Komagataella phaffii
MRTQKIVTVLCLLLNTVLGALLGIDYGQEFTKAVLVAPGVPFEVILTPDSKRKDNSMMAIKENSKGEIERYYGSSASSVCIRNPETCLNHLKSLIGVSIDDVSTIDYKKYHSGAEMVPSKNNRNTVAFKLGSSVYPVEEILAMSLDDIKSRAEDHLKHAVPGSYSVISDAVITVPTFFTQSQRLALKDAAEISGLKVVGLVDDGISVAVNYASSRQFNGDKQYHMIYDMGAGSLQATLVSISSSDDGGIVIDVEAIAYDKSLGGQLFTQSVYDILLQKFLSEHPSFSESDFNKNSKSMSKLWQAAEKAKTILSANTDTRVSVESLYNDIDFRATIARDEFEDYNAEHVHRITAPIIEALSHPLNGNLTSPFPLTSLSSVILTGGSTRVPMVKKHLESLLGSELIAKNVNADESAVFGSTLRGVTLSQMFKAKQMTVNERSVYDYCLKVGSSEINVFPVGTPLATKKVVELENVDSENQLTIGLYENGQLFASHEVTDLKKSIKSLTQEGKECSNINYEATVELSESRLLSLTRLQAKCADEAEYLPPVDTESEDTKSENSTTSETIEKPNKKLFYPVTIPTQLKSVHVKPMGSSTKVSSSLKIKELNKKDAVKRSIEELKNQLESKLYRVRSYLEDEEVVEKGPASQVEALSTLVAENLEWLDYDSDDASAKDIREKLNSVSDSVAFIKSYIDLNDVTFDNNLFTTIYNTTLNSMQNVQELMLNMSEDALSLMQQYEKEGLDFAKESQKIKIKSPPLSDKELDNLFNTVTEKLEHVRMLTEKDTISDLPREELFKLYQELQNYSSRFEAIMASLEDVHSQRINRLTDKLRKHIERVSNEALKAALKEAKRQQEEEKSHEQNEGEEQSSASTSHTNEDIEEPSESPKVQTSHDEL

SEQ ID NO: 38 - protein, 880 aa, Komagataella pastoris
MKTQKIVTLLCLLLSNVLGALLGIDYGQEFTKAVLVAPGVPFEVILTPDSKRKDNSMMAIKENFKGEIERYYGSAASSVCIRNPEACLNHLKSLIGVPIDDVSTIEYKKYHSGAELVPSKNNRNTVAFNLGSSVYPVEEILAMSLDDIKSRAEDHLKHAVPGSYSVINDAVITVPTFFTQSQRLALKDAAEISGLKVVGLVDDGISVAVNYASSRQFDGNKQYHMIYDMGAGSLQATLVSISSNEDGGIFIDVEAIAYDNSLGGQLFTQSVYDILLQKFLSEHPSFSESDFNKNSKSMSKLWQSAEKAKTILSANTDTRVSVESLYNDIDFRTTITRDEFEDYNAEHVHRITAPIIEALSHPLNENLTSPFPLTSLSSVILTGGSTRVPMVKKHLESLLGSELIAKNVNADESAVFGSTLRGVTLSQMFKARQMTVNERSVYDYCVKVGSSEINVFPVGTPLDTKKVVELENVDNGNQLTVGLYENGHLFANQEVSDLKKSIKSLTQEGKECSNIIYEATFELSESRLFSLTRLQAKCADKVESLPPVDTESDDAKSENSTSSENTEKSNKKLFYPVTIPTQLKFVHVKPMGSSTKISSSLKIKELNKKDAVKRSIEELKNQLESKLYRVRSYLEDEQVVQKGPASQVEALSTQVAENLEWLDYDSDDASAKDIRDKLNFVSESVSFIKNYIDLSDVTLDNNLFTMIYNTTSNSMQNVQELMLNMSEDALSLMQQYEKEGLDFAKESQKIKIKSPPLSDKELDGLFNVVTEKLEYVRTLTEEDGIVGLPREELFKLYQELQNYSSRFEEIMTSLKDVHSQRINRLTDKLNKHIERVNNEALKAALKEAKRQQEEEKSHEQNDEEEQGSSSTSHTKAETEE

SEQ ID NO: 39 - protein, 1007 aa, Yarrowia lipolytica
MKVAHIIQLAAMVATALAAVLAIDYGQEYTKAALLSPGINFEIVLTQDSKRKQPSAIGFKGKADSKFGLERVYGSPAVLMEPRFPSDVVLYHKRLLGGRPKLDNPNYKEYTQMRPACMAVPSNSSRSAIAFQVKDSEWSAEELLAMQISDIKSRADDMLKTQSKSNTDTVKDVVMTVPPHFTHSQRLALADAVDLAGLKLIALVSDGTATAVNYVSTRKFTDEKEYHVVYDMGAGSASATLFSVQDVNGTPVIDIEGVGYDEALAGQDMTNMMVKILAASFMEQNKDKVQLQTFIRDVKAAAKLWKEAERAKAILSANQEVSVSIEAVHNGIDFKTTVTRDDYVRSIEKISTRLNGPLEKALAGFADSPVALKDVKSVILTGGVTRTPVIQEKLKELLGDVPISKNVNTDESIVLGSLLRGVGISSIFKSRDIKVIDRTPHEFDLRLDVLGAKDEILRSEKANVFSKGAAQGESVVSKLDISEIGNANLYLLEDGDSFVRLDVRDMDAIKKELNCEKSAELHVPFDLTLSGTIKVGKAKVVCKGGDAEADAEVTVDDPVEDVVVEEEVVEGETVEGDAKAAKDSKDSKDSKKASKKVDTSRYVPHKTRFVGTKPLTSAAKLKISGHLRSLARKDAERLATSDAANKLESTIYHIKHLIEDAVDQDKVADIKKKIEDAAAWFEEDGLTAGIQELTEKLSVVQPLEDFFKTAGEAIADKATAAASAAGEFVDQAAAAAGVKAGEAADAAKGAADAAGKKAKKAKKAAGKAASQAEEDVLDQLKDANDLIKNIAQLARESGNDVPSEEDIEREMKRAAEGGDSSDSADLSGHLETLMGLQDMLNELNGGEAPSAPGLDVTAIAGITRTIQRLSDKLTELGTPPKDEDDMFRMLGIDPQTFHKFSEEAFEDQASPADQLMDSIGFLQQVLAQDESPDPAALEKMRANIAERQERIAKVAEVAERNQKRQIAALENMLKNAEKTIDISIYNLKQQAPKTASVEDKKAEHDEL

SEQ ID NO: 40 - protein, 985 aa, Trichoderma reesei
RKSPLLKLLGAAFLFSTNVLAISAVLGVDLGTEYIKAALVKPGIPLEIVLTKDSRRKETSAVAFKPAKGALPEGQYPERSYGADAMALAARFPGEVYPNLKPLLGLPVGDAIVQEYAARHPALKLQAHPTRGTAAFKTETLSPEEEAWMVEELLAMELQSIQKNAEVTAGGDSSIRSIVLTVPPFYTIEEKRALQMAAELAGFKVLSLVSDGLAVGLNYATSRQFPNINEGAKPEYHLVFDMGAGSTTATVMRFQSRTVKDVGKFNKTVQEIQVLGSGWDRTLGGDSLNSLIIDDMIAQFVESKGAQKISATAEQVQSHGRAVAKLSKEAERLRHVLSANQNTQASFEGLYEDVDFKYKISRADFETMAKAHVERVNAAIKDALKAANLEIGDLTSVILHGGATRTPFVREAIEKALGSGDKIRTNVNSDEAAVFGAAFRAAELSPSFRVKEIRISEGANYAAGITWKAANGKVHRQRLWTAPSPLGGPAKEITFTEQEDFTGLFYQQVDTEDKPVKSFSTKNLTASVAALKEKYPTCADTGVQFKAAAKLRTENGEVAIVKAFVECEAEVVEKEGFVDGVKNLFGFGKKDQKPLAEGGDKDSADASADSEAETEEASSATKSSSSTSTTKSGDAAESTEAAKEVKKKQLVSIPVEVTLEKAGIPQLTKAEWTKAKDRLKAFAASDKARLQREEALNQLEAFTYKVRDLVDNEAFISASTEAERQTLSEKASEASDWLYEEGDSATKDDFVAKLKALQDLVAPIQNRLDEAEKRPGLISDLRNILNTTNVFIDTVRGQIAAYDEWKSTASAKSAESATSSAAAEATTNDFEGLEDEDDSPKEAEEKPVPEKVVPPLHNSEEIDTLEVLYKETLEWLNKLERQQADVPLTEEPVLVVSELVARRDALDKASLDLALKSYTQYQKNKPKKPTKSKKAKKQDKTKSADKAGPTFEFPEGSVPLSGEELEELVKKYMKEEEETRRQAEG

SEQ ID NO: 41 - protein, 848 aa, Schizosaccharomyces pombe
MKRSVLTIILFFSCQFWHAFASSVLAIDYGTEWTKAALIKPGIPLEIVLTKDTRRKEQSAVAFKGNERIFGVDASNLATRFPAHSIRNVKELLDTAGLESVLVQKYQSSYPAIQLVENEETTSGISFVISDEENYSLEEIIAMTMEHYISLAEEMAHEKITDLVLTVPPHFNELQRSILLEAARILNKHVLALIDDNVAVAIEYSLSRSFSTDPTYNIIYDSGSGSTSATVISFDTVEGSSLGKKQNITRIRALASGFTLKLSGNEINRKLIGFMKNSFYQKHGIDLSHNHRALARLEKEALRVKHILSANSEAIASIEELADGIDFRLKITRSVLESLCKDMEDAAVEPINKALKKANLTFSEINSIILFGGASRIPFIQSTLADYVSSDKISKNVNADEASVKGAAFYGASLTKSFRVKPLIVQDIINYPYLLSLGTSEYIVALPDSTPYGMQHNVTIHNVSTIGKHPSFPLSNNGELIGEFTLSNITDVEKVCACSNKNIQISFSSDRTKGILVPLSAIMTCEHGELSSKHKLGDRVKSLFGSHDESGLRNNESYPIGFTYKKYGEMSDNALRLASAKLERRLQIDKSKAAHDNALNELETLLYRAQAMVDDDEFLEFANPEETKILKNDSVESYDWLIEYGSQSPTSEVTDRYKKLDDTLKSISFRFDQAKQFNTSLENFKNALERAESLLTNFDVPDYPLNVYDEKDVKRVNSLRGTSYKKLGNQYYNDTQWLKDNLDSHLSHTLSEDPLIKVEELEEKAKRLQELTYEYLRRSLQQPKLKAKKGASSSSTAESKVEDETFTNDIEPTTALNSTSTQETEKSRASVTQRPSSLQQEIDDSDEL

SEQ ID NO: 42 - protein, 881 aa, Saccharomyces cerevisiae
MRNVLRLLFLTAFVAIGSLAAVLGVDYGQQNIKAIVVSPQAPLELVLTPEAKRKEISGLSIKRLPGYGKDDPNGIERIYGSAVGSLATRFPQNTLLHLKPLLGKSLEDETTVTLYSKQHPGLEMVSTNRSTIAFLVDNVEYPLEELVAMNVQEIANRANSLLKDRDARTEDFVNKMSFTIPDFFDQHQRKALLDASSITTGIEETYLVSEGMSVAVNFVLKQRQFPPGEQQHYIVYDMGSGSIKASMFSILQPEDTTQPVTIEFEGYGYNPHLGGAKFTMDIGSLIENKFLETHPAIRTDELHANPKALAKINQAAEKAKLILSANSEASINIESLINDIDFRTSITRQEFEEFIADSLLDIVKPINDAVTKQFGGYGTNLPEINGVILAGGSSRIPIVQDQLIKLVSEEKVLRNVNADESAVNGVVMRGIKLSNSFKTKPLNVVDRSVNTYSFKLSNESELYDVFTRGSAYPNKTSILTNTTDSIPNNFTIDLFENGKLFETITVNSGAIKNSYSSDKCSSGVAYNITFDLSSDRLFSIQEVNCICQSENDIGNSKQIKNKGSRLAFTSEDVEIKRLSPSERSRLHEHIKLLDKQDKERFQFQENLNVLESNLYDARNLLMDDEVMQNGPKSQVEELSEMVKVYLDWLEDASFDTDPEDIVSRIREIGILKKKIELYMDSAKEPLNSQQFKGMLEEGHKLLQAIETHKNTVEEFLSQFETEFADTIDNVREEFKKIKQPAYVSKALSTWEETLTSFKNSISEIEKFLAKNLFGEDLREHLFEIKLQFDMYRTKLEEKLRLIKSGDESRLNEIKKLHLRNFRLQKRKEEKLKRKLEQEKSRNNNETESTVINSADDKTTIVNDKTTESNPSSEEDILHDEL

SEQ ID NO: 43 - protein, 863 aa, Kluyveromyces lactis
MRIVFWFLLAIQSLTTCFAAVVGLDFGTHYVKEMVVSLKAPLEIVLNPESKRKDASALAIRSWDSQNYLERFYGSSAVALATRFPSTTFMHLKSLLGKHYEDNLFYYHREHPGLEFVNDASRNAIAFEIDTNTTLSVEELVSMNLKQYMERANQLLKESDDSDNVKSVAIAIPEYFSQEQRAALLDATYLAGIGQTYLCNDAIAVAIDYASKQKSFPAGKPNYHVIYDMGAGSTTASLISILQPENITLPLRIEFLGYGHTESLSGSVLSLAIVDLLENDFLESNPNIRTEQFESDASAKAKLVQAAEKAKLVLSANSDASISIESLYHDLDFKTTITRAKFEEFVAELQSVVIEPILSTLESPLNGKALNVKDLDSVILTGGSTRVPFVKKQLENHLGASLISKNVNSDESAVNGAAIRGVQLSKEFKTRPMKVIDRTTHSFGFSIQNTNISKLVFDAGSEYPKEINLQLPGMELKDTVLKIDLTEDERVFKTIFADVDSKLQSSSLSNCSTAVTYNVTLSLNTDQVFDVQSVVASCLTHEEVPTGTEKEHKRTVSEHIQKHPIPHTVEFTCVKPLSNTEKKERFNKLHKWDQKDKLLLERQRLLNDLEASLYAARELVEDAKELETPPTSYIQQLENMITQYLEFVDDPSSLRTKNIKTMKSNLAELQQRLEIYMDRDNKQLDVEGFRALFDKGEKYLELLSKIQQKSLSELSPLNKNFESLGLNVSEEYTKVKPPKSKTVPFEILNGTIDLLHSQLKHIRDIIEDNNSTYAIEDLFEQKLEVDSLYEKIELLVKKIRAEHKYRLKLLQSVYDRRLTAQKREQEIAKEAQQADGENNDSIKTMEEESIEEHEDANFEQDEL

SEQ ID NO: 44 - protein, 903 aa, Candida boidinii
MKLFNQIICILAIISPILASILGIDFGQQFTKSALLGPGVNFEILLTVDSKRKDISGLAMAIAPNSNNEIQRSFGSSSLSTCVKNPQACFTSFKSLLGKAIDDESTTQLYLKSHPGIELAPANYSRNTIDFKYNHDSYPVEEILAMYFRDIKSRADDYLGDHASPGYTKVQKTAITVPGFFNQAQRRAILDAAEIAGLDVVSLVDDGIAIAAEYASSRAFEIEKEYHLIYDMGAGSTKATLVSFSQNNSDISIVNEGYGFDETLGGELLTNSIKELLISKFLAANPKVKISDFLSNSRAITRLLQSAEKAKSVLSANTETRVSIENIYNEIDFKTTITRAEYEEINSPIMERITAPILKAIQSNSERRDSEDEDQPEITLKDIKSVILAGGSTRVPFVQRHLISLVGEDVISKNVNADEAAVLGTTLRGVQISGLFRSKRMTVVESTTNDFCYKIVSNELDEKDSNLVTVFPVNAKINSKKSVKLNQLKDTFSDFELDFYSNGEFISQANISPSEKFDNKLCTNGTSYIARLELDNSGLASLTSVDQFCYFEKITKLANNSTETDETDKTSSKTSEEEAATTSIASKKEKLEPKIKYPYIRPMGVSTKKICKNRISKLDTKDAVRIEKATTVNKLEAILYSLRSHLDEDEIAEFVNSKSTFIDDISTFVKENLEWLEETYQLPDLEVIQSKLEAATKKVSDIKEFTRVHKSLRDSEFYKNMTTISNEAMFGIQDFLLTMSEDLTSIHTNYTMAGVDINEANKKIEVMTNPFDEATIKEHFDALGELLDKIKTLTEDEDVLAEKSIDYLFQLFKDVVKELEVLTKIKNVLVRIHTKRITKLQEYLVKQLKKKLKAERKSKSKASSKSAKSEEEVTTTSIAPENTDSSNASDSSSDSSTVQKDEL

SEQ ID NO: 45 - protein, 1000 aa, Aspergillus niger
MAPGSQRRPYASLTSLPVLSLILPFLLFVLSFPAPAAAAGSAVLGIDVGTEYLKATLVKPGIPLEIVLTKDSKRKESAAVAFKPTREADASFPERFYGGDALALAARYPDDVYSNLKTLLGLPFDADNELIKSFHSRYPALRLEEAPGDRGTVGLRSNRLGEAERKDAFLIEEILAMQLKQIKANADTLAGKGSDITDAVITYPSFYTAAEKRSLELAAELAGLNVDAFISDNLAVGLNYATSRTFPSVSDGQRPEYHIVYDMGAGSTTASVLRFQSRSVKDVGRFNKTVQEVQVLGTGWDKTLGGDALNDLIVQDMIASLVEEKKLKDRVSPADVQAHGKTMARLWKDAEKARQVLSANTETGASFESLYEEDLNFKYRVTRAKFEELAEQHIARVGKPLEQALEAAGLQLSDIDSVILHGGAIRTPFVQKELERVCGSANKIRTSVNADEAAVFGAAFKGAALSPSFRVKDIRASDASSYAVVLKWDSESKERKQKLFTPTSQVGPEKQVTVKNLDDFEFSFYHQIPVDGNVVESPILGVKTQNLTASVAKLKEDFGCTAANITTKFAIRLSPVDGLPEVASGTVSCEVESAKKGSVVEGVKGFFGLGNKDEQVPLGEEGEPSESITLEPEEPQAATTSSADDATSTTSAKESKKSTPATKLESISISFTSSPLGIPAPTEAELARIKSRLAAFDASDRERALREEALNELESFIYRSRDLVDDEEFAKVVKPEQLTTLQERASEASDWLYGDGDDAKTADFRAKLKSLREIVDPALKRKKENAERPARVELLQQVLKNAKSVIDVMEQQIQQDEDLYSSVTASSSSSSTATESSTSSSTTTGSSSSVDLDEDPYATTSTSSTTKTASATTTPKPSGPKYSIFQPYDLTSLSKTYESTNTWFETQLALQEQLTMTDDPALPVAELDTRLKELERVLNRIYDKMGAAAAKSGKEQSKKNNNNNGKSSKKEKAKAQEEQKKPAKEEEQKDDKKANRKDEL

SEQ ID NO: 46 - protein, 798 aa, Ogataea polymorpha
MKVLGLVALIFIIVQGWASLLAIDFGQDYSKAALVAPGVAFDLVLTDEAKRKHQSGVAISAKDGEIERKFNSHALSACTRSPQSCFFELKSLIGRQIDEPQVTRFEKKYRGVKIVPASSQRRTVAFDVDGQVYLLEEVLGMVLEEIKKRAELHWDQTLGGGSSNTISDVVLSVPDFLDQAQRTALVDAAEIAGLNVVALIDDGIAVALNYASTRDFEQKQYHVIYDVGAGSTKATLVSFSKDNETLRVENEGYGYDETFGGNLFTESLQAIIEDKFLAQTKIKPETLWSDARAMNRLWQSAEKAKLVLSANSETKVSVESLINDIDLKVVVSRDEFEEYMTEHMDRIVAPLAAAMGDRKVESVILAGGSTRVPFVQKHLVKYLGGDELLSKNVNADEAAVFGTLLGGISVSGKFRTRPIELVQHASRNFELAAGGHMTVVFNETTASREAVVALPGLKDTFGEVQVDLFEAGQLFAQYKFKNELNSTVCPNGVEYLANCTLDPRKLFLLHSLEAVCAGDGAVRSSLTAKPLHPGYKPLGSLAKYQSASKLRSLTNQDKQRQQRDALINSLEASLYDLRSYTEDENVVANGPSSMVRAAREMVSELLEWLEDVPAKATVKDIQEKYDDVRVMRIKLETLVNHGDRLLSLAEFTRLKEKALETMYKLQDFMVVMSQDALSLKANFTELGLDFEEANRRVKVKVPEVDEQELEQRMKRISDFVGVVDHFETHKDEIETKDRETLFELRETVLEELKQVQSTYRALKQAHEKRVRGLKEQLKKADKKADKTQEAEPSGHDEL

SEQ ID NO: 47 - protein, 372 aa, Komagataella phaffii
MKVTLSVLAIASQLVRIVCSEGENICIGDQCYPKNFEPDKEWKPVQEGQIIPPGSHVRMDFNTHQREAKLVEENEDIDPSSLGVAVVDSTGSFADDQSLEKIEGLSMEQLDEKLEELIELSHDYEYGSDIILSDQYIFGVAGLVPTKTKFTSELKEKALRIVGSCLRNNADAVEKLLGTVPNTITIQFMSNLVGKVNSTGENVDSVEQKRILSIIGAVIPFKIGKVLFEACSGTQKLLLSLDKLESSVQLRGYQMLDDFIHHPEEELLSSLTAKERLVKHIELIQSFFASGKHSLDIAINRELFTRLIALRTNLESANPNLCKPSTDFLNWLIDEIEATKDTDPHFSKELKHLRFELFGNPLASRKGFSDEL

SEQ ID NO: 48 - protein, 379 aa, Komagataella pastoris
MPKTLSSMKVSLSVLAIATQLVRIVCSEEENICIGDQCYPKNFEPDKEWKPVQEGQIIPPGSHVRMDFNTHQREAKLVDENDDIDSSLMGVAVVDATDTFADDHSLEKIIGLSVSQLDEKLEELVELSHDYEYGSDIILNDQYIIGVAGLVPTKTQFASELKEKALRIVGSCLRNNADAVEKLLGTVPNTITIEFISNLVGKVNTTEENVDPVEQKRILSIIGAIIPFNIGKVLFEACFGTQKLLLSLDKLDDSVQLKAYQVLDDFIHHPQEELLSSLTEKERLVKHIELIQSFFASGKHSLHEAINRELFSRLVALRSDLESTSTNLCTPSTDFLNWLIDEIEATKEVNPHFSQELKHLRFEFFGNPLASRKGFSDEL

SEQ ID NO: 49 - protein, 426 aa, Yarrowia lipolytica
MKFSKTLLLALVAGALAKGEDEICRVEKNSGKEICYPKVFVPTEEWQVVWPDQVIPAGLHVRMDYENGVKEAKINDPNEEVEGVAVAVGEEVPEGEVVIEDLTEENGDEGISANEKVQRAIEKAIKEKRIKEGHKPNPNIPESDHQTFSDAVAALRDYKVNGQAAMLPIALSQLEELSHEIDFGIALSDVDPLNALLQILEDAKVDVESKIMAARTIGASLRNNPHALDKVINSKVDLVKSLLDDLAQSSKEKADKLSSSLVYALSAVLKTPETVTRFVDLHGGDTLRQLYETGSDDVKGRVSTLIEDVLATPDLHNDFSSITGAVKKRSANWWEDELKEWSGVFQRSLPSKLSSKVKSKVYTSLAAIRRNFRESVDVSEEFLEWLDHPKKAAAEIGDDLVKLIKQDRGELWGNAKARKYDARDEL

SEQ ID NO: 50 - protein, 406 aa, Trichoderma reesei
MRPLALIFALILGLLLCLAAPATASSSSSQHSPQAASDESDLICHTSNPDECYPRVFVPTHEFQPVHDDQQLPNGLHVRLNIWTGQKEAKINVPDEANPDLDGLPVDQAVVLVDQEQPEIIQIPKGAPKYDNVGKIKEPAQEGDAQTEAIAFAETFNMLKTGKSPSAEEFDNGLEGLEELSHDIYYGLKITEDADVVKALFCLMGARDGDASEGATPRDQQAAAILAGALSNNPSALAEIAKIWPELLDSSCPRDGATISDRFYQDTVSVADSPAKVKAAVSAINGLIKDGAIRKQFLENSGMKQLLSVLCQEKPEWAGAQRKVAQLVLDTFLDEDMGAQLGQWPRGKASNNGVCAAPETALDDGCWDYHADRMVKLHGTPWSKELKQRLGDARKANSKLPDHGEL

SEQ ID NO: 51 - protein, 421 aa, Saccharomyces cerevisiae
MVRILPIILSALSSKLVASTILHSSIHSVPSGGEIISAEDLKELEISGNSICVDNRCYPKIFEPRHDWQPILPGQELPGGLDIRINMDTGLKEAKLNDEKNVGDNGSHELIVSSEDMKASPGDYEFSSDFKEMRNIIDSNPTLSSQDIARLEDSFDRIMEFAHDYKHGYKIITHEFALLANLSLNENLPLTLRELSTRVITSCLRNNPPVVEFINESFPNFKSKIMAALSNLNDSNHRSSNILIKRYLSILNELPVTSEDLPIYSTVVLQNVYERNNKDKQLQIKVLELISKILKADMYENDDTNLILFKRNAENWSSNLQEWANEFQEMVQNKSIDELHTRTFFDTLYNLKKIFKSDITINKGFLNWLAQQCKARQSNLDNGLQERDTEQDSFDKKLIDSRHLIFGNPMAHRIKNFRDEL

SEQ ID NO: 52 - protein, 490 aa, Kluyveromyces lactis
MRVKCVNRAIYVLTVLLFSRLVVSQVVLTPSNSNADPKQKDTANTVAAVEANNDANIAKKDAESDLVIGDHLVCNTKECYPIGFVPSTEWKEIRPGQRLPPGLDIRVSLEKGVREAKLPEPGSENIGNEEEDVKGLVLGAEGSTLSESELKETSEDLENEQSGFKLNNAEKESDILQQETDLKIAVSDNAEATSNEPAGHEFSEDFAKIKSLMQSPDEKTWEEVETLLDDLVEFAHDYKKGFKILSNEFELLEYLSFNDTLSIQIRELAARIIVSSLRNNPPSIDFVNEKYPQTTFKLCEHLSELQASQGSKLLIKRFLSILDVLLSRTEYVSIKDDVLWRLYQIEDPSSKIKILEIIAKFYNEKNEQVIDTVQQDMKTVQKWVNELTTIIQTPELDELHLRSFFHCISFIKTRFKNRVKIDSDFLNWLIDEIEVRNEKSKDDIYKRDVDQLEFDNQLAKSRHAVFGNPNAARLKERLFDDDDTLIADEL

SEQ ID NO: 53 - protein, 505 aa, Candida boidinii
MKFEFSLLVLIFSKLLVAANTAGGDMVCPDDNPDNCYPKIFVPTNEWQEIKPEQHIPAGLHVRMNIENMGREAKLPEKSSNSQINKDIQAVAVDLGGDAADNGGDVNNAVVAVGEVHDAEENIKVENGNGQGNKKSNGSRGKPAPGELLNALKGVEEFLNNDRTDNVEGLMGYLEILDDLSHDIDYGVDISKNPMSLIQLTGIYTFEQPDIYETKLKGKTTDSLKIQDMSMRVLSSTIRNNDEALDNIVELFNGSKDKLYKVIMEKLEKLNNNSFENIIQRRRLGLLNSILGHEEIASSFCCLSNDLTLLHLYSKITDKESKAKIINILHDLRIAPDYCHSENIVNLSPQDIQDSLQLKKRYQDDNLNISESVIVDEEDEEAFGDITDVDLKYSIVAQRMLRKYGLISNYKAREILQDLIDLKNNKKNSLKISSRFLNWMEYQIDQVKQLNNNLSGSNNQDDDNQQRFTIESRDGERDYLDYLIVARHEVFGNSHAGRKASADEL

SEQ ID NO: 54 - protein, 356 aa, Ogataea parapolymorpha
MLCLLLFGGVSLAKLICPDPNPLNCYPELFEPSTDWKPVKEGQIIPGGLDIRLNIDTLEREAKLTGNSQPNENGAVIVPEDIMELDEEQNLSEALRYLSKFVDHGVGDSATLLRKLEFISEMSSDSDYGVDTMQYIQPLIRLSGLYGEEGLKQIDDENRDEIRELATIILASSLRNNPEAQRKFLQYFSDPMDFVDHLTAKIQNDVLLRRRLGILGSLLNSGSLIDGFESIKKKLLILYPQLENQATKQRLMHIISDITGDVEDEDMDRQFANIAQDTLIDQKALDDGTLTLLDELKKLKLNNRNLFKAKSEFLEWLNVRMEALKAAKDPKLEEFRSLRHEIFGNPKAMRKSYDEL

SEQ ID NO: 55 - protein, 299 aa, Komagataella phaffii
MKLHLVILCLITAVYCFSAVDREIFQLNHELRQEYGDNFNFYEWLKLPKGPSSTFEDIDNAYKKLSRKLHPDKIRQKKLSQEQFEQLKKKATERYQQLSAVGSILRSESKERYDYFVKHGFPVYKGNDYTYAKFRPSVLLTIFILFALATLTHFVFIRLSAVQSRKRLSSLIEENKQLAWPQGVQDVTQVKDVKVYNEHLRKWFLVCFDGSVHYVENDKTFHVDPEEVELPSWQDTLPGKLIVKLIPQLARKPRSPKEIKKENLDDKTRKTKKPTGDSKTLPNGKTIYKATKSGGRRRK

SEQ ID NO: 56 - protein, 299 aa, Komagataella pastoris
MKLHLVILCLITAVYCFSAVDREIFQLNHELRQEFGDNFNFYEWLKLPKGPSSTFEDIDNAYKKLSRKLHPDKVRQKKLSQQQFQQLKKKATERYQQLSAVGSILRSESKERYDYFLKHGFPVYKGNDYTYAKFRPSVLITVFILFALATLTHFVFIRLSAVQSRKRLSSLIEENKQLAWPQGVQDVTKVKDVKVYNEHLRKWFLVCFDGSVHYVENDKTYHVDPEEVELPSWQDSLPGKVIVRLIPQLAKKPRPPKETKKEDLDEKSKKTKKPTGDSKTLPNGKTIYKATKSGGRRRK

SEQ ID NO: 57 - protein, 287 aa, Yarrowia lipolytica
MKFSIIFLVTLFALVFAQGGNQWSKEDREIFDLNLAVQKDLNPDNSKPVSFYQWLDTERKASVDEVTKSYRKLSRQLHPDKNRKVPGATDRFTRLGLVYKILINKDLRKRYDFYLKNGFPREGENGEFVFKRFKPGVGFALFVLYFLIGLGSYVVKYLNAKKIKSTIERVEREVRKEASRKNGVRLPATTDVIVDGRQYCYYNTGEIHLVDTDNNIEHPISSQGVEMPGIKDSLWVTLPVALFNLVKPKSAAEKAEEAKIQQEKEAKEERERPKPKAATKVGGRRRK

SEQ ID NO: 58 - protein, 414 aa, Trichoderma reesei
MKIEYLVVGVLSLLTPLAAAWSKEDREIFRIRDEIAAHESDPAASFYDILGVTPSASQDDINKAYRKKSRSLHPDKVKQQLRAEKAQADKKKGAGGGSAASSSKGPTQAEIRKAVKEASERQARLSLIANILRGPARDRYDHFLANGFPLWKGTDYYYNRYRPGLGTVLVGVFMMGGGAIHYLALYMSWKRQREFVERYVTFARNAAWGNDAGIPGVDAMPAPAPAPAPEEDEAAAPAQPRNRRERRMQEKETRKDDGKSSKKARKAVTSKSSSSAPTPTGARKRVVAENGKILVVDSQGDVFLEEEDEEGNVNEFLLDPNELLQPTFKDTAVVRVPVWVFRSTVGRFLPKGAAQAEAEETHEEDSDAAQNTPPSSESAGDDFEILDKSTDSLSKVKTSGAQQGKATKRKTTKK

SEQ ID NO: 59 - protein, 303 aa, Schizosaccharomyces pombe
MSRIFILLLLFGVCLAWTSSDLEIFRVVDSLKSILKNKATFYELLEVPTKASIKEINRAYRKKSILYHPDKNPKSKELYTLLGLIVNILRNTETRKRYDYFLKNGFPRWKGTGYLYSRYRPGLGAVLVLLFLLISIAHFVMLVISSKRQKKIMQDHIDIARQHESYATSARGSKRIVQVPGGRRIYTVDSITGQVCILDPSSNIEYLVSPDSVASVKISDTFFYRLPRFIVWNAFGRWFARAPASSEDTDSDGQMEDEEKSDSVHKSSFSSPSKKEASIKAGKRRMKRRANRIPLSKNTNREN

SEQ ID NO: 60 - protein, 295 aa, Saccharomyces cerevisiae
MNGYWKPALVVLGLVSLSYAFTTIETEIFQLQNEISTKYGPDMNFYKFLKLPKLQNSSTKEITKNLRKLSKKYHPDKNPKYRKLYERLNLATQILSNSSNRKIYDYYLQNGFPNYDFHKGGFYFSRMKPKTWFLLAFIWIVVNIGQYIISIIQYRSQRSRIENFISQCKQQDDTNGLGVKQLTFKQHEKDEGKSLVVRFSDVYVVEPDGSETLISPDTLDKPSVKNCLFWRIPASVWNMTFGKSVGSAGKEEIITDSKKYDGNQTKKGNKVKKGSAKKGQKKMELPNGKVIYSRK

SEQ ID NO: 61 - protein, 277 aa, Kluyveromyces lactis
MLSSSRPVTYALFLSLFAAVAYCFTRDEIEIFQLQQELHTKYGSNMDFYQFLKLPKLKQSTSAEITKNFKKLAKKYHPDKNPKYRKLYERINLITKLLSDEGHRKTYDYYLKNGFPKYDYKKGGFFFNRVTPSVWFTFFFLYVLAGVIHLVLLKLHNNANKKRIENFVAKVREQDTTNSLGESKLVFKESEDSEDKQLLVRFGEVFVIQPDESLAKISTDDIIDPGINDTLLVKLPKWIWNKTLGKFINIGTSKSQQPNKGSPNKNKRNSKINSKAQ

SEQ ID NO: 62 - protein, 404 aa, Candida boidinii
MRSFKIIFFVLAFFTAIALCWTHEDIEIFEINESLKKETKDPEMNFYKYLNLPSGPKSSYDQISRAFKKLSRKYHPDKYKPDFNNDEKTINKQKKNWEKRFQNIGAIAEILRSENKDRYDFFYKNGFPTINDENEYVYNKYRPSFLITLAVIFVIISVLHFIVIKSNNTQQRQRIESLINEIKTRAFGNGTPTDFKDRKVYHDGLDKYFVAKFDGSVYLLDESHLSSGTPIEELSPEEIDKIEMQRHGYNGPKLAKGVFYYKDDTYKNRRTRRSELKHGSDEDEDVLLQMSVDEVPLVTLKDMLFIRFLSSIYNTTLERLIPKSQPETETSGSKKKTIPTTKSKDSTTEEDFEILNLEDANPDSNETSKSSKEANTVLGSKTKKTSSGEKKVLPNGQVIYSRKK

SEQ ID NO: 63 - protein, 397 aa, Aspergillus niger
MKSIALRLFVFVALIVLAAAWTKEDYEIFRLNDELAAAEGPNVTFYDFLGAKPNANQDELSKAYRQKSRLLHPDKVKRSFIANSSKDKSRSKSSKSGVHVNQGPSKREIAAAVKEAHERSARLNTVANILRGPGRERYDHFLKNGFPKWKGTGYYYSRFRPGLGSVLIGLFLVFGGGAHYAALVLGWKRQREFVDRYIRQARRAAWGDESGVRGIPGLDGASAPAPTPAPAPEPEQSAMPMNRRQKRMMDRENRKEGKKGGRAASRNSGTATPTSEPQMEPSGERKKVIAENGKVLIVDSLGNVFLEEETEDGERQEFLLDVDEIQRPTIRDTLVFRLPGWVYSKTVGRLLGSSNAVNSGAESEEEPSEIVEESTEGAASSARSSKARRRGKRSQRS

SEQ ID NO: 64 - protein, 323 aa, Ogataea parapolymorpha
MRLLFWLAIFSATVFAAWSAEDLEIFKLQHELVKDTKKETNFYEYLGLSNGPKASYDEINKAYKKMSRKLHPDKVRRKEGMSQKAFERRKKAAEQRFQRLSLIGTILRGERKERYDYYLKHGFPAYTGTGFALSKFRPGPVLALVVVVVLFSAVHYIMLKLNTQQKRKRVESLINDLKAKAFGPSMLPGTNFSDQRVAHMDKLFVVKFDGSVWLVDKELKEGEDYIVDEDGRQIFRVEAEPKNRKQRRAKKDKDEVLLPVTPDDVEEVTWRDTLVVRFVLWAISKLEKKPKTHDKADKGTIRRLPNGKVKKVRPTGENGEKNK

SEQ ID NO: 65 - protein, 53 aa, Komagataella phaffii
RRVERILRNRRAAHASREKKRRHVEFLENHVVDLESALQESAKATNKLKEIQD

SEQ ID NO: 66 - protein, 53 aa, Komagataella pastoris
RRVERILRNRRAAHASREKKRRHVEFLENHVVDLESALQESAKATNKLKQIQD

SEQ ID NO: 67 - protein, 45 aa, Yarrowia lipolytica
RRIERIMRNRQAAHASREKKRRHLEDLEKKCSELSSENNDLHHQV

SEQ ID NO: 68 - protein, 53 aa, Trichoderma reesei
RRVERVLRNRRAAQSSRERKRLEVEALEKRNKELETLLINVQKTNLILVEELN

SEQ ID NO: 69 - protein, 51 aa, Saccharomyces cerevisiae
RRIERILRNRRAAHQSREKKRLHLQYLERKCSLLENLLNSVNLEKLADHED

SEQ ID NO: 70 - protein, 46 aa, Kluyveromyces lactis
RRIERILRNRRAAHQSREKKRLHVQRLEEKCHLLEGILKMVDLDIL

SEQ ID NO: 71 - protein, 48 aa, Candida boidinii
RRVERILRNRRAAHASREKKRKHVEYLELYVNNLENGIKNYISNQEKL

SEQ ID NO: 72 - protein, 53 aa, Aspergillus niger
RRIERVLRNRAAAQTSRERKRLEMEKLENEKIQMEQQNQFLLQRLSQMEAENN

SEQ ID NO: 73 - protein, 39 aa, Ogataea angusta
RRVERILRNRRAAHASREKKRRHVEYLENYVTDLESALA

SEQ ID NO: 74 - protein, 331 aa, Komagataella phaffii
MPVDSSHKTASPLPPRKRAKTEEEKEQRRVERILRNRRAAHASREKKRRHVEFLENHVVDLESALQESAKATNKLKEIQDIIVSRLEALGGTVSDLDLTVPEVDFPKSSDLEPMSDLSTSSKSEKASTSTRRSLTEDLDEDDVAEYDDEEEDEELPRKMKVLNDKNKSTSIKQEKLNELPSPLSSDFSDVDEEKSTLTHLKLQQQQQQPVDNYVSTPLSLPEDSVDFINPGNLKIESDENFLLSSNTLQIKHENDTDYITTAPSGSINDFFNSYDISESNRLHHPAVMTDSSLHITAGSIGFFSLIGGGESSVAGRRSSVGTYQLTCIAIR

SEQ ID NO: 75 - protein, 330 aa, Komagataella pastoris
MPVDSSHKIASPLPPRKRAKTEEEKEQRRVERILRNRRAAHASREKKRRHVEFLENHVVDLESALQESAKATNKLKQIQDIIVSRLEALGGTVSDLDLAVPEVDFPKFSDLELSTDLSSSTKSEKASTSTCRSSTEDLDEDGVAEYDDEEDEELPRKKNVLNDKSKNRTIKQEKLNELPSPLSSDFSDVDEEKSTLTHFQLQQQQQQQPVDNYVSTPLSLPEDSIDFINPGSLKIESDENFLLGSSTLQIKHENDTEYIPTAPSGSINDFFNSYDISESNRLHHPAVMTDSSLHTTAGSIGFFSLIRGKSFVVGRRSSVGVYQLTCIAIR

SEQ ID NO: 76 - protein, 299 aa, Yarrowia lipolytica
MSIKREESFTPTPEDLGSPLTADSPGSPESGDKRKKDLTLPLPAGALPPRKRAKTENEKEQRRIERIMRNRQAAHASREKKRRHLEDLEKKCSELSSENNDLHHQVTESKKTNMHLMEQHYSLVAKLQQLSSLVNMAKSSGALAGVDVPDMSDVSMAPKLEMPTAAPSQPMGLASAPTLFNHDNETVVPDSPIVKTEEVDSTNFLLHTESSSPPELAESTGSGSPSSTLSCDETDYLVDRARHPAVMTVATTDQQRRHKISFSSRTSPLTTSLDCMDCRMTSPCLKTTSSLPSTTLLLI

SEQ ID NO: 77 - protein, 451 aa, Trichoderma reesei
MAFQQSSPLVKFEASPAESFLSAPGDNFTSLFADSTPSTLNPRDMMTPDSVADIDSRLSVIPESQDAEDDESHSTSATAPSTSEKKPVKKRKSWGQVLPEPKTNLPPRKRAKTEDEKEQRRVERVLRNRRAAQSSRERKRLEVEALEKRNKELETLLINVQKTNLILVEELNRFRRSSGVVTRSSSPLDSLQDSITLSQQLFGSRDGQTMSNPEQSLMDQIMRSAANPTVNPASLSPSLPPISDKEFQTKEEDEEQADEDEEMEQTWHETKEAAAAKEKNSKQSRVSTDSTQRPAVSIGGDAAVPVFSDDAGANCLGLDPVHQDDGPFSIGHSFGLSAALDADRYLLESQLLASPNASTVDDDYLAGDSAACFTNPLPSDYDFDINDFLTDDANHAAYDIVAASNYAAADRELDLEIHDPENQIPSRHSIQQPQSGASSHGCDDGGIAVGV

SEQ ID NO: 78 - protein, 238 aa, Saccharomyces cerevisiae
MEMTDFELTSNSQSNLAIPTNFKSTLPPRKRAKTKEEKEQRRIERILRNRRAAHQSREKKRLHLQYLERKCSLLENLLNSVNLEKLADHEDALTCSHDAFVASLDEYRDFQSTRGASLDTRASSHSSSDTFTPSPLNCTMEPATLSPKSMRDSASDQETSWELQMFKTENVPESTTLPAVDNNNLFDAVASPLADPLCDDIAGNSLPFDNSIDLDNWRNPEAQSGLNSFELNDFFITS

SEQ ID NO: 79 - protein, 273 aa, Kluyveromyces lactis
MTGKNSVSDIPVNFKPTLPPRKRAKTQEEKEQRRIERILRNRRAAHQSREKKRLHVQRLEEKCHLLEGILKMVDLDILSENNAKLSGMVEQWREMQVSDSGSISSHDSNTGMLDSPESLTSSPDKKDHYSHSSHSTSISSSSSSSSPSNLPHGMVTDNGMLDEDNNSLNYILGQQNYQLSSTPVVKLEEDHSMLLENNGDADLNDVGISFIAEDGTNSDNKNIDMRNQETGEGWNLLLTVPPELNSDLSELEPSDIISPIGLDTWRNPAVIVT

SEQ ID NO: 80 - protein, 351 aa, Candida boidinii
MSLSNTPSSPDNISNVSASLISSNLKGKTDELLKSASAIGLLPPRKRAKTAEEKEQRRVERILRNRRAAHASREKKRKHVEYLELYVNNLENGIKNYISNQEKLINFQSLLIAKLKVANVDISDIDLSTCTNIDIVSIEKPECLNYSPNSSSKKNKKSSSDDEEEEDDDDDDEDDEDDNVELKHKSNSQKQQQQQQKEYKEVEQSTKQDESKTSNQQQEQEQEQEQVSTPKAELTQQLSDPTMDMKFKSAVKLEDVNQLPQDQYLMSPPNTESPRKFILDSSNINKDYTHIFVGDDLLFNNDLQLCSDSLKQQELNVPNIENIISDYSLDSMNDLNAYNRLHHPAAMVQRY

SEQ ID NO: 81 - protein, 342 aa, Aspergillus niger
MMEEAFSPVDSLAGSPTPELPLLTVSPADTSLDDSSVQAGETKAEEKKPVKKRKSWGQELPVPKTNLPPRKRAKTEDEKEQRRIERVLRNRAAAQTSRERKRLEMEKLENEKIQMEQQNQFLLQRLSQMEAENNRLNQQVAQLSAEVRGSRGNTPKPGSPVSASPTLTPTLFKQERDEIPLERIPFPTPSITDYSPTLRPSTLAESSDVTQHPAVSVAGLEGEGSALSLFDVGSNPEPHAADDLAAPLSDDDFHRLFNVDSPVGSDSSVLEDGFAFDVLDGGDLSAFPFDSMVDFDPESVGFEGIEPPHGLPDETSRQTSSVQPSLGASTSRCDGQGIAAGC

SEQ ID NO: 82 - protein, 325 aa, Ogataea angusta
MTALNSSVQHQEVSSDLPFGTLPPRKRAKTEEEKEQRRVERILRNRRAAHASREKKRRHVEYLENYVTDLESALATHEGNYRKMAKIQSSLISLLSEHGIDYSSVDLAVEPCPKVERPEGLELTGSIPVKKQKIASAKSPKSLSRKSKSEIPSPSFDENIFSEEENEHDDGIEEYGKAGQEATEAPSLSHNRKRKAQDAYISPPGSTSPSKLKLEEDERISKHEYSNLFDDTDDIFPSEKSSSLELYKQDDLTMASFVKQEEEEMVPFVKQEDEFKFPDSGFNADDCHLIQVEDLCSFNSVHHPAAAPLTAESIDNHFEFDDYLS

SEQ ID NO: 83 - protein, 223 aa, Pichia pastoris
MSTTKPMQVLAPDLTETPKTYSLGVHLGKGKDKLQDPTELYSMILDGMDHSQLNSFINDQLNLGSLRLPANPPAASGAKRGANVSSINMDDLQTFDFNFDYERDSSPLELNMDSQSLMFSSPEKAPCGSLPSQHQPHSQVAAAQGTTINPRQLSTSSASSFVSSDFDVDSLLADEYAEKLEYGAISSASSSICSNSVLPSQGVTSQHSSPIEQRPRVGNSKRL

SEQ ID NO: 84 - protein, 42 aa, Artificial sequence
  synthetic transcription activator domain (VP64)
GGGGSDALDDFDLDMLGSDALDDFDLDMLGSDALDDFDLDML

SEQ ID NO: 85 - protein, 14 aa, Pichia pastoris
EPRKKETKQRKRAK

SEQ ID NO: 86 - protein, 7 aa, Artificial sequence
  nuclear localization signal of synMSN4
PKKKRKV

SEQ ID NO: 87 - protein, 54 aa, Artificial sequence
  Consensus sequence
  MISC_FEATURE (10)..(10): K at position 10 can be interchangeable with R
  MISC_FEATURE (11)..(11): R at position 11 can be interchangeable with K
  MISC_FEATURE (15)..(15): Xaa can be Q or S
  MISC_FEATURE (19)..(19): K at position 19 can be interchangeable with R
  misc_feature (22)..(22): Xaa can be any naturally occurring amino acid
  MISC_FEATURE (25)..(25): Xaa can be V or L
  MISC_FEATURE (27)..(27): S at position 27 can be interchangeable with T
  misc_feature (28)..(28): Xaa can be any naturally occurring amino acid
  MISC_FEATURE (30)..(30): K at position 30 can be interchangeable with R
  misc_feature (33)..(33): Xaa can be any naturally occurring amino acid
  misc_feature (35)..(36): Xaa can be any naturally occurring amino acid
  misc_feature (38)..(38): Xaa can be any naturally occurring amino acid
  MISC_FEATURE (40)..(40): K at position 40 can be interchangeable with R
  MISC_FEATURE (44)..(44): S at position 44 can be interchangeable with T
  misc_feature (48)..(48): Xaa can be any naturally occurring amino acid
  MISC_FEATURE (52)..(52): R at position 52 can be interchangeable with K
KPFVCTLCSKRFRRXEHLKRHXRSXHSXEKPFXCXXCXKKFSRSDNLXQHLRTH

SEQ ID NO: 88 - DNA, 1098 bp, Pichia pastoris
gataggtctctcatgtctacaacaaaaccaatgcaggtgttagccccggaccttactgagacaccaaagacatattcgttaggtgtccatttggggaaaggcaaggacaaactccaggatccgacagaactctactcgatgatcctagatggaatggatcactcacagctcaattcttttattaacgatcagttgaacttgggatcattgcgcttgccggcgaatcctcctgctgcaagtggtgctaaacggggtgcaaatgtcagttctatcaacatggatgatttacaaacgtttgatttcaactttgattacgaacgggattcatcgccgctagaattgaacatggattctcaatctttgatgttttcctctccagagaaagctccctgtggctccttgccgtctcagcatcagcctcactctcaggtcgcagccgcacagggaactaccatcaatccaaggcagttatccacatcttctgccagtagctttgtatcttcggattttgatgttgattcactcctggcagacgagtacgctgagaaactagaatatggagccatatcatctgcctcatcttccatctgttcgaattctgttcttcctagccagggcgtaacttcgcaacatagctctcctatagaacaaagacctcgtgtgggaaattccaaacgcttgagtgatttttggatgcaggacgaagctgtcactgccatttccacctggctcaaagctgaaataccttcctccttggctacgccggctcctacagtcacacaaataagtagtcccagccttagcaccccagagccaaggaagaaagaaacaaaacaaagaaagagggcaaagtccatagacacgaatgagcgatctgaacaagtagcagcttctaattcagatgatgaaaagcaattccgctgcacggattgcagtagacgcttccgcagatcagaacacctgaaacgacatcataggtctgttcattctaacgaaaggccgttccattgtgctcactgtgataaacggttctcaagaagcgacaacttgtcgcagcatctacgtactcaccgtaagcagtgagcttagagacctatc

SEQ ID NO: 89 - DNA, 36 bp, Artificial sequence
  oligonucleotide primer PP7435_Chr2-0555
gataggtctctcatgtctacaacaaaaccaatgcag

SEQ ID NO: 90 - DNA, 35 bp, Artificial sequence
  oligonucleotide primer PP7435_Chr2-0555 reverse
gataggtctctaagctcactgcttacggtgagtac

SEQ ID NO: 91 - DNA, 469 bp, Artificial sequence
  synMSN4
gatctaggtctcacatgggtaagccaattcctaacccattgttgggtttggattctactccaaaaaagaagagaaaggttggtggaggtggatctgatgcccttgacgattttgacttggacatgttgggttctgacgctttggatgactttgatcttgatatgcttggttccgacgctctagatgatttcgacttggatatgctgggatccgatgccttggacgatttcgacttggatatgttgggtggaggtggatctaattcagatgatgaaaagcaattccgctgcacggattgcagtagacgcttccgcagatcagaacacctgaaacgacatcataggtctgttcattctaacgaaaggccgttccattgtgctcactgtgataaacggttctcaagaagcgacaacttgtcgcagcatctacgtactcaccgtaagcagtgataggcttcgagaccaatgac

SEQ ID NO: 92 - DNA, 36 bp, Artificial sequence
  oligonucleotide primer syMSN4
gatctaggtctcacatgggtaagccaattcctaacc

SEQ ID NO: 93 - DNA, 37 bp, Artificial sequence
  oligonucleotide primer synMSN4 reverse
gtcattggtctcgaagcctatcactgcttacggtgag

SEQ ID NO: 94 - DNA, 2142 bp, Saccharomyces cerevisiae
gataggtctcgcatgacggtcgaccatgatttcaatagcgaagatattttattccccatagaaagcatgagtagtatacaatacgtggagaataataacccaaataatattaacaacgatgttatcccgtattctctagatatcaaaaacactgtcttagatagtgcggatctcaatgacattcaaaatcaagaaacttcactgaatttggggcttcctccactatctttcgactctccactgcccgtaacggaaacgataccatccactaccgataacagcttgcatttgaaagctgatagcaacaaaaatcgcgatgcaagaactattgaaaatgatagtgaaattaagagtactaataatgctagtggctctggggcaaatcaatacacaactcttacttcaccttatcctatgaacgacattttgtacaacatgaacaatccgttacaatcaccgtcaccttcatcggtacctcaaaatccgactataaatcctcccataaatacagcaagtaacgaaactaatttatcgcctcaaacttcaaatggtaatgaaactcttatatctcctcgagcccaacaacatacgtccattaaagataatcgtctgtccttacctaatggtgctaattcgaatcttttcattgacactaacccaaacaatttgaacgaaaaactaagaaatcaattgaactcagatacaaattcatattctaactccatttctaattcaaactccaattctacgggtaatttaaattccagttattttaattcactgaacatagactccatgctagatgattacgtttctagtgatctcttattgaatgatgatgatgatgacactaatttatcacgccgaagatttagcgacgttataacaaaccaatttccgtcaatgacaaattcgaggaattctatttctcactctttggacctttggaaccatccgaaaattaatccaagcaatagaaatacaaatctcaatatcactactaattctacctcaagttccaatgcaagtccgaataccactactatgaacgcaaatgcagactcaaatattgctggcaacccgaaaaacaatgacgctaccatagacaatgagttgacacagattcttaacgaatataatatgaacttcaacgataatttgggcacatccacttctggcaagaacaaatctgcttgcccaagttcttttgatgccaatgctatgacaaagataaatccaagtcagcaattacagcaacagctaaaccgagttcaacacaagcagctcacctcgtcacataataacagtagcactaacatgaaatccttcaacagcgatctttattcaagaaggcaaagagcttctttacccataatcgatgattcactaagctacgacctggttaataagcaggatgaagatcccaagaacgatatgctgccgaattcaaatttgagttcatctcaacaatttatcaaaccgtctatgattctttcagacaatgcgtccgttattgcgaaagtggcgactacaggcttgagtaatgatatgccatttttgacagaggaaggtgaacaaaatgctaattctactccaaatttcgatctttccatcactcaaatgaatatggctccattatcgcctgcatcatcatcctccacgtctcttgcaacaaatcatttctatcaccatttcccacagcagggtcaccataccatgaactctaaaatcggttcttcccttcggaggcggaagtctgctgtgcctttgatgggtacggtgccgcttacaaatcaacaaaataatataagcagtagtagtgtcaactcaactggcaatggtgctggggttacgaaggaaagaaggccaagttacaggagaaaatcaatgacaccgtccagaagatcaagtgtcgtaatagaatcaacaaaggaactcgaggagaaaccgttccactgtcacatttgtcccaagagctttaagcgcagcgaacatttgaaaaggcatgtgagatctgttcactctaacgaacgaccatttgcttgtcacatatgcgataagaaatttagtagaagcgataatttgtcgcaacacatcaagactcataaaaaacatggagacatttaagcttggagacctatc

SEQ ID NO: 95 - DNA, 28 bp, Artificial sequence
  oligonucleotide primer YMR037C
gataggtctcgcatgacggtcgaccatg

SEQ ID NO: 96 - DNA, 42 bp, Artificial sequence
  oligonucleotide primer YMR037C reverse
gataggtctccaagcttaaatgtctccatgttttttatgagt

SEQ ID NO: 97 - DNA, 1920 bp, Saccharomyces cerevisiae
gactggtctcacatgctagtctttggacctaatagtagtttcgttcgtcacgcaaacaagaaacaagaagattcgtctataatgaacgagccaaacggattgatggacccggtattgagcacaaccaacgtttctgctacttcttctaatgacaattctgcgaacaatagcatatcttcgccggaatatacctttggtcaattctcaatggattctccgcatagaacggacgccactaatactccaattttaacagcgacaactaatacgactgctaataatagtttaatgaatttaaaggataccgccagtttagctaccaactggaagtggaaaaattccaataacgcacagttcgtgaatgacggtgagaaacaaagcagtaatgctaatggtaagaaaaatggtggtgataagatatatagttcagtagccacccctcaagctttaaatgacgaattgaaaaacttggagcaactagaaaaggtattttctccaatgaatcctatcaatgacagtcattttaatgaaaatatagaattatcgccacaccaacatgcaacttctcccaagacaaaccttcttgaggcagaaccttcaatatattccaatttgtttctagatgctaggttaccaaacaacgccaacagtacaacaggattgaacgacaatgattataatctagacgataccaataatgataatactaatagcatgcaatcaatcttagaggattttgtatcttcagaagaagcattgaagttcatgccggacgctggtcgcgacgcaagaagatacagcgaggtggttacctcttcctttccttctatgacggattctagaaattcgatctctcattcgatagagttttggaatctcaatcacaaaaatagtagcaacagtaaacccactcaacaaattatccctgaaggtactgccactactgagaggcgtggatcaaccatttcacctactaccactataaacaactctaatccaaacttcaaattattagatcatgacgtttctcaagctctgagcggttatagtatggatttttctaaggactctggtataacaaagccaaaaagcatttcctcttctttaaatcgcatctcccatagcagtagcaccacaaggcaacagcgtgcctctttgcccttaattcatgatattgaatcttttgcaaatgattcggtgatggcaaatcctctgtctgattccgcatcatttctttcagaagaaaatgaagatgatgcttttggtgcgctaaattacaatagcttagatgcaaccacaatgtcggcattcgacaataacgtagaccccttcaacattctcaagtcatctccggctcaggatcaacagtttatcaaaccctctatgatgttgtcggataatgcctctgctgccgctaaattggcgacttctggtgttgataatatcacacctacaccagctttccaaagaagaagctatgatatctcgatgaactcttcgttcaaaatacttcctactagtcaagctcaccatgcagctcaacatcatcaacaacaacctactaaacaggcaacggtaagcccaaacacaagaagaagaaagtcgtcaagtgttactttaagtccaactatttctcataacaacaacaatggtaaggttcctgtccaacctcggaaaaggaaatctattactaccattgaccccaacaactacgataaaaataaacctttcaagtgtaaagactgtgagaaggcattcagacgcagtgagcacttgaaaaggcatataagatccgttcattcaacggaacgcccttttgcttgtatgttctgtgagaaaaaattcagtagaagtgacaatttatcacaacatctaaaaactcacaaaaagcacggtgatttttgagcttggagacctatc

SEQ ID NO: 98 - DNA, 38 bp, Artificial sequence
  oligonucleotide primer YKL062W
gactggtctcacatgctagtctttggacctaatagtag

SEQ ID NO: 99 - DNA, 33 bp, Artificial sequence
  oligonucleotide primer YKL062W reverse
gataggtctccaagctcaaaaatcaccgtgctt

SEQ ID NO: 100 - DNA, 885 bp, Yarrowia lipolytica
gataggtctcacatggacctcgaattggaaattcccgtcttgcattccatggactcgcaccaccaggtggtggactcccacagactggcacagcaacagttccagtaccagcagatccacatgctgcagcagacgctgtcacagcagtacccccacaccccatccaccacaccccccatttacatgctgtcgcctgcggactacgagaaggacgccgtttccatctcaccggtaatgctgtggcccccctcggcccactcccaggcctcttaccattacgagatgccctccgttatctcgccatctccttctcccactagatccttctgtaatccgagagagctggaggttcaggacgagctcgagcagcttgaacagcagcccgccgctctctccgtcgaacatctgtttgacattgagaactcatcgatcgagtatgcacacgacgagctgcatgacacctcttcgtgctccgactcgcagtcgagcttttcccctcagcagtcccctgcctccccggcctccacttactcgcctctcgaggacgagtttctcaacttggctggatccgagttgaagagcgagcccagcgcggacgacgagaaggatgatgtggacacggagcttccccagcagcccgagatcatcatccctgtgtcgtgccgaggccgaaagccgtccatcgacgactccaaaaagacttttgtctgcacccactgccagcgtcggttccggcgccaggagcatctcaagcgacatttccgatccctacacactcgagagaagcctttcaactgcgacacgtgcggcaagaagttttctcggtcggacaatctcgcccagcatatgcgtacgcatcctcgggactaggctttgagaccagtc

SEQ ID NO: 101 - DNA, 29 bp, Artificial sequence
  oligonucleotide primer YALI0B21582
gataggtctcacatggacctcgaattgga

SEQ ID NO: 102 - DNA, 31 bp, Artificial sequence
  oligonucleotide primer YALI0B21582 reverse
gactggtctcaaagcctagtcccgaggatgc

SEQ ID NO: 103 - DNA, 2001 bp, Aspergillus niger
gataggtctcacatggacggaacatacaccatggcacctacttcggtgcaaggtcaaccatcatttgcatactacgctgattcgcagcaaagacaacatttcaccagccacccctcagatatgcagtcatactatggccaagtgcaggccttccagcaacaaccacagcactgcatgccggagcagcagacactctacactgcccctctcatgaacatgcaccagatggctaccaccaatgccttccgtggtgccatgaacatgactcccattgcctctcctcagccgtcacacctcaagcccacaattgttgtgcagcagggctctcccgccctgatgcctctggacacgaggttcgtcggtaacgactactacgcattcccctccaccccaccactctccacagctggaagctctatcagcagcccgccttctaccagcggcacccttcacaccccgatcaatgacagcttcttcgctttcgagaaggtggaaggtgtcaaggagggatgcgagggagacgtccatgcagagattctggccaatgctgactgggcccggtctgactcgccgcctcttacacctggtaagtcattatctaacccgatgtcccttttttacatggttgcaagataggctgcagggagtgggtgcagccaacggaaaaggcacggggccgggcatctagggttgtacagggagactaactcgacttgttctagtgttcatccatccgccttccctcaccgccagccaaacatccgagcttctgtcagcgcacagctcttgcccatccctttccccatcgccatctcccgtggtccccacattcgttgcccagcctcaaggtctgccgaccgagcagtccagctccgacttctgtgacccccgtcagctgacggttgagtcctccatcaatgccacccctgctgagctgccgcctctgcccacgctctcctgcgatgacgaggagcctcgggtggttctgggcagcgaggccgtgacccttcctgtccatgaaaccctctctcccgccttcacctgctcctcttcggaggaccctctcagcagcctgccgacctttgacagcttctcggacctggactcggaagatgaattcgtcaaccgcctggtcgacttcccccctagtggcaatgcctactacttgggtgagaagaggcagcgcgtgggaacgacatacccccttgaggaagaggaattcttcagtgagcagagcttcgacgagtctgacgagcaagatctctctcagtccagtctcccttacctgggaagccacgacttcactggcgtccagacgaacatcaatgaagcttcggaagagatgggcaacaagaagaggaacaaccgcaagtcgctgaagcgggctagtacctcggacagcgaaacggattcgattagcaagaagtcgcagccttcgatcaacagccgtgccaccagcactgagacaaacgcctcgacaccccagactgtccaggcccgccacaactccgatgcgcattcgtcgtgcgcttctgaggctcctgctgcccccgtctcggtcaaccgacgcggtcgtaagcagtccctgacggatgacccctccaagaccttcgtgtgcaccctctgctcccgtcgcttccgtcgccaagagcacctcaagcgtcactaccgctctctccacactcaggacaagcctttcgagtgcaatgagtgcggtaagaagttctcgcggagcgataaccttgcgcagcacgctcgcactcatgcgggtggctctgtcgtgatgggcgtcatcgacaccggcaatgcgaccccgccaaccccctatgaagaacgagatcccagtacgctgggaaatgttctctacgaggccgccaacgccgccgctaccaagtccacaaccagtgagtcggatgagagttcctctgactcgccggttgccgaccgacgggcgcccaagaagcgcaagcgcgacagcgatgcctaggcttggagaccatc

SEQ ID NO: 104 - DNA, 30 bp, Artificial sequence
  oligonucleotide primer An04g03980
gataggtctcacatggacggaacatacacc

SEQ ID NO: 105 - DNA, 29 bp, Artificial sequence
  oligonucleotide primer An04g03980 reverse
gatggtctccaagcctaggcatcgctgtc

SEQ ID NO: 106 - DNA, 2068 bp, Pichia pastoris
gatctaggtctcccatgctgtcgttaaaaccatcttggctgactttggcggcattaatgtatgccatgctattggtcgtagtgccatttgctaaacctgttagagctgacgatgtcgaatcttatggaacagtgattggtatcgatttgggtaccacgtactcttgtgtcggtgtgatgaagtcgggtcgtgtagaaattcttgctaatgaccaaggtaacagaatcactccttcctacgttagtttcactgaagatgagagactggttggtgatgctgctaagaacttagctgcttctaacccaaaaaacaccatctttgatattaagagattgatcggtatgaagtatgatgccccagaggtccaaagagacttgaagcgtctgccttacactgtcaagagcaagaacggccaacctgtcgtttctgtcgagtacaagggtgaggagaagtctttcactcctgaggagatttccgccatggtcttgggtaagatgaagttgatcgctgaggactacttaggaaagaaagtcactcatgctgtcgttaccgttccagcctacttcaacgacgctcaacgtcaagccactaaggatgccggtctaatcgccggtttgactgttctgagaattgtgaacgagcctaccgccgctgcccttgcttacggtttggacaagactggtgaggaaagacagatcatcgtctacgacttgggtggaggaaccttcgatgtttctctgctttctattgagggtggtgctttcgaggttcttgctaccgccggtgacacccacttgggtggtgaggactttgactacagagttgttcgccacttcgttaagattttcaagaagaagcataacattgacatcagcaacaatgataaggctttaggtaagctgaagagagaggtcgaaaaggccaagcgtactttgtcctcccagatgactaccagaattgagattgactctttcgtcgacggtatcgacttctctgagcaactgtctagagctaagtttgaggagatcaacattgaattattcaagaaaacactgaaaccagttgaacaagtcctcaaagacgctggtgtcaagaaatctgaaattgatgacattgtcttggttggtggttctaccagaattccaaaggttcaacaattattggaggattactttgacggaaagaaggcttctaagggaattaacccagatgaagctgtcgcatacggtgctgctgttcaggctggtgttttgtctggtgaggaaggtgtcgatgacatcgtcttgcttgatgtgaaccccctaactctgggtatcgagactactggtggcgttatgactaccttaatcaacagaaacactgctatcccaactaagaaatctcaaattttctccactgctgctgacaaccagccaactgtgttgattcaagtttatgagggtgagagagccttggctaaggacaacaacttgcttggtaaattcgagctgactggtattccaccagctccaagaggtactcctcaagttgaggttacttttgttttagacgctaacggaattttgaaggtgtctgccaccgataagggaactggaaaatccgagtccatcaccatcaacaatgatcgtggtagattgtccaaggaggaggttgaccgtatggttgaagaggccgagaagtacgccgctgaggatgctgcactaagagaaaagattgaggctagaaacgctctggagaactacgctcattcccttaggaaccaagttactgatgactctgaaaccgggcttggttctaaattggacgaggacgacaaagagacattgacagatgccatcaaagataccctagagttcttggaagataacttcgacaccgcaaccaaggaagaattagacgaacaaagagaaaagctttccaagattgcttacccaatcacttctaagctatacggtgctccagagggtggtactccacctggtggtcaaggttttgacgatgatgatggagactttgactacgactatgactatgatcatgatgagttgtaagcttggagaccaatgac

SEQ ID NO: 107 - DNA, 35 bp, Artificial sequence
  oligonucleotide primer PP7435_Chr2-1167
gatctaggtctcccatgctgtcgttaaaaccatct

SEQ ID NO: 108 - DNA, 45 bp, Artificial sequence
  oligonucleotide primer PP7435_Chr2-1167 reverse
gtcattggtctccaagcttacaactcatcatgatcatagtcatag

SEQ ID NO: 109 - DNA, 949 bp, Pichia pastoris
gatctaggtctcacatgcccgtagattcttctcataagacagctagcccacttccacctcgtaaaagagcaaagacggaagaagaaaaggagcagcgtcgagtggaacgtatcctacgtaataggagagcggcccatgcttccagagagaagaaacgtagacacgttgaatttctggaaaaccacgtcgtcgacctggaatctgcacttcaagaatcagccaaagccactaacaagttgaaagaaatacaagatatcattgtttcaaggttggaagccttaggtggtaccgtctcagatttggatttaacagttccggaagtcgattttcccaaatcttctgatttggaacccatgtctgatctctcaacttcttcgaaatcggagaaagcatctacatccactcgcagatctttgactgaggatctggacgaagatgacgtcgctgaatatgacgacgaagaagaggacgaagagttacccaggaaaatgaaagtcttaaacgacaaaaacaagagcacatctatcaagcaggagaagttgaatgaacttccatctcctttgtcatccgatttttcagacgtagatgaagaaaagtcaactctcacacatttaaagttgcaacagcaacaacaacaaccagtagacaattatgtttctactcctttgagtctgccggaggattcagttgattttattaacccaggtaacttaaaaatagagtccgatgagaacttcttgttgagttcaaatactttacaaataaaacacgaaaatgacaccgactacattactacagctccatcaggttccatcaatgatttttttaattcttatgacattagcgagtcgaatcggttgcatcatccagcagcaccatttaccgctaatgcatttgatttaaatgactttgtattcttccaggaatagtaggcttcgagaccaatgac

SEQ ID NO: 110 - DNA, 33 bp, Artificial sequence
  oligonucleotide primer PP7435_Chr1-0700
gatctaggtctcacatgcccgtagattcttctc

SEQ ID NO: 111 - DNA, 42 bp, Artificial sequence
  oligonucleotide primer PP7435_Chr1-0700 reverse
gtcattggtctcgaagcctactattcctggaagaatacaaag

SEQ ID NO: 112 - DNA, 918 bp, Artificial sequence
  codon-optimized HAC1
atgccagttgatagttcgcacaagactgcttctccactgccacctagaaagagagctaagactgaggaggaaaaggagcaacgtagagtcgagagaatcctgagaaaccgtagagccgctcacgcctctagagagaaaaagagaaggcatgttgaatttcttgaaaaccacgtcgtcgatctcgaatctgcccttcaagagtcagctaaagctaccaacaagctaaaggaaattcaagacattatcgtatctagactggaggcacttggtggtactgtttctgacctggatcttacagttccagaagttgacttcccaaaatccagtgatctagaacctatgtctgatctatctacctcaagcaagtctgagaaggcaagcacgtcaaccagacgttccctaactgaggacctggacgaagatgatgtcgctgaatacgatgacgaggaggaggatgaggaactgcctagaaaaatgaaggttcttaacgacaaaaacaagtctacctctatcaaacaggaaaagctcaacgaactcccatcccctctctcttccgacttctccgacgtggacgaggaaaagtctactttgacccacctgaagttgcaacaacaacagcaacaacctgttgacaactatgtctccactcctctctcactcccagaggactcggttgacttcatcaaccccggtaaccttaagattgaatctgacgagaacttccttctatcctctaataccttacagattaagcatgaaaatgatactgactacattactaccgctccatccggatctatcaatgacttcttcaattcttacgacatttctgagtccaacagattgcaccacccagctgcaccttttacagccaacgcttttgacctaaacgacttcgtgtttttccaggagtaatag

SEQ ID NO: 113 - DNA, 2716 bp, Pichia pastoris
gatctaggtctcccatgagaacacaaaagatagtaacagtactttgtttgctactaaatactgtgcttggagctctgttgggcatcgattatggtcaagagtttactaaggctgtcctagtggctcctggtgtcccttttgaagttatcttgactccagactccaaacgtaaagataattcaatgatggccatcaaggaaaattccaaaggtgaaattgagagatattatggatcctcagctagttctgtttgtatcagaaaccctgaaacttgcttgaatcatctgaagtcattgataggtgtttcaattgatgacgtttcaactatagattacaagaagtaccattcaggtgctgagatggttccatccaaaaataacaggaacacggttgcctttaagttgggctcttctgtatatcctgtagaagagatacttgctatgagtttagatgacattaaatctagagctgaagatcatttaaaacacgcggtgccaggttcctattcagttatcagtgatgctgtcatcacagtacccactttttttacccaatcgcaaagactggccttgaaagatgctgccgaaattagtggcttaaaagtcgttggcttggttgatgacggtatatctgtggccgttaactatgcctcttcaaggcagttcaatggagacaaacaatatcatatgatctatgacatgggggctggttctttacaggcgactttggtttctatatcttccagtgatgatggtggaattgttattgatgtagaggctattgcctatgacaagtcgctgggaggccagttgttcacacaatctgtttatgacatccttttgcagaagttcttgtctgagcatccttcctttagcgagtccgacttcaacaagaatagtaaatctatgtcaaaactttggcaagcggctgaaaaggcaaagacaattttgagtgcaaacactgacacaagagtttccgttgaatccttatacaatgacattgactttagagccacaatagcaagagacgaattcgaagattacaatgcagagcatgttcataggatcactgctcctatcatcgaggccttaagtcatccattgaatgggaatctgacgtcaccttttccactgaccagtttaagttcagtaattctcacaggcgggtcaacaagagtgccgatggtgaaaaagcacctagaatctttgctaggatctgaattgattgcaaagaatgttaacgctgatgagtcagccgtttttggttctactctccgtggtgtaactttatcgcaaatgttcaaagcgaaacagatgaccgtaaatgaaagaagtgtatatgactattgcctaaaagttggttcttcagagataaacgtgttcccagttggcacccctcttgctactaagaaagtggtcgagctggaaaatgtagacagtgagaaccagctcacgattgggctctacgagaacggacaattgtttgccagtcatgaggttacagacctcaagaagagtatcaaatctctaactcaagaaggtaaagagtgttctaatattaattacgaggctacagtcgagttatctgagagcagattgctttctttaactcgtctgcaggccaaatgtgctgacgaggctgaatatttacctcctgtggacacagagtctgaggatactaaatctgaaaactcaactactagtgagactattgaaaaaccaaacaagaagctattctatcctgtgactatacctactcaactgaaatccgttcacgtgaaaccaatggggtcctctaccaaggtatcttcatctttgaaaatcaaggagttgaacaagaaggatgctgtaaagagatcgatcgaagaattgaagaatcagctggaatcgaaattataccgcgtgcgctcgtatttagaggatgaggaagtggttgaaaaagggccagcatcacaagttgaggctttgtcaacactggttgctgagaatcttgagtggttggactatgatagcgacgatgcatcagcaaaagatatcagggaaaaactaaattctgtgtcagatagtgttgccttcatcaagagctacattgatctgaacgatgtcacttttgataataatcttttcactacgatttacaacactactttaaactccatgcaaaatgttcaagaactaatgttaaacatgagtgaggatgctctgagtttaatgcagcagtatgagaaggaaggtttagacttcgccaaagaaagtcaaaagatcaaaataaaatctcctcctttatcagacaaagagcttgataatctctttaacactgttaccgaaaagttagagcatgtcagaatgttgactgaaaaggacactataagtgatttgcctagagaggagctttttaagctgtatcaagaattgcagaactactcttcccgatttgaagcaatcatggccagtttggaagatgtacactctcaaagaatcaaccgtttgacagacaagttacgcaaacatattgaaagggtgagcaatgaagcattgaaggcagctctcaaggaagctaaacgtcaacaagaggaggaaaaaagccacgagcagaatgagggagaagagcaaagttctgcttccacttctcacactaatgaagatatagaggaaccatcagaatcgcctaaggttcaaacatcccatgatgagttgtaagcttggagaccaatgac

SEQ ID NO: 114 - DNA, 42 bp, Artificial sequence
  oligonucleotide primer PP7435_Chr1-0059
gatctaggtctcccatgagaacacaaaagatagtaacagtac

SEQ ID NO: 115 - DNA, 40 bp, Artificial sequence
  oligonucleotide primer PP7435_Chr1-0059 reverse
gtcattggtctccaagcttacaactcatcatgggatgttt

SEQ ID NO: 116 - DNA, 1150 bp, Pichia pastoris
gatctaggtctcccatgaaagtgacattatctgtgttagctattgcctcccaattggttagaatcgtttgttcggaaggagaaaatatctgcataggtgaccagtgctatccgaagaattttgaacctgacaaggagtggaaacctgttcaggaaggccagattatccctccaggatcacacgtaagaatggactttaatacacaccagagagaggcaaaactggtggaagagaatgaggatatagacccctcatcattgggagtggctgtagtggattccaccggttcgtttgctgatgatcaatctttggaaaagattgagggactttccatggaacaactagatgagaagttagaagaactgattgagctttcccatgactacgagtacggatcagacataatcttgagtgatcagtatatttttggagtagccgggctagttcctactaagacaaagtttacttctgagttgaaggaaaaggccttgagaattgtcggatcatgcttgagaaacaatgccgatgcggtagagaaactactgggaactgttccaaatactataaccatacaattcatgtcaaacctagtgggtaaagtaaattccactggagagaatgttgactctgttgaacagaaacgaatcctttcaattattggagctgttattcctttcaaaattggaaaggtattgtttgaagcttgttcgggaacgcagaagctattactatccttggataaactggaaagttcagttcaactgagaggataccaaatgttggacgacttcattcatcaccctgaagaggaacttctctcttcattgacagcaaaggaacgattagtaaagcatattgagttgattcaatcattttttgcatcaggaaagcattctcttgatatagcaataaatcgtgagttattcactaggctgattgccttacgaaccaatttagaatctgccaatccaaatctatgtaaaccatcaactgactttttgaactggctgatcgacgaaattgaagctacgaaagataccgatccacacttttcaaaagagcttaaacatttacgttttgaactttttgggaacccattggcatctaggaaaggtttctccgatgagttataagcttggagaccaatgac

SEQ ID NO: 117 - DNA, 40 bp, Artificial sequence
  oligonucleotide primer PP7435_Chr1-0550
gatctaggtctcccatgaaagtgacattatctgtgttagc

SEQ ID NO: 118 - DNA, 42 bp, Artificial sequence
  oligonucleotide primer PP7435_Chr1-0550 reverse
gtcattggtctccaagcttataactcatcggagaaacctttc

SEQ ID NO: 119 - DNA, 931 bp, Pichia pastoris
gatctaggtctcccatgaaactacaccttgtgattctctgtttgatcactgctgtctactgtttcagtgctgttgacagagaaatctttcagctcaaccatgaattacgccaggaatacggagataattttaatttctatgaatggttgaagcttccaaaaggtccctcgtccacgtttgaagatatcgacaacgcgtacaagaaactatcccgtaagttacaccccgataagataagacagaagaaactatcccaggaacaatttgagcaattgaagaaaaaggctaccgaaagataccaacaattgagtgctgtgggatccatcttaagatccgagagcaaagagcgttacgattattttgtcaaacatggattcccagtctataaaggtaacgattacacctatgccaagtttagaccatccgttttgctcacaattttcatcctttttgcgttagctacgttaacccactttgtctttatcagattgtcggccgtgcaatctagaaaaagactgagttcgttgatagaggagaacaaacagctggcttggccacaaggtgttcaagatgtcactcaagtgaaggacgtcaaagtctataacgaacatctacgtaaatggtttttggtatgtttcgacggatccgttcattatgtggagaacgataaaaccttccatgttgatccggaagaagttgaactcccatcttggcaggacactcttccaggtaaattaatagtcaagctgataccccagcttgctagaaagccacgatctccaaaggagatcaagaaggaaaatttagatgataaaaccagaaagacaaaaaaacctacaggggattccaaaactttacctaacggtaaaaccatttataaagctaccaaatccggtggacgtagaaggaaataagcttggagaccaatgac

SEQ ID NO: 120 - DNA, 38 bp, Artificial sequence
  oligonucleotide primer PP7435_Chr1-0136
gatctaggtctcccatgaaactacaccttgtgattctc

SEQ ID NO: 121 - DNA, 38 bp, Artificial sequence
  oligonucleotide primer PP7435_Chr1-0136 reverse
gtcattggtctccaagcttatttccttctacgtccacc

(19) United States
(12) Patent Application Publication
(10) Pub. No.: US 2021/0269811 A1
(43) Pub. Date: Sep. 2, 2021

(54) MEANS AND METHODS FOR INCREASED PROTEIN EXPRESSION BY USE OF TRANSCRIPTION FACTORS

(71) Applicants: Boehringer Ingelheim RCV GmbH & CO KG, Wien (AT); Validogen GmbH, Grambach (AT); Lonza Ltd, Visp (CH)

(72) Inventors: Richard Zahrl, Wien (AT); Jonas Burgard, Wien (AT); Kristin Baumann, Esporles (ES); Diethard Mattanovich, Wien (AT); Brigitte Gasser, Wien (AT)

(21) Appl. No.: 17/254,238

(22) PCT Filed: Jun. 27, 2019
(86) PCT No.: PCT/EP2019/067133

§ 371 Date: Dec. 18, 2020

(30) Foreign Application Priority Data
Jun. 27, 2018 (EP) 18180164.8

(51) Int. Cl. C12N 15/81; C07K 16/00

(52) U.S. Cl. CPC C12N 15/815; C07K 2317/569; C07K 2317/622; C07K 16/00

(57) ABSTRACT

The present invention is in the field of recombinant biotechnology, in particular in the field of protein expression. The invention generally relates to a method of increasing the yield of a protein of interest (POI) in a eukaryotic host cell, preferably a yeast, by overexpressing at least one polynucleotide encoding at least one transcription factor of the present invention, preferably Msn4/2. The invention relates further to a recombinant eukaryotic host cell for manufacturing a POI, wherein the host cell is engineered to overexpress at least one polynucleotide encoding at least one transcription factor as well as the use of the host cell for manufacturing a POI.

### CROSS-REFERENCE TO RELATED APPLICATIONS

The present application claims the benefit of priority of EP Patent Application No. 18 180 164.8 filed 27 Jun. 2018, the content of which is hereby incorporated by reference in its entirety for all purposes.

### FIELD OF THE INVENTION

The present invention is in the field of recombinant biotechnology, in particular in the field of protein expression. The invention generally relates to a method of increasing the yield of a protein of interest (P01) in a eukaryotic host cell, preferably a yeast, by overexpressing at least one polynucleotide encoding at least one transcription factor of the present invention, preferably Msn4/2. The invention relates further to a recombinant eukaryotic host cell for manufacturing a P01, wherein the host cell is engineered to overexpress at least one polynucleotide encoding at least one transcription factor as well as the use of the host cell for manufacturing a P01.

### BACKGROUND OF THE INVENTION

Successful production of proteins of interest (P01) has been accomplished both with prokaryotic and eukaryotic hosts. The most prominent examples are bacteria like Escherichia coli, yeasts like Saccharomyces cerevisiae, Pichia pastoris or Hansenula polymorpha, filamentous fungi like Aspergillus awamori or Trichoderma reesei, or mammalian cells like CHO cells. While the yield of some proteins is readily achieved at high rates, many other proteins are only produced at comparatively low levels.

Generally, heterologous protein synthesis may be limited at different levels. Potential limits are transcription and translation, protein folding and, if applicable, secretion, disulfide bridge formation and glycosylation, as well as aggregation and degradation of the target proteins. Transcription can be enhanced by utilizing strong promoters or increasing the copy number of the heterologous gene. However, these measures clearly reach a plateau, indicating that other bottlenecks downstream of transcription limit expression.

High level of protein yield in host cells may also be limited at one or more different steps, like folding, disulfide bond formation, glycosylation, transport within the cell, or release from the cell. Many of the mechanisms involved are still not fully understood and cannot be predicted on the basis of the current knowledge of the state-of-the-art, even when the DNA sequence of the entire genome of a host organism is available. Moreover, the phenotype of cells producing recombinant proteins in high yields can be decreased growth rate, decreased biomass formation and overall decreased cell fitness.

Various attempts were made in the art for improving production of a protein of interest, such as overexpressing chaperones which should facilitate protein folding, external supplementation of amino acids, and the like.

However, there is still a need for methods to improve a host cell's capacity to produce and/or secrete proteins of interest. The technical problem underlying the present invention is to comply with this need.

The solution of the technical problem is the provision of means, such as engineered host cells, methods and uses applying said means for increasing the yield of a recombinant protein of interest in a eukaryotic host cell by overexpressing in said host cell at least one polynucleotide encoding at least one transcription factor. These means, methods and uses are described in detail herein, set out in the claims, exemplified in the Examples and illustrated in the Figures.

Accordingly, the present invention provides new methods and uses to increase the yield of recombinant proteins in host cells which are simple and efficient and suitable for use in industrial methods. The present invention also provides host cells to achieve this purpose.

It must be noted that as used herein, the singular forms “a”, “an” and “the” include plural references and vice versa unless the context clearly indicates otherwise. Thus, for example, a reference to “a host cell” or “a method” includes one or more of such host cells or methods, respectively, and a reference to “the method” includes equivalent steps and methods that could be modified or substituted known to those of ordinary skill in the art. Similarly, for example, a reference to “methods” or “host cells” includes “a host cell” or “a method”, respectively.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”. For example, A, B and/or C means A, B, C, A+B, A+C, B+C and A+B+C.

The term “about” or “approximately” as used herein means within 20%, preferably within 10%, and more preferably within 5% of a given value or range. It includes also the concrete number, e.g., about 20 includes 20.

The term “less than”, “more than” or “larger than” includes the concrete number. For example, less than 20 means 20 and more than 20 means 20.

Throughout this specification and the claims or items, unless the context requires otherwise, the word “comprise” and variations such as “comprises” and “comprising” will be understood to imply the inclusion of a stated integer (or step) or group of integers (or steps). It does not exclude any other integer (or step) or group of integers (or steps). When used herein, the term “comprising” can be substituted with “containing”, “composed of”, “including”, “having” or “carrying” and vice versa, by way of example the term “having” can be substituted with the term “comprising”. When used herein, “consisting of” excludes any integer or step not specified in the claim/item. When used herein, “consisting essentially of” does not exclude integers or steps that do not materially affect the basic and novel characteristics of the claim/item.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

It should be understood that this invention is not limited to the particular methodology, protocols, material, reagents, and substances, etc., described herein. The terminologies used herein are for the purpose of describing particular embodiments only and are not intended to limit the scope of the present invention, which is defined solely by the claims/items.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### SUMMARY

The findings of the present inventors are rather surprising, since the transcription factor of the present invention was to the best of one's knowledge up to the present invention not brought in connection with increasing the yield of a protein of interest in a eukaryotic host cell, particularly in a fungal host cell.

The present invention comprises a method of increasing the yield of a recombinant protein of interest in a eukaryotic host cell, comprising overexpressing in said host cell at least one polynucleotide encoding at least one transcription factor, thereby increasing the yield of said recombinant protein of interest in comparison to a host cell which does not overexpress the polynucleotide encoding said transcription factor, wherein the transcription factor comprises at least: a) a DNA binding domain comprising: i) an amino acid sequence as shown in SEQ ID NO: 1, or ii) a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87, and b) an activation domain.

The method of the present invention may comprise:
i) engineering the host cell to overexpress at least one polynucleotide encoding at least one transcription factor comprising at least:
   a) a DNA binding domain comprising:
      a1) an amino acid sequence as shown in SEQ ID NO: 1, or
      a2) a functional homolog of the amino acid sequence as shown in SEQ ID NO: 1 having at least 60% sequence identity to the amino acid sequence as shown in SEQ ID NO: 1 and/or having at least 60%, sequence identity to an amino acid sequence as shown in SEQ ID NO: 87, and
   b) an activation domain,
ii) engineering said host cell to comprise a polynucleotide encoding the protein of interest,
iii) culturing said host cell under suitable conditions to overexpress the at least one polynucleotide encoding at least one transcription factor and to overexpress the protein of interest, optionally
iv) isolating the protein of interest from the cell culture, and optionally
v) purifying the protein of interest.

Additionally, the present invention envisages a method of manufacturing a recombinant protein of interest by a eukaryotic host cell comprising:
i) providing the host cell engineered to overexpress at least one polynucleotide encoding at least one transcription factor, wherein the host cell further comprises a polynucleotide encoding a protein of interest, wherein the transcription factor comprises at least:
   a) a DNA binding domain comprising:
      a1) an amino acid sequence as shown in SEQ ID NO: 1, or
      a2) a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87, and
   b) an activation domain,
ii) culturing said host cell under suitable conditions to overexpress the at least one polynucleotide encoding at least one transcription factor and to overexpress the protein of interest, optionally
iii) isolating the protein of interest from the cell culture, and optionally
iv) purifying the protein of interest, and optionally
v) modifying the protein of interest, and optionally
vi) formulating the protein of interest.

The method of the present invention may comprise that overexpression of said transcription factor increases the yield of the model protein scFv (SEQ ID NO. 13) and/or vHH (SEQ ID NO. 14) compared to the host cell prior to engineering.

Further, the present invention may comprise the method of the present invention, wherein the polynucleotide encoding the at least one transcription factor is integrated in the genome of said host cell or contained in a vector or plasmid, which does not integrate into the genome of said host cell.

The present invention may encompass the method of the present invention, wherein the eukaryotic host cell is a fungal host cell, preferably a yeast host cell selected from the group consisting of Pichia pastoris (syn. Komagataella spp), Hansenula polymorpha (syn. H. angusta), Trichoderma reesei, Aspergillus niger, Saccharomyces cerevisiae, Kluyveromyces lactis, Yarrowia lipolytica, Pichia methanolica, Candida boidinii, Komagataella spp and Schizosaccharomyces pombe. Hansenula polymorpha has been reclassified to the genus Ogataea (Yamada et al. 1994. Biosci Biotechnol Biochem. 58(7):1245-57). Ogataea angusta, Ogataea polymorpha and Ogataea parapolymorpha are closely related species, that have been separated from each rather recently (Kurtzman et al. 2011. Antonie Van Leeuwenhoek. 100(3):455-62).

The present invention may envisage the method of the present invention, wherein the recombinant protein of interest is an enzyme, a therapeutic protein, a food additive or feed additive.

Additionally, the present invention may comprise the method of the present invention, further comprising overexpressing in said host cell or engineering said host cell to overexpress at least one polynucleotide encoding at least one ER helper protein.

Preferably, said ER helper protein has an amino acid sequence as shown in SEQ ID NO: 28 or a functional homolog thereof having at least 70% sequence identity to an amino acid sequence as shown in SEQ ID NO: 28.

Contemplated by the present invention may be the method of the present invention, further comprising overexpressing in said host cell or engineering said host cell to overexpress at least two polynucleotides encoding at least two ER helper proteins.

Preferably, the first ER helper protein has an amino acid sequence as shown in SEQ ID NO: 28 or a functional homologue thereof having at least 70% sequence identity to the amino acid sequence as shown in SEQ ID NO: 28, and the second ER helper protein may have an amino acid sequence:
i) as shown in SEQ ID NO: 37, or a functional homologue thereof having at least 25% sequence identity to the amino acid sequence as shown in SEQ ID NO: 37, or
ii) as shown in SEQ ID NO. 47, or a homologue thereof, wherein the homologue has at least 20% sequence identity to the amino acid sequence as shown in SEQ ID NO. 47.
   Optionally, the third ER helper protein may have an amino acid sequence as shown in SEQ ID NO: 55, or a functional homologue thereof having at least 25% sequence identity to the amino acid sequence as shown in SEQ ID NO: 55.

Additionally, the present invention may comprise the method of the present invention, further comprising overexpressing in said host cell or engineering said host cell to overexpress at least one polynucleotide encoding one additional transcription factor.

Preferably, the additional transcription factor comprises at least:
a) a DNA binding domain comprising:
   i) an amino acid sequence as shown in SEQ ID NO: 65, or
   ii) a functional homolog of the amino acid sequences as shown in SEQ ID NO: 65 having at least 50% sequence identity to an amino acid sequence as shown in SEQ ID NO: 65, and
b) an activation domain.

The present invention also comprises a recombinant eukaryotic host cell for manufacturing a protein of interest, wherein the host cell is engineered to overexpress at least one polynucleotide encoding at least one transcription factor, wherein the transcription factor comprises at least:
a) a DNA binding domain comprising:
   i) an amino acid sequence as shown in SEQ ID NO: 1, or
   ii) a functional homolog of the amino acid sequence as shown in SEQ ID NO: 1 having at least 60% sequence identity to the amino acid sequence as shown in SEQ ID NO: 1 and/or having at least 60% identity to an amino acid sequence as shown in SEQ ID NO: 87, and
b) an activation domain.

Contemplated by the present invention is also the use of the recombinant eukaryotic host cell as mentioned above for manufacturing a recombinant protein of interest.

### BRIEF DESCRIPTION OF THE DRAWINGS

FIG. 1: Improvement of vHH secretion (titer and yield) in small scale screening cultures.

Overview of overexpressed genes or gene combinations that increase vHH secretion in P. pastoris in small scale screening. The plasmid or plasmids used for engineering the host cell to overexpress these genes or gene combinations are shown below the genes or gene combinations in brackets. The fold-change values of small scale screenings are an arithmetic mean of up to 20 clones/transformants.

FIG. 2: Improvement of vHH secretion (titer and yield) in fed batch bioreactor cultivations.

Overview of overexpressed genes or gene combinations that increase vHH secretion in P. pastoris in fed batch cultivations. The plasmid or plasmids used for engineering the host cell to overexpress these genes or gene combinations are shown below the genes or gene combinations in brackets. The fold-change values of fed batch cultivations are those of the single selected clone.

FIG. 3: Improvement of scFv secretion (titer and yield) in small scale screening cultures.

Overview of overexpressed genes or gene combinations that increase scFv secretion in P. pastoris in small scale screening. The plasmid or plasmids used for engineering the host cell to overexpress these genes or gene combinations are shown below the genes or gene combinations in brackets. The fold-change values of small scale screenings are an arithmetic mean of up to 20 clones/transformants.

FIG. 4: Improvement of scFv secretion (titer and yield) in fed batch bioreactor cultivations.

Overview of overexpressed genes or gene combinations that increase scFv secretion in P. pastoris in fed batch cultivations. The plasmid or plasmids used for engineering the host cell to overexpress these genes or gene combinations are shown below the genes or gene combinations in brackets. The fold-change values of fed batch cultivations are those of the single selected clone.

FIG. 5: Improvement of scFv secretion (titer and yield) by overexpression of MSN2/4 homologs from other species in fed batch bioreactor cultivations.

FIG. 6: Overview of alignment of different derived Msn4p transcription factors.

The protein structural motif of the zinc finger shows clearly a strong conservation (box in FIG. 6), which is known as the DNA binding domain of the well characterized transcription factor Msn4p and Msn2p in S. cerevisiae (ScMsn4/2).

FIG. 7: The amino acid consensus sequence of the Msn4-like C₂H₂ zinc finger DNA binding domain.

FIG. 8: Sequence alignments of P. pastoris MSN4/2.

Pairwise sequence similarities/identities between the full length Msn4p of P. pastoris and each homolog of the other organisms was assessed by a global pairwise sequence alignment with the EMBOSS Needle algorithm. Pairwise sequence similarities/identities were also investigated for the DNA-binding domain of Msn4p of P. pastoris and the DNA-binding domains of each homolog of the other organisms.

FIG. 9: Sequence identity to P. pastoris KAR2.

Sequence identity was assessed with BLASTp.

FIG. 10: Sequence identity to P. pastoris LHS1.

Sequence identity was assessed with BLASTp.

FIG. 11: Sequence identity to P. pastoris SIL1.

Sequence identity was assessed with BLASTp.

FIG. 12: Sequence identity to P. pastoris ERJ5.

Sequence identity was assessed with BLASTp.

FIG. 13: Sequence alignments of P. pastoris HAC1.

Pairwise sequence similarities/identities between the full length Hac1p of P. pastoris and each homolog of the other organisms was assessed by a global pairwise sequence alignment with the EMBOSS Needle algorithm. Pairwise sequence similarities/identities were also investigated for the DNA-binding domain of Hac1p of P. pastoris and the DNA-binding domains of each homolog of the other organisms.

FIG. 14: Sequence identity to the consensus sequence of the MSN4/2-DNA binding domain.

Pairwise sequence similarities/identities were investigated between the consensus sequence of the DNA-binding domain (DBD) of Msn4p/Msn2p and the DNA-binding domains of each homolog of the other organisms by a global pairwise sequence alignment with the EMBOSS Needle algorithm.

### DETAILED DESCRIPTION OF THE INVENTION

The present invention is partly based on the surprising finding of the overexpression of the at least one transcription factor as described herein, which was found to increase the yield of a recombinant protein of interest. In particular, the present invention comprises a method of increasing the yield of a recombinant protein of interest in a eukaryotic host cell, comprising overexpressing in said host cell at least one polynucleotide encoding at least one transcription factor of the present invention, thereby increasing the yield of said recombinant protein of interest in comparison to a host cell which does not overexpress the polynucleotide encoding said transcription factor.

The term “increasing the yield of a recombinant protein of interest in a host cell” means that the yield of the protein of interest (P01) is increased when compared to the same cell expressing the same POI under the same culturing conditions, however, without the polynucleotide encoding the transcription factor being overexpressed or without being engineered to overexpress the polynucleotide encoding the transcription factor.

In this context the term “yield” refers to the amount of POI or model protein(s) as described herein, in particular scFv, a single chain variable fragment (SEQ ID NO: 13) and vHH (or VHHV), a single-domain antibody fragment (SEQ ID NO. 14) respectively, which is, for example, harvested from the engineered host cell, and increased yields can be due to increased amounts of production inside the host cell or the increased secretion of the POI by the host cell. The term “yield” also refers to the amount of POI or model protein(s) as described herein per cell and may be presented by mg POI/g biomass (measured as dry cell weight or wet cell weight) of a host cell. The term “titer” when used herein refers similarly to the amount of produced POI or model protein, presented as mg POI/L culture supernatant or whole cell broth. The present invention may also comprise a method of increasing the titer of a recombinant protein of interest, wherein the transcription factor of the present invention is overexpressed in a eukaryotic host cell. An increase in yield can be determined when the yield obtained from an engineered host cell is compared to the yield obtained from a host cell prior to engineering, i.e., from a non-engineered host cell. Preferably, “yield” when used herein in the context of a model protein as described herein, is determined as described in Examples 3, 4 and 5. For example, the term “yield” may refer to the amount of POI that is produced by a certain amount of biomass throughout a submersion cultivation. Therein, the recombinant POI can be produced and accumulated inside the cell or be secreted to the culture supernatant. The term “increasing the yield of a recombinant protein of interest in a host cell” refers to increasing the amount of POI produced within the or by the cell and/or to increasing the amount of POI secreted from the cell.

As will be appreciated by a skilled person in the art, the overexpression of the transcription factor of the present invention has been shown to increase the yield as well as increase the titer of POI, in particular of a recombinant POI.

The term “protein of interest” (P01) as used herein generally relates to any protein but preferably relates to a “heterologous protein” or “recombinant protein”, preferably the model proteins scFv (SEQ ID NO: 13) and/or vHH (SEQ ID NO. 14). Specific examples of the POI of the present invention are indicated elsewhere herein. As used herein, “recombinant” refers to the alteration of genetic material by human intervention. Typically, recombinant refers to the manipulation of DNA or RNA in a virus, cell, plasmid or vector by molecular biology (recombinant DNA technology) methods, including cloning and recombination. A recombinant protein can be typically described with reference to how it differs from a naturally occurring counterpart (the “wild-type”). Preferably, the recombinant protein of interest expressed by the eukaryotic host cell of the present invention is from a different organism. The POI is preferably not a transcription factor, i.e. the transcription factor and the POI are not identical. A recombinant protein also may be a homologous protein. In this case one or more copies of the polynucleotide encoding the homologous protein are introduced into the host cell by genetic manipulation.

The term “expressing a polynucleotide” means when a polynucleotide is transcribed to mRNA and the mRNA is translated to a polypeptide. The term “overexpress” generally refers to any amount greater than an expression level exhibited by a reference standard (e.g., the same host cell under the same culturing conditions, which is not engineered to overexpress a polynucleotide encoding a protein). The terms “overexpress,” “overexpressing,” “overexpressed” and “overexpression” in the present invention refer to an expression of a gene product or a polypeptide at a level greater than the expression of the same gene product or polypeptide prior to a genetic alteration of the host cell or in a comparable host which has not been genetically altered at defined conditions. In the present invention, a transcription factor comprising an amino acid sequence as shown in any one of SEQ ID NOs: 15-27 or a functional homolog thereof is overexpressed. If a host cell does not comprise a given gene product, it is possible to introduce the gene product into the host cell for expression; in this case, any detectable expression is encompassed by the term “overexpression.” In preferred embodiments, “overexpressing” means “engineering to overexpress” as described below. Such preferred embodiments are contemplated for any embodiment relating to “overexpression” or “overexpressing” as described herein.

A “polynucleotide” as used herein, refers to nucleotides, either ribonucleotides or deoxyribonucleotides or a combination of both, in a polymeric unbranched form of any length. Preferably, a polynucleotide refers to deoxyribonucleotides in a polymeric unbranched form of any length. Here, nucleotides consist of a pentose sugar (deoxyribose), a nitrogenous base (adenine, guanine, cytosine or thymine) and a phosphate group. The terms “polynucleotide(s)”, “nucleic acid sequence(s)” are used interchangeably herein.

As used herein, the term “at least one polynucleotide encoding at least one transcription factor” refers to one polynucleotide encoding one transcription factor, two polynucleotides encoding two transcription factors, three polynucleotide encoding three transcription factors, four polynucleotides encoding four transcription factors etc. Preferably, one polynucleotide encoding one transcription factor is comprised by the present invention. More preferably, one polynucleotide encoding one transcription factor and one polynucleotide encoding one additional transcription factor is comprised by the present invention.

The term “transcription factor” refers to a protein that controls the rate of transcription of genetic information from DNA to messenger RNA, by binding to a specific DNA sequence, preferably with its DNA binding domain. Their function is to regulate—and/or activate genes in order to make sure that they are expressed in the right cell at the right time and in the right amount. For example, a transcription factor may initiate the transcription of a specific gene(s) in response to a stimulus, such as starvation or heat shock. In the present invention the Msn4p transcription factor refers to SEQ ID NO. 15-27 comprising a DNA binding domain and to transcription factors comprising an amino acid sequence as shown in SEQ ID NO: 1 or a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87 as described herein and any activation domain (e.g.: synthetic, viral or an activation domain of the transcription factor of the present invention or other transcription factors of any species as described elsewhere herein), preferably the activation domain as can be seen in SEQ ID NO. 83. The arrangement of said DNA binding domain of the transcription factor of the present invention as described herein and any activation domain may be performed according to the skilled person's knowledge and may be performed in any order. The DNA binding domain of the transcription factor of the present invention may be arranged by the skilled person C- or N-terminally, preferably C-terminally. In a further embodiment, a synthetic version of the transcription factor of the present invention (e.g.: synMSN4) may also be used in the present invention (such as SEQ ID NO. 27). A synthetic version of the transcription factor may comprise a synthetic DNA binding domain (such as SEQ ID NO. 12). Further, a synthetic version of the transcription factor of the present invention may comprise any activation domain (a synthetic, a viral or an activation domain of the transcription factor of the present invention or other transcription factors of any species as described elsewhere herein), preferably the activation domain as can be seen in SEQ ID NO. 84. Again the arrangement of said DNA binding domain of the transcription factor of the present invention as described herein and any activation domain may be performed according to the skilled person's knowledge and may be performed in any order. The DNA binding domain of the synthetic transcription factor of the present invention may be arranged by the skilled person C- or N-terminally, preferably C-terminally.

In the present invention the transcription factor refers to Msn4/2 protein (Msn4/2p or MSN4/2). Msn4p is a homolog to Msn2p in yeasts such as S. cerevisiae and its close relatives that underwent the whole genome duplication event. Most other yeast and fungal species only contain on Msn-type transcription factor, and there cannot be a reasonable distinction of these transcription factors in these species. Due to this functional redundancy, these transcription factors can be either addressed as Msn2 or Msn4 or Msn4/2. Due to the high homology, it is highly probable that Msn4p and Msn2p are interchangeable, i.e., that the transcription factors are redundant. There are no fundamental differences in Msn2- and Msn4-dependent expression, and also the structures of Msn4p and Msn2p are very similar. Pichia pastoris has only one homolog, named Msn4p. Also in several other yeasts, there is only a single homolog to Msn4/2, which may have different names. In Aspergillus niger, the homolog of Msn4/2 is called Seb1. In S. cerevisiae the homolog of Msn4/2 is called Com2.

MSN4 (such as MSN2) encodes transcription factors that regulate the general stress response. In S. cerevisiae, Msn4p (such as Msn2p) regulates the expression of ˜200 genes in response to several stresses, including heat shock, osmotic shock, oxidative stress, low pH, glucose starvation, sorbic acid and high ethanol concentrations, by binding to the STRE element, 5′-CCCCT-3′, located in the promoters of these genes by the Msn4p (such as Msn2p) zinc-finger binding domain at the C-terminus. In their N-terminus, Msn4p (such as Msn2p) contains a transcription-activating domain and a nuclear export sequence. Further, Msn4p (such as Msn2p) comprises a nuclear localization signal, which is inhibited by PKA phosphorylation and activated by protein phosphatase 1 dephosphorylation. Under non-stress conditions, Msn4p (such as Msn2p) is located in the cytoplasm. Cytoplasmic localization is partially regulated by TOR signalling. Upon stress, Msn4p (such as Msn2p) is hyperphosphorylated, relocalized to the nucleus and then displays a periodic nucleo-cytoplasmic shuttling behavior.

Preferably, the transcription factor of the present invention comprises an amino acid sequence as shown in SEQ ID NOs: 15-27.

Until now, it was nowhere to be found that the transcription factor Msn4p is involved in increasing the yield/titer of a recombinant POI, or in general involved in the secretion of a recombinant POI by a eukaryotic host cell. Thus, it was surprising that the overexpression of Msn4p in a eukaryotic host cell increased the yield/titer of a recombinant POI in the present invention.

In the present invention the transcription factor was originally isolated from Pichia pastoris (Komagataella phaffi) CBS7435 strain (CBS-KNAW culture collection). It is envisioned that the transcription factor can be overexpressed over a wide range of host cells. Thus, instead of using the sequences native to the species or the genus, the transcription factor sequences may also be taken or derived from other prokaryotic or eukaryotic organisms, preferably from fungal host cells, more preferably from a yeast host cell such as Pichia pastoris (syn. Komagataella spp), Hansenula polymorpha (syn. H. angusta), Trichoderma reesei, Aspergillus niger Saccharomyces cerevisiae, Kluyveromyces lactis, Yarrowia lipolytica, Pichia methanolica, Candida boidinii, Komagataella spp and Schizosaccharomyces pombe. Preferably, the transcription factor is derived from Pichia pastoris (Komagataella spp), Saccharomyces cerevisiae, Yarrowia lipolytica or Aspergillus niger, more preferably from Pichia pastoris (Komagataella spp). Further, a synthetic version of the transcription factor of the present invention may also be used. As used herein, Komagataella spp. comprises all species of the genus Komagataella. In preferred embodiments, the transcription factor is derived from Komagataella pastoris, Komagataella pseudopastoris or Komagataella phaffii. In an even more preferred embodiment, the transcription factor is derived from Komagataella pastoris or Komagataella phaffii.

Preferably, the transcription factor used in the methods, in the recombinant host cell and in the use of the recombinant host cell of the present invention comprises at least a DNA binding domain comprising an amino acid sequence as shown in SEQ ID NO: 1 (DNA binding domain of Msn4p of Pichia pastoris, in particular of Komagataella phaffi or Komagataella pastoris) and an activation domain. Thus, the method, the recombinant host cell and the use of the present invention preferably overexpress a transcription factor comprising at least a DNA binding domain comprising an amino acid sequence as shown in SEQ ID NO: 1 and an activation domain in Pichia pastoris (Komagataella spp). The overexpression of said transcription factor comprising at least a DNA binding domain comprising an amino acid sequence as shown in SEQ ID NO: 1 and an activation domain in Hansenula polymorpha, Trichoderma reesei, Aspergillus niger, Saccharomyces cerevisiae, Kluyveromyces lactis, Yarrowia lipolytica, Pichia methanolica, Candida boidinii, Komagataella spp, or Schizosaccharomyces pombe is also preferred.

The transcription factor used in the methods, in the recombinant host cell and in the use of the recombinant host cell of the present invention comprises at least a DNA binding domain comprising a functional homolog of the amino acid sequence as shown in SEQ ID NO: 1 (DNA binding domain of Msn4p of Pichia pastoris) having at least 60% sequence identity to the amino acid sequence as shown in SEQ ID NO: 1 and an activation domain. Additionally, the transcription factor used in the methods, in the recombinant host cell and in the use of the recombinant host cell of the present invention comprising at least a DNA binding domain comprising a functional homolog of the amino acid sequence as shown in SEQ ID NO: 1 (DNA binding domain of Msn4p of Pichia pastoris) having at least 60% sequence identity to an amino acid sequence as shown in SEQ ID NO: 87 and an activation domain is also contemplated by the present invention. Preferably, the transcription factor used in the methods, in the recombinant host cell and in the use of the recombinant host cell of the present invention comprises at least a DNA binding domain comprising a functional homolog of the amino acid sequence as shown in SEQ ID NO: 1 (DNA binding domain of Msn4p of Pichia pastoris) having at least 60% sequence identity to the amino acid sequence as shown in SEQ ID NO: 1 and/or having at least 60% sequence identity to an amino acid sequence as shown in SEQ ID NO: 87, and an activation domain. Thus, the method, the recombinant host cell and the use of the present invention may further comprise overexpressing a transcription factor comprising at least a DNA binding domain comprising a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87 and an activation domain in Pichia pastoris. Thus, the method, the recombinant host cell and the use of the present invention may further comprise overexpressing a transcription factor comprising at least a DNA binding domain comprising a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87 and an activation domain in Hansenula polymorpha, Trichoderma reesei, Aspergillus niger, Saccharomyces cerevisiae, Kluyveromyces lactis, Yarrowia lipolytica, Pichia methanolica, Candida boidinii, Komagataella spp, or Schizosaccharomyces pombe.

Preferably, the functional homologs of the amino acid sequence as shown in SEQ ID NO. 1 having at least 60% sequence identity to the amino acid sequence as shown in SEQ ID NO: 1 and/or having at least 60% sequence identity to an amino acid sequence as shown in SEQ ID NO: 87, have the amino acid sequences as shown in SEQ ID NOs: 2, 3, 4, 5, 6, 7, 8, 9, 10, 11 and 12.

Thus, the method, the recombinant host cell and the use of the present invention may further comprise overexpressing a transcription factor comprising at least a DNA binding domain comprising an amino acid sequence as shown in SEQ ID NOs: 1, 2, 3, 4, 5, 6, 7, 8, 9, 10, 11 and 12 and an activation domain.

Additionally, the method, the recombinant host cell and the use of the present invention may further encompass overexpressing a transcription factor comprising at least a DNA binding domain comprising an amino acid sequence as shown in SEQ ID NOs: 1, 2, 3, 4, 5, 6, 7, 8, 9, 10, 11 and 12 and an activation domain in Pichia pastoris. Thus, the method, the recombinant host cell and the use of the present invention may comprise overexpressing a transcription factor comprising at least a DNA binding domain comprising an amino acid sequence as shown in SEQ ID NOs: 1, 2, 3, 4, 5, 6, 7, 8, 9, 10, 11 and 12 and an activation domain in Hansenula polymorpha, Trichoderma reesei, Aspergillus niger, Saccharomyces cerevisiae, Kluyveromyces lactis, Yarrowia lipolytica, Pichia methanolica, Candida Komagataella spp., or Schizosaccharomyces pombe.

A “DNA binding domain” or “binding domain” as used herein refers to the domain of the transcription factor that binds to DNA of its regulated genes. Preferably, the DNA binding domain of the present invention is selected from the group consisting of SEQ ID NOs. 1 or a functional homolog of the amino acid sequence as shown in SEQ ID NO. 1 having at least 60% sequence identity to the amino acid sequence as shown in SEQ ID NO.1 and/or having at least 60% sequence identity to an amino acid sequence as shown in SEQ ID NO: 87 (such as SEQ ID NOs: 2, 3, 4, 5, 6, 7, 8, 9, 10, 11 and 12). Most preferred is the DNA binding domain as shown in SEQ ID NO. 1. Thus, the present invention may also comprise a synthetic DNA binding domain as can be seen from SEQ ID NO. 12.

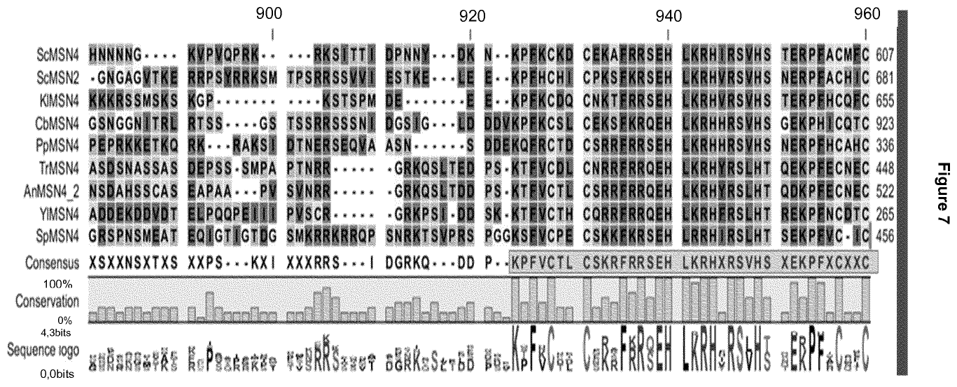

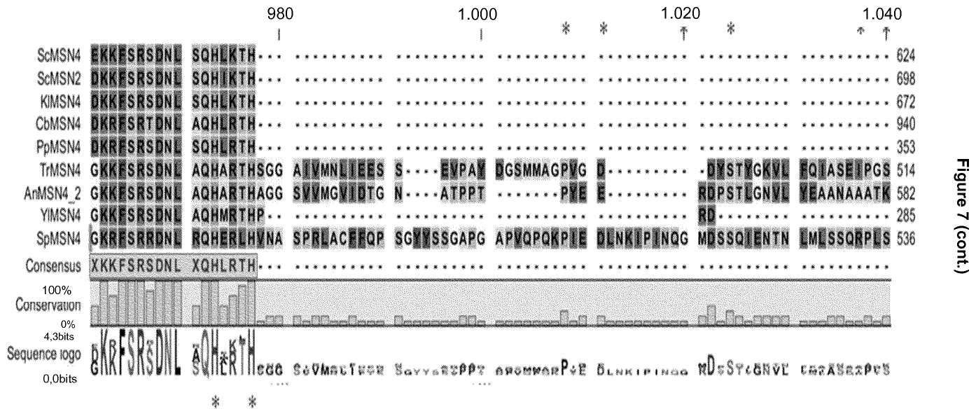

As used herein, the SEQ ID NO. 87 refers to the consensus sequence of the MSN4/2-like C₂H₂ type zinc finger DNA binding domain (see FIG. 6). The alignment of the different derived MSN4/2 transcription factors was performed with the software CLC Main Workbench (QIAGEN Bioinformatics) as described in Example 6. Here, the known DNA binding domain of Msn4p/Msn2p in S. cerevisiae, which is a model organism often used in experiments and which underwent a whole-genome duplication (WGD, thus having two homologs, Msn4p and Msn2p, is used to derive the same function in other organisms. The zinc finger in S. cerevisiae's Msn2/4 has a C₂H₂-like fold, having an amino acid sequence motif of X₂-C-X_{2,4}-C-X₁₂-H-X_{3,4,5}-H (see FIG. 7). The consensus sequence of the Msn4/2 DNA binding domain (SEQ ID NO: 87) has the following sequence:

KPFVCTLCSKRFRRXEHLKRHXRSXHSXEKPFXCXXCXKKFSRS
DNLXQHLRTH

whereby K at position 10 can be interchangeable with R;
R at position 11 can be interchangeable with K;
Xaa at position 15 can be Q or S;
K at position 19 can be interchangeable with R;
Xaa at position 22 can be any naturally occurring amino acid;
Xaa at position 25 can be V or L;
S at position 27 can be interchangeable with T;
Xaa at position 28 can be any naturally occurring amino acid;
K at position 30 can be interchangeable with R;
Xaa at position 33 can be any naturally occurring amino acid;
Xaa at position 35-36 can be any naturally occurring amino acid;
Xaa at position 38 can be any naturally occurring amino acid;
K at position 40 can be interchangeable with R;
S at position 44 can be interchangeable with T;
Xaa at position 48 can be any naturally occurring amino acid;
R at position 52 can be interchangeable with K.
Bold letters are highly conserved, underlined letters are part of the C₂H₂ type zinc finger.

As used herein, a “homologue” or “homolog” of the transcription factor or the binding domain of the transcription factor of the present invention shall mean that a protein has the same or conserved residues at a corresponding position in their primary, secondary or tertiary structure. The term also extends to two or more nucleotide sequences encoding homologous polypeptides. When the function as a transcription factor or as a binding domain of the transcription factor is proven with such a homologue, the homologue is called “functional homologue”. A functional homologue performs the same or substantially the same function as the transcription factor or the binding domain of the transcription factor from which it is derived from. In the case of nucleotide sequences a “functional homologue” preferably means a nucleotide sequence having a sequence different form the original nucleotide sequence, but which still codes for the same amino acid sequence, due to the use of the degenerated genetic code. Functional homologs of a protein in particular the transcription factor or the binding domain of the transcription factor may be obtained by substituting one or more amino acids of the protein in particular the transcription factor or the binding domain of the transcription factor, whose substitution(s) preserve the function of the protein in particular the transcription factor or the binding domain of the transcription factor. In particular, a functional homolog of the amino acid sequence as shown in SEQ ID NO: 1 has at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 1 (DNA binding domain of Msn4p of Pichia pastoris) and/or at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 60% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 61% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 62% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 63% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 64% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 65% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 66% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 67% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 68% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 69% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 70% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 71% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 72% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 73% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 74% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 75% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 76% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 77% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 78% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 79% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 80% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 81% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 82% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 83% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 84% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 85% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 86% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 87% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 88% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 89% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 90% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 91% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 92% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 93% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 94% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 95% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 96% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 97% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 98% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has at least about 99% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence). In some embodiments, a functional homolog of the amino acid sequence as shown in SEQ ID NO: 1 has about 100% amino acid sequence identity to the amino acid sequence as shown in SEQ ID NO: 1 (DNA binding domain of Msn4p of Pichia pastoris) and at least about 60%, such as at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% amino acid sequence identity to the amino acid sequence as shown in SEQ ID NO: 87 (consensus sequence).

Generally, homologues can be prepared using any mutagenesis procedure known in the art, such as site-directed mutagenesis, synthetic gene construction, semi-synthetic gene construction, random mutagenesis, shuffling, etc. Site-directed mutagenesis is a technique in which one or more (e.g., several) mutations are introduced at one or more defined sites in a polynucleotide encoding the parent. Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et ai, 1990, Nucleic Acids Res. 18: 7349-4966. Site-directed mutagenesis can also be accomplished in vivo by methods known in the art. See, e.g., U.S. Patent Application Publication No. 2004/0171 154; Storici et ai, 2001, Nature Biotechnol. 19: 773-776; Kren et ai, 1998, Nat. Med. 4: 285-290; and Calissano and Macino, 1996, Fungal Genet. Newslett. 43: 15-16. Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips. Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al, 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7:127). Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods known in the art. These methods allow the rapid determination of the importance of individual amino acid residues in a polypeptide. Semi-synthetic gene construction is accomplished by combining aspects of synthetic gene construction, and/or site-directed mutagenesis, and/or random mutagenesis, and/or shuffling. Semisynthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled. Alternatively, homologues for example can be obtained from a natural source such as by screening cDNA libraries of other organisms, or by homology searches in nucleic acid databases, preferably homologues of closely related or related organisms such as Komagataella pastoris, Komagataella pseudopastoris or Komagataella phaffii, Komagatella spp, Hansenula polymorpha, Trichoderma reesei, Aspergillus niger, Saccharomyces cerevisiae, Kluyveromyces lactis, Yarrowia lipolytica, Pichia methanolica, Candida boidinii, Komagataella spp., or Schizosaccharomyces pombe. Thus, SEQ ID NOs.: 2-12 are functional homologs of the binding domain of the transcription factor as shown in SEQ ID NO:1 and SEQ ID NOs.: 16-27 are functional homologs of the transcription factor as shown in SEQ ID NO 15.

The function of a homologue of the amino acid sequence of the DNA-binding domain as shown in SEQ ID NO: 1 having at least 60% sequence identity to the amino acid sequence as shown in SEQ ID NO. 1 (such as SEQ ID NOs: 2, 3, 4, 5, 6, 7, 8, 9, 10, 11 and 12) and/or having at least 60% sequence identity to an amino acid sequence as shown in SEQ ID NO: 87 or the function of a homologue of the amino acid sequence of the transcription factor as shown in SEQ ID NO. 15 having at least 11% sequence identity to the amino acid sequence as shown in SEQ ID NO. 15 (such as SEQ ID Nos: 16, 17, 18, 19, 20, 21, 22, 23, 24, 25, 26, 27) or the function of a homologue of the amino acid sequence of the DNA-binding domain of the additional transcription factor as shown in SEQ ID NO: 65 having at least 50% sequence identity to an amino acid sequence as shown in SEQ ID NO. 65 (such as SEQ ID NOs: 66-73) or the function of a homologue of the amino acid sequence of the additional transcription factor as shown in SEQ ID NO. 74 having at least 20% sequence identity to the amino acid sequence as shown in SEQ ID NO. 74 (such as SEQ ID Nos: 75, 76, 77, 78, 79, 80, 81, 82) as disclosed herein can be tested by providing expression cassettes into which the transcription factor comprising the homologues of the amino acid sequence of the DNA-binding domain as shown in SEQ ID NO: 1 and an activation domain (e.g.: SEQ ID NO: 83 or 84 or the like) and a nuclear localization signal (NLS) (e.g.: SEQ ID NO: 85 or 86 or the like) or the additional transcription factor comprising the homologues of the amino acid sequence of the DNA-binding domain as shown in SEQ ID NO: 65 and an activation domain and a nuclear localization signal (NLS) or the homologues of the amino acid sequence of the transcription factor as shown in SEQ ID NO. 15 or the homologues of the amino acid sequence of the transcription factor as shown in SEQ ID NO. 74 have been inserted, transforming host cells that carry the sequence encoding a test protein such as one of the model proteins used in the Example section or another POI, and determining the difference in the yield of the model protein or POI under identical conditions.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

“Sequence identity” or “% identity” refers to the percentage of residue matches between at least two polypeptides or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. The sequence identity used in the present invention refers to the percentage of having identical amino acids between at least two polypeptide sequences (amino acid sequences). The sequence similarity listed in the present invention refers to the percentage of having similar amino acids being group according to their side chains and charges between at least two polypeptide sequences (amino acid sequences). For purposes of the present invention, the sequence identity between two amino acid sequences or nucleotide sequences is determined using the NCBI BLAST program version 2.2.29 (Jan. 6, 2014) (Altschul et al., Nucleic Acids Res. (1997) 25:3389-3402). Sequence identity of two amino acid sequences can be determined with blastp set at the following parameters: Matrix: BLOSUM62, Word Size: 3; Expect value: 10; Gap cost: Existence=11, Extension=1; Filter=low complexity deactivated; Compositional adjustments: Conditional compositional score matrix adjustment. For purposes of the present invention, the sequence identity between two nucleotide sequences is determined using the NCBI BLAST program version 2.2.29 (Jan. 6, 2014) with blastn set at the following exemplary parameters: Word Size: 28; Expect value: 10; Gap costs: Linear; Filter=low complexity activated; Match/Mismatch Scores: 1,−2. For purposes of the present invention, the sequence identity between two amino acid sequences or nucleotide sequences is further determined using BLAST and EMBOSS Needle algorithm. The sequence identity for the DNA binding domain was assessed by said global pairwise sequence alignment with the EMBOSS Needle algorithm. The EMBOSS Needle webserver (https://www.ebi.ac.uk/Tools/psa/emboss_needle/) was used for pairwise protein sequence alignment using default settings (Matrix: BLOSUM62; Gap open:10; Gap extend: 0.5; End Gap Penalty: false; End Gap Open: 10; End Gap Extend: 0.5). EMBOSS Needle reads two input sequences and writes their optimal global sequence alignment to file. It uses the Needleman-Wunsch alignment algorithm to find the optimum alignment (including gaps) of two sequences along their entire length. The sequence identity to P. pastoris KAR2, LHS1, SIL1 and ERJ5 was determined by BLAST.

As used herein, the term “activation domain” refers to any domain capable of activating transcription. As an activation domain each activation domain from any transcription factor of any organism known to the person skilled in the art may be used in the present invention. Preferably, for the transcription factor of the present invention any activation domain of the transcription factor of the present invention of any defined species herein may be used, preferably the activation domain as shown in SEQ ID NO. 83. For the additional transcription factor also any activation domain of the additional transcription factor of any defined species herein may be used. In a further embodiment also a synthetic (such as SEQ ID NO. 84) or a viral (e.g.: VP64) activation domain may also be used in the present invention for the transcription factor of the present invention or for the additional transcription factor. The function of the activation domain can be measured by known methods in the art, i.e. by the yeast-2-Hybrid (Y2H) technique allowing the detection of interacting proteins in living yeast cells. Thus, the transcription factor used in the method, in the recombinant host cell and in the use of the present invention comprises at least a DNA binding domain and an activation domain. The activation domain as shown in SEQ ID NO. 83 or SEQ ID NO.84 may be preferred. It is also contemplated that activation domains from functional homologues may be used. The activation domain specifically for MSN4 of Pichia pastoris may be part of SEQ ID NO. 83.

The present invention further provides a method of increasing the yield of a recombinant protein of interest in a host cell comprising: i) engineering the host cell to overexpress at least one polynucleotide encoding at least one transcription factor of the present invention comprising at least a DNA binding domain and an activation domain, ii) engineering said host cell to comprise a polynucleotide encoding the protein of interest, iii) culturing said host cell under suitable conditions to overexpress the at least one polynucleotide encoding at least one transcription factor and to overexpress the protein of interest, optionally iv) isolating the protein of interest from the cell culture, and optionally v) purifying the protein of interest.

It should be noted that the steps recited in (i) and (ii) does not have to be performed in the recited sequence. It is possible to first perform the step recited in (ii) and then (i). In step (i), the host cell can be engineered to overexpress at least one polynucleotide encoding the at least one transcription factor of the present invention comprising a DNA binding domain comprising an amino acid as shown in SEQ ID NO: 1 or a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87.

When a host cell is “engineered to overexpress” a given protein, the host cell is manipulated such that the host cell has the capability to express, preferably overexpress the transcription factor or functional homologue thereof of the present invention, thereby expression of a given protein, e.g. POI or model protein is increased compared to the host cell under the same condition prior to manipulation. In one embodiment, “engineered to overexpress” implies that a genetic alteration to a host cell is made in order to increase expression of a protein, i.e. the cell is (intentionally) genetically engineered to overexpress such protein.

“Prior to engineering” or “prior to manipulation” when used in the context of host cells of the present invention means that such host cells are not engineered using a polynucleotide encoding the transcription factor or functional homologue thereof of the present invention. Said term thus also means that host cells do not overexpress a polynucleotide encoding the transcription factor or functional homologue thereof of the present invention or are not engineered to overexpress a polynucleotide encoding the transcription factor or functional homologue thereof of the present invention. Thus a “host cell prior to engineering” or a “host cell prior to manipulation” or a “host cell which does not overexpress the polynucleotide encoding the transcription factor” is a host cell not overexpressing a polynucleotide encoding the transcription factor or functional homologue thereof of the present invention or a host cell not engineered to overexpress a polynucleotide encoding the transcription factor or functional homologue thereof of the present invention. Furthermore, the “host cell prior to engineering” or the “host cell prior to manipulation” or the “host cell which does not overexpress the polynucleotide encoding the transcription factor” is the same host cell to which the increase of the yield of said recombinant protein of interest is compared to but without overexpressing a polynucleotide encoding the transcription factor or functional homologue thereof of the present invention or without being engineered to overexpress a polynucleotide encoding the transcription factor or functional homologue thereof of the present invention.

The term “engineering said host cell to comprise a polynucleotide encoding said protein of interest” as used herein means that a host cell of the present invention is equipped with a polynucleotide encoding a protein of interest, i.e., a host cell of the present invention is engineered to contain a polynucleotide encoding a protein of interest. This can be achieved, e.g., by transformation or transfection or any other suitable technique known in the art for the introduction of a polynucleotide into a host cell.

Procedures used to manipulate polynucleotide sequences, e.g. coding for the transcription factor and/or the POI, the promoters, enhancers, leaders, etc., are well known to persons skilled in the art, e.g. described by J. Sambrook et al., Molecular Cloning: A Laboratory Manual (3rd edition), Cold Spring Harbor Laboratory, Cold Spring Harbor Laboratory Press, New York (2001).

A foreign or target polynucleotide such as the polynucleotides encoding the overexpressed transcription factor or POI can be inserted into the chromosome by various means, e.g., by homologous recombination or by using a hybrid recombinase that specifically targets sequences at the integration sites. The foreign or target polynucleotide described above is typically present in a vector (“inserting vector”). These vectors are typically circular and linearized before used for homologous recombination. As an alternative, the foreign or target polynucleotides may be DNA fragments joined by fusion PCR or synthetically constructed DNA fragments which are then recombined into the host cell. In addition to the homology arms, the vectors may also contain markers suitable for selection or screening, an origin of replication, and other elements. It is also possible to use heterologous recombination which results in random or non-targeted integration. Heterologous recombination refers to recombination between DNA molecules with significantly different sequences. Methods of recombinations are known in the art and for example described in Boer et al., Appl Microbiol Biotechnol (2007) 77:513-523. One may also refer to Principles of Gene Manipulation and Genomics by Primrose and Twyman (7^{th} edition, Blackwell Publishing 2006) for genetic manipulation of yeast cells.

Polynucleotides encoding the overexpressed transcription factor and/or POI may also be present on an expression vector. Such vectors are known in the art. In expression vectors, a promoter is placed upstream of the gene encoding the heterologous protein and regulates the expression of the gene. Multi-cloning vectors are especially useful due to their multi-cloning site. For expression, a promoter is generally placed upstream of the multi-cloning site. A vector for integration of the polynucleotide encoding the transcription factor and/or the POI may be constructed either by first preparing a DNA construct containing the entire DNA sequence coding for the transcription factor and/or the POI and subsequently inserting this construct into a suitable expression vector, or by sequentially inserting DNA fragments containing genetic information for the individual elements, such as the DNA binding domain, the activation domain, followed by ligation. As an alternative to restriction and ligation of fragments, recombination methods based on attachment sites (att) and recombination enzymes may be used to insert DNA sequences into a vector. Such methods are described, for example, by Landy (1989) Ann. Rev. Biochem. 58:913-949; and are known to those of skill in the art.

Host cells according to the present invention can be obtained by introducing a vector or plasmid comprising the target polynucleotide sequences into the cells. Techniques for transfecting or transforming eukaryotic cells or transforming prokaryotic cells are well known in the art. These can include lipid vesicle mediated uptake, heat shock mediated uptake, calcium phosphate mediated transfection (calcium phosphate/DNA co-precipitation), viral infection, particularly using modified viruses such as, for example, modified adenoviruses, microinjection and electroporation. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylene glycol mediated uptake. The DNA can be single or double stranded, linear or circular, relaxed or supercoiled DNA. For various techniques for transfecting mammalian cells, see, for example, Keown et al. (1990) Processes in Enzymology 185:527-537.

The phrase “culturing said host cell under suitable conditions to overexpress the at least one polynucleotide encoding at least one transcription factor and to overexpress the protein of interest” refers to maintaining and/or growing eukaryotic host cells under conditions (e.g., temperature, pressure, pH, induction, growth rate, medium, duration, etc.) appropriate or sufficient to obtain production of the desired compound (P01) or to obtain or to overexpress the transcription factor of the present invention.

A host cell according to the invention obtained by transformation with the transcription factor gene(s), and/or the POI gene(s) may preferably first be cultivated at conditions to grow efficiently to a large cell number without the burden of expressing a recombinant protein. When the cells are prepared for POI expression, suitable cultivation conditions are selected and optimized to produce the POI.

By way of example, using different promoters and/or copies and/or integration sites for the transcription factor(s) and the POI(s), the expression of the transcription factor(s) can be controlled with respect to time point and strength of induction in relation to the expression of the POI(s). For example, prior to induction of POI expression, the transcription factor may be first expressed. This has the advantage that the transcription factor is already present at the beginning of POI translation. Alternatively, the transcription factor and POI(s) can be induced at the same time.

An inducible promoter may be used that becomes activated as soon as an inductive stimulus is applied, to direct transcription of the gene under its control. Under growth conditions with an inductive stimulus, the cells usually grow more slowly than under normal conditions, but since the culture has already grown to a high cell number in the previous stage, the culture system as a whole produces a large amount of the recombinant protein. An inductive stimulus is preferably the addition of an appropriate agents (e.g. methanol for the AOX-promoter) or the depletion of an appropriate nutrient (e.g., methionine for the MET3-promoter). Also, the addition of ethanol, methylamine, cadmium or copper as well as heat or an osmotic pressure increasing agent can induce the expression depending on the promotors operably linked to the transcription factor and the POI(s).

It is preferred to cultivate the host cell(s) according to the invention in a bioreactor under optimized growth conditions to obtain a cell density of at least 1 g/L, preferably at least 10 g/L cell dry weight, more preferably at least 50 g/L cell dry weight. It is advantageous to achieve such yields of biomolecule production not only on a laboratory scale, but also on a pilot or industrial scale.

According to the present invention, due to overexpression of the at least one transcription factor, the POI is obtainable in high yields, even when the biomass is kept low. Thus, a high specific yield, which is measured in mg POI/g dry biomass, may be in the range of 1 to 200, such as 50 to 200, such as 100-200, in the laboratory, pilot and industrial scale is feasible. The specific yield of a production host cell according to the invention preferably provides for an increase of at least 1.1 fold, more preferably at least 1.2 fold, at least 1.3 or at least 1.4 fold, in some cases an increase of more than 2 fold can be shown, when compared to the expression of the product without the overexpression of the at least one transcription factor.

The host cell according to the invention may be tested for its expression/secretion capacity or yield by measuring the titer of the protein of interest in the supernatant of the cell culture or the cell homogenate of the cells after cell homogenisation by using standard tests, e.g. ELISA, activity assays, HPLC, Surface Plasmon Resonance (Biacore), Western Blot, capillary electrophoresis (Caliper) or SDS-Page.

Preferably, the host cells are cultivated in a minimal medium with a suitable carbon source, thereby further simplifying the isolation process significantly. By way of example, the minimal medium contains an utilizable carbon source (e.g. glucose, glycerol, ethanol or methanol), salts containing the macro elements (potassium, magnesium, calcium, ammonium, chloride, sulphate, phosphate) and trace elements (copper, iodide, manganese, molybdate, cobalt, zinc, and iron salts, and boric acid).

In the case of yeast cells, the cells may be transformed with one or more of the above-described expression vector(s), mated to form diploid strains, and cultured in conventional nutrient media modified as appropriate for inducing promoters, selecting transformants or amplifying the genes encoding the desired sequences. A number of minimal media suitable for the growth of yeast are known in the art. Any of these media may be supplemented as necessary with salts (such as sodium chloride, calcium, magnesium, and phosphate), buffers (such as HEPES, citric acid and phosphate buffer), nucleosides (such as adenosine and thymidine), antibiotics, trace elements, vitamins, and glucose or an equivalent energy source. Any other necessary supplements may also be included at appropriate concentrations that would be known to those skilled in the art. The culture conditions, such as temperature, pH and the like, are those previously used with the host cell selected for expression and are known to the ordinarily skilled artisan. Cell culture conditions for other type of host cells are also known and can be readily determined by the artisan. Descriptions of culture media for various microorganisms are for example contained in the handbook “Manual of Methods for General Bacteriology” of the American Society for Bacteriology (Washington D.C, USA, 1981).

Host cells can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In some embodiments, cells are cultured in shake flasks or deep well plates. In yet other embodiments, cells are cultured in a bioreactor (e.g., in a bioreactor cultivation process). Cultivation processes include, but are not limited to, batch, fed-batch and continuous methods of cultivation. The terms “batch process” and “batch cultivation” refer to a closed system in which the composition of media, nutrients, supplemental additives and the like is set at the beginning of the cultivation and not subject to alteration during the cultivation; however, attempts may be made to control such factors as pH and oxygen concentration to prevent excess media acidification and/or cell death. The terms “fed-batch process” and “fed-batch cultivation” refer to a batch cultivation with the exception that one or more substrates or supplements are added (e.g., added in increments or continuously) as the cultivation progresses. The terms “continuous process” and “continuous cultivation” refer to a system in which a defined cultivation media is added continuously to a bioreactor and an equal amount of used or “conditioned” media is simultaneously removed, for example, for recovery of the desired product. A variety of such processes has been developed and is well-known in the art.

In some embodiments, host cells are cultured for about 12 to 24 hours, in other embodiments, host cells are cultured for about 24 to 36 hours, about 36 to 48 hours, about 48 to 72 hours, about 72 to 96 hours, about 96 to 120 hours, about 120 to 144 hours, or for a duration greater than 144 hours. In yet other embodiments, culturing is continued for a time sufficient to reach desirable production yields of POI.

The above mentioned methods may further comprise a step of isolating the expressed POI. If the POI is secreted from the cells, it can be isolated and purified from the culture medium using state of the art techniques. Secretion of the POI from the cells is generally preferred, since the products are recovered from the culture supernatant rather than from the complex mixture of proteins that results when cells are disrupted to release intracellular proteins. A protease inhibitor, such as phenyl methyl sulfonyl fluoride (PMSF) may be useful to inhibit proteolytic degradation during purification, and antibiotics may be included to prevent the growth of adventitious contaminants. The composition may be concentrated, filtered, dialyzed, etc., using methods known in the art. The cell culture after fermentation/cultivation can be centrifuged using a separator or a tube centrifuge to separate the cells from the culture supernatant. The supernatant can then be filtered of concentrated by using a tangential flow filtration. Alternatively, cultured host cells may also be ruptured sonically or mechanically (e.g. high pressure homogenisation), enzymatically or chemically to obtain a cell extract containing the desired POI, from which the POI may be isolated and purified.

An isolation and purification methods for obtaining the POI may be based on methods utilizing difference in solubility, such as salting out, solvent precipitation, heat precipitation, methods utilizing difference in molecular weight, such as size exclusion chromatography, ultrafiltration and gel electrophoresis, methods utilizing difference in electric charge, such as ion-exchange chromatography, methods utilizing specific affinity, such as affinity chromatography, methods utilizing difference in hydrophobicity, such as hydrophobic interaction chromatography and reverse phase high performance liquid chromatography, methods utilizing difference in isoelectric point, such as isoelectric focusing may be used and methods utilizing certain amino acids, such as IMAC (immobilized metal ion affinity chromatography. If the POI is expressed as inactive and soluble Inclusion Bodies the solubilized Inclusion Bodies need to be refolded.

The isolated and purified POI can be identified by conventional methods such as Western Blotting or specific assays for POI activity. The structure of the purified POI can be determined by amino acid analysis, amino-terminal peptide sequencing, primary structure analysis for example by mass spectrometry, RP-HPLC, ion exchange-HPLC, ELISA and the like. It is preferred that the POI is obtainable in large amounts and in a high purity level, thus meeting the necessary requirements for being used as an active ingredient in pharmaceutical compositions or as feed or food additive.

The term “isolated” as used herein means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature, e.g. cDNA made from mRNA; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

The present invention further provides a method of manufacturing a recombinant protein of interest by a eukaryotic host cell comprising (i) providing the host cell engineered to overexpress at least one polynucleotide encoding at least one transcription factor, wherein the host cell further comprises a polynucleotide encoding a protein of interest, wherein the transcription factor of the present invention comprises at least a DNA binding domain and an activation domain, (ii) culturing said host cell under suitable conditions to overexpress the at least one polynucleotide encoding at least one transcription factor or functional homologue thereof and to overexpress the protein of interest and optionally (iii) isolating the protein of interest from the cell culture, and optionally (iv) purifying the protein of interest and optionally (v) modifying the protein of interest and optionally (vi) formulating the protein of interest.

Preferably, in step (i), the host cell is engineered to overexpress at least one polynucleotide encoding the at least one transcription factor of the present invention comprising a DNA binding domain comprising an amino acid as shown in SEQ ID NO: 1 or a functional homolog of the amino acid sequence as shown in SEQ ID NO: 1 having at least 60% sequence identity to an amino acid sequence as shown in SEQ ID NO: 1 and/or having at least 60% sequence identity to an amino acid sequence as shown in SEQ ID NO: 87.

In this context, the term “manufacturing a recombinant protein of interest by/in a eukaryotic host cell” as used herein is meant that the recombinant protein of interest may be manufactured by using a eukaryotic host cell for the formation of the recombinant host cell. Thereby, the eukaryotic host cell may produce the recombinant protein of interest inside the cell and maintain the recombinant POI inside the cell (intracellular) or secrete the recombinant POI into the culture medium (extracellular), where the host cell is cultured therein. Thus the POI may be isolated from said culture medium (supernatant of the cell culture) or the cell homogenate of the cells after cell homogenisation.

In this context, the term “modifying the protein of interest” is meant that the POI is chemically modified. There are many methods known in the art to modify proteins. Proteins can be coupled to carbohydrates or lipids. The POI may be PEGylated (the POI chemically coupled to polyethylenglycole) or HESylated (the POI is chemically coupled to hydroxyethyl starch) for half-life extension. The POI may also be coupled with other moieties such as affinity domains for e.g. human serum albumin for half life extension. The POI also may be treated by a protease or under hydrolytic conditions for cleavage to form the active ingredient from a pre-sequence or to cleaff off a tag such as an affinity tag for purification. The POI may also be coupled to other moieties such as toxins, radioactive moieties or any other moiety. The POI may further be treated under conditions to form dimers, trimers and the like.

Additionally, the term “formulating the protein of interest” refers to bringing the POI to conditions, where the POI can be stored for a longer time. Many different methods known in the art are available to stabilize proteins. By exchanging the buffer in which the POI is existent after purification and/or modification, the POI can be brought under conditions, where it is more stable. Different buffer substances and additives, such as sucrose, mild detergents, stabilizer and the like, known in the art can be used. The POI can also be stabilized by lyophilization. For some POIs formulations can be done by formation of complexes of the POI with lipids or lipoproteins, such als polyplexes, and the like. Some protein may be co-formulated with other proteins.

The overexpression of said Msn4p transcription factor(s) (see SEQ ID NOs: 15-27) of the present invention used in the methods, in the recombinant host cell and the use of the present invention may increase the yield of the model proteins scFv (SEQ ID NO. 13) and/or vHH (SEQ ID NO. 14) compared to the host cell prior to engineering. The yield of the model protein(s) mentioned above may be increased by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. As used herein, the term “0%, 10%, 20%, 30%, 40%, 50%, 60%, 70%, 80%, 90%, 100%, 200%, 300%, 400%, 500%, 600% etc.” refers to “1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold etc. The suffix “-fold” refers to multiples. “Onefold” means a whole, “twofold” means twice as much, “threefold” means three times as much. The overexpression of the native transcription factor Msn4p of P. pastoris of the present invention may increase the yield of the model protein, preferably of the scFv (SEQ ID NO. 13) compared to the host cell prior to engineering by at least 10%, such as 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the synthetic transcription factor synMsn4p of the present invention may increase the yield of the model protein, preferably of the vHH (SEQ ID NO. 14) compared to the host cell prior to engineering by at least 10%, such as 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%.

The polynucleotide encoding the transcription factor(s) and/or the polynucleotide encoding the POI used in the methods, in the recombinant host cell and the use of the present invention is/are preferably integrated into the genome of the host cell. The term “genome” generally refers to the whole hereditary information of an organism that is encoded in the DNA (or RNA for certain viral species). It may be present in the chromosome, on a plasmid or vector, or both. Preferably, the polynucleotide encoding the transcription factor is integrated into the chromosome of said cell.

Polynucleotides encoding the transcription factor(s) and the POI(s) may be recombined in the host cell by ligating the relevant genes each into one vector. It is possible to construct single vectors carrying the genes, or two separate vectors, one to carry the transcription factor genes and the other one the POI genes. These genes can be integrated into the host cell genome by transforming the host cell using such vector or vectors. In some embodiments, the gene encoding the POI is integrated in the genome and the gene encoding the transcription factor is integrated in a plasmid or vector. In some embodiments, the gene(s) encoding the transcription factor is/are integrated in the genome and the gene(s) encoding the POI is/are integrated in a plasmid or vector. In some embodiments, the genes encoding the POI and the transcription factor are integrated in the genome. In some embodiments, the genes encoding the POI and the transcription factor are integrated in a plasmid or vector. If multiple genes encoding the POI are used, some genes encoding the POI can be integrated in the genome while others can be integrated in the same or different plasmids or vectors. If multiple genes encoding the transcription factor(s) are used, some of the genes encoding the transcription factor can be integrated in the genome while others can be integrated in the same or different plasmids or vectors.

The polynucleotide encoding the transcription factor or functional homologue thereof may be integrated in its natural locus. “Natural locus” means the location on a specific chromosome, where the polynucleotide encoding the transcription factor is located, for example at the natural locus of the gene encoding a transcription factor of the present invention. However, in another embodiment, the polynucleotide encoding the transcription factor is present in the genome of the host cell not at their natural locus, but integrated ectopically. The term “ectopic integration” means the insertion of a nucleic acid into the genome of a microorganism at a site other than its usual chromosomal locus, i.e., predetermined or random integration. In the alternative, the polynucleotide encoding the transcription factor or functional homologue thereof may be integrated in its natural locus and ectopically.

For yeast cells, the polynucleotide encoding the transcription factor and/or the polynucleotide encoding the POI may be inserted into a desired locus, such as but not limited to AOX1, GAP, ENO1, TEF, HIS4 (Zamir et al., Proc. NatL Acad. Sci. USA (1981) 78(6):3496-3500), HO (Voth et al. Nucleic Acids Res. 2001 Jun. 15; 29(12): e59), TYR1 (Mirisola et al., Yeast 2007; 24: 761-766), His3, Leu2, Ura3 (Taxis et al., BioTechniques (2006) 40:73-78), Lys2, ADE2, TRP1, GAL1, ADH1, RGI1 or in the ribosomal RNA gene locus.

In other embodiments, the polynucleotide encoding the at least one transcription factor and/or the polynucleotide encoding the POI can be integrated in a plasmid or vector. The terms “plasmid” and “vector” include autonomously replicating nucleotide sequences as well as genome integrating nucleotide sequences. A skilled person is able to employ suitable plasmids or vectors depending on the host cell used.

Preferably, the plasmid is a eukaryotic expression vector, preferably a yeast expression vector.

Plasmids can be used for the transcription of cloned recombinant nucleotide sequences, i.e. of recombinant genes and the translation of their mRNA in a suitable host organism. Plasmids can also be used to integrate a target polynucleotide into the host cell genome by methods known in the art, such as described by J. Sambrook et al., Molecular Cloning: A Laboratory Manual (3rd edition), Cold Spring Harbor Laboratory, Cold Spring Harbor Laboratory Press, New York (2001). A “plasmid” usually comprise an origin for autonomous replication, selectable markers, a number of restriction enzyme cleavage sites, a suitable promoter sequence and a transcription terminator, which components are operably linked together. The polypeptide coding sequence of interest is operably linked to transcriptional and translational regulatory sequences that provide for expression of the polypeptide in the host cells.

A nucleic acid is “operably linked” when it is placed into a functional relationship with another nucleic acid sequence on the same nucleic acid molecule. For example, a promoter is operably linked with a coding sequence of a recombinant gene when it is capable of effecting the expression of that coding sequence.

Most plasmids exist in only one copy per bacterial cell. Some plasmids, however, exist in higher copy numbers. For example, the plasmid ColE1 typically exists in 10 to 20 plasmid copies per chromosome in E. coli. If the nucleotide sequences of the present invention are contained in a plasmid, the plasmid may have a copy number of 1-10, 10-20, 20-30, 30-100 or more per host cell. With a high copy number of plasmids, it is possible to overexpress transcription factor by the cell.

Large numbers of suitable plasmids or vectors are known to those of skill in the art and many are commercially available. Examples of suitable vectors are provided in Sambrook et al, eds., Molecular Cloning: A Laboratory Manual (2nd Ed.), Vols. 1-3, Cold Spring Harbor Laboratory (1989), and Ausubel et al, eds., Current Protocols in Molecular Biology, John Wiley & Sons, Inc., New York (1997).

A vector or plasmid of the present invention encompass yeast artificial chromosome, which refers to a DNA construct that can be genetically modified to contain a heterologous DNA sequence (e.g., a DNA sequence as large as 3000 kb), that contains telomeric, centromeric, and origin of replication (replication origin) sequences.

A vector or plasmid of the present invention also encompasses bacterial artificial chromosome (BAC), which refers to a DNA construct that can be genetically modified to contain a heterologous DNA sequence (e.g., a DNA sequence as large as 300 kb), that contains an origin of replication sequence (Ori), and may contain one or more helicases (e.g., parA, parB, and parC).

Examples of plasmids using yeast as a host include YIp type vector, YEp type vector, YRp type vector, YCp type vector (Yxp vectors are e.g. described in Romanos et al. 1992, Yeast. 8(6):423-488), pGPD-2 (described in Bitter et al., 1984, Gene, 32:263-274), pYES, pAO815, pGAPZ, pGAPZa, pHIL-D2, pHIL-S1, pPIC3.5K, pPIC9K, pPICZ, pPICZa, pPIC3K, pPINK-HC, pPINK-LC (all available from Thermo Fisher Scientific/Invitrogen), pHWO10 (described in Waterham et al., 1997, Gene, 186:37-44), pPZeoR, pPKanR, pPUZZLE and pPUZZLE-derivatives such as pPM2d, pPM2aK21 or pPM2eH21 (described in Stadlmayr et al., 2010, J Biotechnol. 150(4):519-29; Marx et al. 2009, FEMS Yeast Res. 9(8):1260-70.); GoldenPiCS system (consisting of the backbones BB1, BB2 and BB3aK/BB3eH/BB3rN); pJ-vectors (e.g. pJAN, pJAG, pJAZ and their derivatives; all available from BioGrammatics, Inc), pJexpress-vectors, pD902, pD905, pD915, pD912 and their derivatives, pD12xx, pJ12xx (all available from ATUM/DNA2.0), pRG plasmids (described in Gnügge et al., 2016, Yeast 33:83-98) 2 μm plasmids (described e.g. in Ludwig et al., 1993, Gene 132(1):33-40). Such vectors are known and are for example described in Cregg et al., 2000, Mol Biotechnol. 16(1):23-52 or Ahmad et al. 2014, Appl Microbiol Biotechnol. 98(12):5301-17. Additionally suitable vectors can be readily generated by advanced modular cloning techniques as for example described by Lee et al. 2015, ACS Synth Biol. 4(9):975-986; Agmon et al. 2015, ACS Synth. Biol., 4(7):853-859; or Wagner and Alper, 2016, Fungal Genet Biol. 89:126-136. Additionally, these and other suitable vectors may be also available from Addgene, Cambridge, Mass., USA.

Preferably, a BB1 plasmid of the GoldenPiCS system is used to introduce the gene fragments of the transcription factor of the present invention by using specific restriction enzymes (Table 1). The assembled BB1s carrying the respective coding sequence may then further be processed in the GoldenPiCS system to create the required BB3 integration plasmids as described in Prielhofer et al. 2017.

The polynucleotide encoding at least one transcription factor used in the methods, in the recombinant host cell and the use of the present invention may encode for a heterologous or homologous transcription factor.

As used herein, the term “heterologous” means derived from a cell or organism (preferably yeast) with a different genomic background or a synthetic sequence. Thus, a “heterologous transcription factor” is one that originates from a foreign source (or species, e.g. Msn4p of S. cerevisiae or synMsn4p) and is being used in the source (or species e.g. P. pastoris) other than the foreign source. The term “homologous” means derived from the same cell or organismus with the same genomic background. Thus, a “homologous transcription factor” is one that originates from the same source (or species, e.g. Msn4p of P. pastoris) and is being used in the same source (or species e.g. P. pastoris).

In general, overexpression can be achieved in any ways known to a skilled person in the art as will be described later in detail. It can be achieved by increasing transcription/translation of the gene, e.g. by increasing the copy number of the gene or altering or modifying regulatory sequences. For example, overexpression can be achieved by introducing one or more copies of the polynucleotide encoding the transcription factor or a functional homologue operably linked to regulatory sequences (e.g. a promoter). For example, the gene can be operably linked to a strong constitutive promoter in order to reach high expression levels. Such promoters can be endogenous promoters or recombinant promoters. Alternatively, it is possible to remove regulatory sequences such that expression becomes constitutive. One can substitute the native promoter of a given gene with a heterologous promoter which increases expression of the gene or leads to constitutive expression of the gene. For example, the transcription factor may be overexpressed by more than 10%, 20%, 30%, 40%, 50%, 60%, 70%, 80%, 90%, 100%, 200%, or more than 300% by the host cell compared to the host cell prior to engineering and cultured under the same conditions. Furthermore, overexpression can also be achieved by, for example, modifying the chromosomal location of a particular gene, altering nucleic acid sequences adjacent to a particular gene such as a ribosome binding site or transcription terminator, modifying proteins (e.g., regulatory proteins, suppressors, enhancers, transcriptional activators and the like) involved in transcription of the gene and/or translation of the gene product, or any other conventional means of deregulating expression of a particular gene routine in the art including but not limited to use of antisense nucleic acid molecules, for example, to block expression of repressor proteins or deleting or mutating the gene for a transcriptional factor which normally represses expression of the gene desired to be overexpressed. Prolonging the life of the mRNA may also improve the level of expression. For example, certain terminator regions may be used to extend the half-lives of mRNA (Yamanishi et al., Biosci. Biotechnol. Biochem. (2011) 75:2234 and US 2013/0244243). If multiple copies of genes are included, the genes can either be located in plasmids of variable copy number or integrated and amplified in the chromosome. If the host cell does not comprise the gene encoding the transcription factor, it is possible to introduce the gene into the host cell for expression. In this case, “overexpression” means expressing the gene product using any methods known to a skilled person in the art.

Those skilled in the art will find relevant instructions in Martin et al. (Bio/Technology 5, 137-146 (1987)), Guerrero et al. (Gene 138, 35-41 (1994)), Tsuchiya and Morinaga (Bio/Technology 6, 428-430 (1988)), Eikmanns et al. (Gene 102, 93-98 (1991)), EP 0 472 869, U.S. Pat. No. 4,601,893, Schwarzer and Pühler (Bio/Technology 9, 84-87 (1991)), Reinscheid et al. (Applied and Environmental Microbiology 60, 126-132 (1994)), LaBarre et al. (Journal of Bacteriology 175, 1001-1007 (1993)), WO 96/15246, Malumbres et al. (Gene 134, 15-24 (1993)), JP-A-10-229891, Jensen and Hammer (Biotechnology and Bioengineering 58, 191-195 (1998)) and Makrides (Microbiological Reviews 60, 512-538 (1996)), inter alia, and in well-known textbooks on genetics and molecular biology.

Thus, the overexpression of the polynucleotide encoding a heterologous transcription factor used in the methods, in the recombinant host cell and the use of the present invention may be achieved by exchanging or modifying a regulatory sequence operably linked to said polynucleotide encoding the heterologous transcription factor. In this context, a “regulatory sequence (element)” is a segment of a nucleic acid molecule which is capable of increasing or decreasing the expression of specific genes within an organism. A positive regulatory sequence is capable of increasing the expression, whereas a negative regulatory sequence is capable of decreasing the expression. A regulatory sequence (element) includes for example, promoters, enhancers, silencers, polyadenylation signals, transcription terminators (terminator sequence), coding sequences, internal ribosome entry sites (IRES), and the like. A positive regulatory sequence may comprise, but is not limited to, an enhancer. A negative regulatory sequence may comprise, but is not limited to, a silencer. By exchanging a regulatory sequence in this context, it is meant exchanging the native terminator sequence of said heterologous transcription factor by a more efficient terminator sequence, or exchanging the coding sequence of said heterologous transcription factor by a codon-optimized coding sequence, which codon-optimization is done according to the codon-usage of said host cell, or exchanging of a native positive regulatory element of said heterologous transcription factor by a more efficient regulatory element.

The overexpression of the polynucleotide encoding a heterologous transcription factor used in the methods, in the recombinant host cell and the use of the present invention may further be achieved by introducing one or more copies of the polynucleotide encoding the heterologous transcription factor under the control of a promoter into the host cell.

The term “promoter” as used herein refers to a region that facilitates the transcription of a particular gene. A promoter typically increases the amount of recombinant product expressed from a nucleotide sequence as compared to the amount of the expressed recombinant product when no promoter exists. A promoter from one organism can be utilized to enhance recombinant product expression from a sequence that originates from another organism. The promoter can be integrated into a host cell chromosome by homologous recombination using methods known in the art (e.g. Datsenko et al, Proc. Natl. Acad. Sci. U.S.A., 97(12): 6640-6645 (2000)). In addition, one promoter element can increase the amount of products expressed for multiple sequences attached in tandem. Hence, one promoter element can enhance the expression of one or more recombinant product. Promoter activity may be assessed by its transcriptional efficiency. This may be determined directly by measurement of the amount of mRNA transcription from the promoter, e.g. by Northern Blotting, quantitative PCR or indirectly by measurement of the amount of gene product expressed from the promoter.

The promoter could be an “inducible promoter” or “constitutive promoter.” “Inducible promoter” refers to a promoter which can be induced by the presence or absence of certain factors, and “constitutive promoter” refers to a promoter that is active all the time, independent of an inducer, and therefore allows for continuous transcription of its associated gene or genes.

In a preferred embodiment, both the transcription of the nucleotide sequences encoding the transcription factor and the POI are each driven by an inducible promoter. In another preferred embodiment, both the transcription of the nucleotide sequences encoding the transcription factor and the POI are each driven by a constitutive promoter. In yet another preferred embodiment, the transcription of the nucleotide sequence encoding the transcription factor is driven by a constitutive promoter and the transcription of the nucleotide sequence encoding the POI is driven by an inducible promoter. In yet another preferred embodiment, the transcription of the nucleotide sequences encoding the transcription factor is driven by an inducible promoter and the transcription of the nucleotide sequence encoding the POI is driven by a constitutive promoter. As an example, the transcription of the nucleotide sequence encoding the transcription factor may be driven by a constitutive GAP promoter and the transcription of the nucleotide sequence encoding the POI may be driven by an inducible AOX promoter. In one embodiment, the transcription of the nucleotide sequences encoding the transcription factor and the POI is driven by the same promoter or similar promoters in terms of promoter activity, promoter regulation and/or expression behaviour. In another embodiment, the transcription of the nucleotide sequences encoding the transcription factor and the POI are driven by different promoters in terms of promoter activity, promoter regulation and/or expression behaviour.

Suitable promoter sequences for use with yeast host cells are described in Mattanovich et al., Methods Mol. Biol. (2012) 824:329-58 and include the promoters of glycolytic enzymes like triosephosphate isomerase (TPI), 3-phosphoglycerate kinase (PGK), glucose-6-phosphate isomerase (PGI), glyceraldehyde-3-phosphate dehydrogenase (GAPDH or GAP) and variants thereof, promoters of lactase (LAC) and galactosidase (GAL), translation elongation factor promoter (PTEF), and the promoters of P. pastoris enolase 1 (ENO1), triose phosphate isomerase (TPI), ribosomal subunit proteins (RPS2, RPS7, RPS31, RPL1), alcohol oxidase promoter (AOX) or variants thereof with modified characteristics, the formaldehyde dehydrogenase promoter (FLD), isocitrate lyase promoter (ICL), alpha-ketoisocaproate decarboxylase promoter (THI), the promoters of heat shock protein family members (SSA1, HSP90, KAR2), 6-Phosphogluconate dehydrogenase (GND1), phosphoglycerate mutase (GPM1), transketolase (TKL1), phosphatidylinositol synthase (PIS1), ferro-02-oxidoreductase (FET3), high affinity iron permease (FTR1), repressible alkaline phosphatase (PHO8), N-myristoyl transferase (NMT1), pheromone response transcription factor (MCM1), ubiquitin (UBI4), single-stranded DNA endonuclease (RAD2), the promoter of the major ADP/ATP carrier of the mitochondrial inner membrane (PET9) (WO2008/128701) and the formate dehydrogenase (FDH) promoter. Further suitable promoters are described by Prielhofer et al. 2017 (BMC Syst Biol. 11(1):123.), Gasser et al. 2015 (Microb Cell Fact. 14:196.), Portela et al. 2017. (ACS Synth Biol. 6(3):471-484.) or Vogl et al. 2016 (ACS Synth Biol. 5(2):172-86.) AOX promoters can be induced by methanol and are repressed by e.g. glucose.

Further examples of suitable promoters include the promoters of Saccharomyces cerevisiae enolase (ENO-1), galactokinase (GAL1), alcohol dehydrogenase/glyceraldehyde-3-phosphate dehydrogenase (ADH1, ADH2/GAP), triose phosphate isomerase (TPI), metallothionein (CUP1), 3-phosphoglycerate kinase (PGK), and the maltase gene promoter (MAL).

Other useful promoters for yeast host cells are described by Romanos et al, 1992, Yeast 8:423-488.

Each coding sequence of the heterologous transcription factor (e.g. synMsn4p) of the present invention may be combined with the GAP promoter into a integration plasmid, preferably BB3.

The overexpression of the polynucleotide encoding a homologous transcription factor used in the methods, in the recombinant host cell and the use of the present invention may be achieved by using a promoter which drives expression of said polynucleotide encoding the homologous transcription factor. The endogenous/native promoter operably linked to the endogenous, homologous transcription factor may be replaced with another stronger promoter in order to reach high expression levels. Such promoter may be inducible or constitutive. Modification and/or replacement of the endogenous promoter may be performed by mutation or homologous recombination using methods known in the art.

Each coding sequence of the homologous transcription factor (e.g. native Msn4p of P. pastoris if expressed in P. pastoris) of the present invention may be combined with a strong constitutive or inducible promoter such as GAP promoter, pTHI11, pSBH17 or pPOR1 or the like into a integration plasmid, such as BB3.

The overexpression of the polynucleotide encoding the transcription factor, can be achieved by other methods known in the art, for example by genetically modifying their endogenous regulatory regions, as described by Marx et al., 2008 (Marx, H., Mattanovich, D. and Sauer, M. Microb Cell Fact 7 (2008): 23), and Pan et al., 2011 (Pan et al., FEMS Yeast Res. (2011) May; (3):292-8.), such methods include, for example, integration of a recombinant promoter that increases expression of the transcription factor(s). Transformation is described in Cregg et al. (1985) Mol. Cell. Biol. 5:3376-3385.

Thus, the present invention may comprise the overexpression of the polynucleotide encoding a homologous transcription factor used in the methods, in the recombinant host cell and the use of the present invention, being further achieved by exchanging or modifying a regulatory sequence operably linked to said polynucleotide encoding the homologous transcription factor.

By exchanging a regulatory sequence in this context, it is meant for example exchanging the native terminator sequence of said homologous transcription factor by a more efficient terminator sequence, or exchanging the coding sequence of said homologous transcription factor by a codon-optimized coding sequence, which codon-optimization is done according to the codon-usage of said host cell, or exchanging of a native positive regulatory element of said homologous transcription factor by a more efficient positive regulatory element.

As used herein in this context, the term “modifying a regulatory sequence” means addition of another positive regulatory sequence or deletion of a negative regulatory sequence. Thus, modifying a regulatory sequence refers to introducing/adding another positive regulatory sequence, which is not present in the native expression cassette of said homologous/heterologous transcription factor (element) or deleting a negative regulatory sequence (element) which is normally present in the native expression cassette of said homologous/heterologous transcription factor. Native expression cassette means the sequence coding for a protein including its 5′ and 3′ flanking sequences involved in negative or positive regulation of the expression of said protein, such as promoters, terminators, polyadenylation signals, etc. which is present in a cell in nature and which was not artificially generated by man using recombinant gene technology. There may be heterologous as well as homologous native expression cassettes. If an expression cassette from one species is transferred to another species and still results in expression of the protein coded by said native expression cassette, this native expression cassette is then regarded as a heterologous native expression cassette.

The overexpression of the polynucleotide encoding a homologous transcription factor used in the methods, in the recombinant host cell and the use of the present invention may be further achieved by introducing one or more copies of the polynucleotide encoding the homologous transcription factor under the control of a promoter into the host cell.

The overexpression of the polynucleotide encoding at least one transcription factor used in the methods, in the recombinant host cell and the use of the present invention is achieved by i) exchanging the native promoter of said homologous transcription factor by a different promoter, such as a stronger promoter, operably linked to the polynucleotide encoding the homologous transcription factor, ii) exchanging the native terminator sequence of said heterologous and/or homologous transcription factor by a more efficient terminator sequence, iii) exchanging the coding sequence of said heterologous and/or homologous transcription factor by a codon-optimized coding sequence (such as optimized for mRNA stability or half life or for using the most frequent codons and the like), which codon-optimization is done according to the codon-usage of said host cell, iv) exchanging a native positive regulatory element of said heterologous and/or homologous transcription factor by a more efficient regulatory element, v) introducing another positive regulatory element, which is not present in the native expression cassette of said homologous transcription factor, vi) deleting a negative regulatory element, which is normally present in the native expression cassette of said homologous transcription factor, or vii) introducing one or more copies of the polynucleotide encoding a heterologous and/or homologous transcription factor, or a combination thereof.

The present invention may further comprise transcription factor(s) used in the methods, in the recombinant host cell and the use of the present invention comprising an amino acid sequence as shown in SEQ ID NOs: 15-27 or a functional homolog of the amino acid sequence as shown in SEQ ID NO.: 15 having at least 11% sequence identity to the amino acid sequence as shown in SEQ ID NO: 15. In a further embodiment the present invention may further comprise transcription factor(s) used in the methods, in the recombinant host cell and the use of the present invention comprising an amino acid sequence as shown in SEQ ID NOs: 15-27 or a functional homolog of the amino acid sequence as shown in SEQ ID NO.: 15 having at least 11%, such as 15%, 20%, 25%, 30%, 35%, 40%, 45%, 50%, 55%, 60%, 65%, 70%, 75%, 80%, 85%, 90%, 95%, 98% or even 100% sequence identity to the amino acid sequence as shown in SEQ ID NO: 15.

The transcription factor(s) used in the methods, in the recombinant host cell and the use of the present invention may additionally comprise any nuclear localization signal (NLS). Thus, the transcription factor of the present invention may comprise an DNA binding domain as described elsewhere herein, any activation domain as described elsewhere herein and any NLS. Any NLS in this specific context may comprise a synthetic NLS (such as SEQ ID NO. 86) or a viral NLS or an NLS of the transcription factor of the present invention or other proteins of any species as described herein. A NLS is an amino acid sequence that ‘tags’ a protein for import into the cell nucleus by nuclear transport. Typically, a NLS consists of one or more short sequences of positively charged lysines or arginines exposed on the protein surface. The amino acid sequence as shown in SEQ ID NO. 85 (predicted NLS of Msn4p of P. pastoris: EPRKKETKQRKRAK; according to best prediction (score>0.89) by SeqNLS; http://mleg.cse.sc.edu/seqNLS/MainProcess.cgi) or SEQ ID NO. 86 (NLS of synMsn4p: PKKKRKV) is preferred as a NLS in the present invention.

The nuclear localization signal may be a homologous or a heterologous NLS. In this context, the term “heterologous NLS” refers to a NLS that originates from a foreign source (or species, e.g. NLS from S. cerevisiae or human NLS, see also Weninger et al. 2015. FEMS Yeast Res. 15:7) or is a synthetic sequence and is being used in the source (or species e.g. P. pastoris) other than the foreign source. A “homologous NLS” is one that originates from the same source (or species, e.g. NLS of P. pastoris) and is being used in the same source (or species e.g. P. pastoris).

The present invention may further comprise transcription factor(s) used in the methods, in the recombinant host cell and the use of the present invention, wherein said transcription factor(s) does not stimulate the promoter used for expression of the protein of interest. Thereby is meant that the transcription factor of the present invention has no effect on the promoter of the POI. It rather has an effect on the promoter of different proteins other than the POI. In this context, the term “does not stimulate” or “no stimulation” means not having any effect on the promoter of the POI at all or having a light effect on the promoter of the POI, thus resulting in a slight increase of the yield of the POI of about 10% or less, such as an increase of the yield of said POI of 1%, 2%, 3%, 4%, 5%, 6%, 7%, 8%, 9%, or 10%.

The methods, the recombinant host cell and the use of the present invention use a eukaryotic cell as a host cell. As used herein, a “host cell” refers to a cell which is capable of protein expression and optionally protein secretion. Such host cell is applied in the methods of the present invention. For that purpose, for the host cell to overexpress at least one polynucleotide encoding at least one transcription factor, a polynucleotide sequence encoding said transcription factor is present or introduced in the cell. Examples of eukaryotic cells include, but are not limited to, vertebrate cells, mammalian cells, human cells, animal cells, invertebrate cells, plant cells, nematodal cells, insect cells, stem cells, fungal cells or yeast cells.

Preferably, the eukaryotic host cell is a fungal cell. More preferred is a yeast host cell. Examples of yeast cells include but are not limited to the Saccharomyces genus (e.g. Saccharomyces cerevisiae, Saccharomyces kluyveri, Saccharomyces uvarum), the Komagataella genus (Komagataella pastoris, Komagataella pseudopastoris or Komagataella phaffii), Kluyveromyces genus (e.g. Kluyveromyces lactis, Kluyveromyces marxianus), the Candida genus (e.g. Candida utilis, Candida cacaos), the Geotrichum genus (e.g. Geotrichum fermentans), as well as Hansenula polymorpha and Yarrowia lipolytica.

In a preferred embodiment, the genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and renamed to Komagataella pastoris, Komagataella phaffii and Komagataella pseudopastoris. Therefore Pichia pastoris is a synonymous for both Komagataella pastoris, Komagataella phaffii and Komagataella pseudopastoris.

Examples for Pichia pastoris strains useful in the present invention are X33 and its subtypes GS115, KM71, KM71H; CBS7435 (mut+) and its subtypes CBS7435 mut^{s}, CBS7435 mut^{s}4 Δrg, CBS7435 mut^{s}ΔHis, CBS7435 mut^{s}ΔArgΔHis, CBS7435 mut^{s} PDI⁺, CBS704 (=NRRL Y-1603=DSMZ 70382), CBS2612 (=NRRL Y-7556), CBS9173-9189 and DSMZ 70877 as well as mutants thereof. These yeast strains are available from industrial suppliers or cell repositories such as the American Tissue Culture Collection (ATCC), the “Deutsche Sammlung von Mikroorganismen und Zellkulturen” (DSMZ) in Braunschweig, Germany, or from the Dutch “Centraalbureau voor Schimmelcultures” (CBS) in Uetrecht, The Netherlands.

According to a further preferred embodiment, the yeast host cell is selected from the group consisting of Pichia pastoris (Komagataella spp), Hansenula polymorpha, Trichoderma reesei, Aspergillus niger, Saccharomyces cerevisiae, Kluyveromyces lactis, Yarrowia lipolytica, Pichia methanofica, Candida boidinii, Komagataella spp, and Schizosaccharomyces pombe. These yeast strains are available from cell repositories such as the American Tissue Culture Collection (ATCC), the “Deutsche Sammlung von Mikroorganismen und Zellkulturen” (DSMZ) in Braunschweig, Germany, or from the Dutch “Centraalbureau voor Schimmelcultures” (CBS) in Uetrecht, The Netherlands.

The present invention further comprises that the recombinant protein of interest used in the methods, in the recombinant host cell and the use of the present invention may be an enzyme. Preferred enzymes are those which can be used for industrial application, such as in the manufacturing of a detergent, starch, fuel, textile, pulp and paper, oil, personal care products, or such as for baking, organic synthesis, and the like. (see Kirk et al., Current Opinion in Biotechnology (2002) 13:345-351).

The present invention further comprises that the recombinant protein of interest may be a therapeutic protein. A POI may be but is not limited to a protein suitable as a biopharmaceutical substance like an antigen binding protein such as for example an antibody or antibody fragment, or antibody derived scaffold, single domain antibodies and derivatives thereof, other not antibody derived affinity scaffolds such as antibody mimetics, growth factor, hormone, vaccine, etc. as described in more detail herein.

Such therapeutic proteins include, but are not limited to, insulin, insulin-like growth factor, hGH, tPA, cytokines, e.g. interleukines such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosisfactor (TNF) TNF alpha and TNF beta, TRAIL; G-CSF, GM-CSF, M-CSF, MCP-1 and VEGF.

Further examples of therapeutic proteins include blood coagulation factors (VII, VIII, IX), alkaline protease from Fusarium, calcitonin, CD4 receptor darbepoetin, DNase (cystic fibrosis), erythropoetin, eutropin (human growth hormone derivative), follicle stimulating hormone (follitropin), gelatin, glucagon, glucocerebrosidase (Gaucher disease), glucosamylase from A. niger, glucose oxidase from A. niger, gonadotropin, growth factors (GCSF, GMCSF), growth hormones (somatotropines), hepatitis B vaccine, hirudin, human antibody fragment, human apolipoprotein AI, human calcitonin precursor, human collagenase IV, human epidermal growth factor, human insulin-like growth factor, human interleukin 6, human laminin, human proapolipoprotein AI, human serum albumin, insulin, insulin and muteins, insulin, interferon alpha and muteins, interferon beta, interferon gamma (mutein), interleukin 2, luteinization hormone, monoclonal antibody 5T4, mouse collagen, OP-1 (osteogenic, neuroprotective factor), oprelvekin (interleukin 11-agonist), organophosphohydrolase, PDGF-agonist, phytase, platelet derived growth factor (PDGF), recombinant plasminogen-activator G, staphylokinase, stem cell factor, tetanus toxin fragment C, tissue plasminogen-activator, and tumor necrosis factor (see Schmidt, Appl Microbiol Biotechnol (2004) 65:363-372).

Preferably, the therapeutic protein is an antigen binding protein. More preferably, the therapeutic protein comprises an antibody, an antibody fragment or an antibody mimetic. Even more preferably, the therapeutic protein is an antibody or an antibody fragment.

In a preferred embodiment, the protein is an antibody fragment. The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, IgY, etc., from all sources, e.g. human, rodent, rabbit, cow, sheep, pig, dog, other mammals, chicken, other avians, etc., are considered to be “antibodies.” For example, an antibody fragment may include but not limited to Fv (a molecule comprising the VL and VH), single-chain Fv (scFV) (a molecule comprising the VL and VH connected with by peptide linker), Fab, Fab′, F(ab′)₂, single domain antibody (sdAb) (molecules comprising a single variable domain and 3 CDR), and multivalent presentations thereof. The antibody or fragments thereof may be murine, human, humanized or chimeric antibody or fragments thereof. Examples of therapeutic proteins include an antibody, polyclonal antibody, monoclonal antibody, recombinant antibody, antibody fragments, such as Fab′, F(ab′)2, Fv, scFv, di-scFvs, bi-scFvs, tandem scFvs, bispecific tandem scFvs, sdAb, nanobodies, V_{H}, and V_{L}, or human antibody, humanized antibody, chimeric antibody, IgA antibody, IgD antibody, IgE antibody, IgG antibody, IgM antibody, intrabody, diabody, tetrabody, minibody or monobody. Preferably, the antibody fragment is a scFv (SEQ ID NO. 13) and/or vHH (SEQ ID NO. 14). An antibody mimetic refers to an organic compound that binds antigens, but that are not structurally related to antibodies. Such an antibody mimetic refers to artificial peptides or proteins having a molar mass of about 3 to 20 kDA, such as affibody molecules, affilins, affimers, affitins, alphabodies, anticalins, avimers, DARPins, monobodies, nanoCLAMPs as known in the prior art.

The protein of interest may further be a food additive. A food additive is a protein used as nutritional, dietary, digestive, supplements, such as in food products, feed products, or cosmetic products. The food products may be, for example, bouillon, desserts, cereal bars, confectionery, sports drinks, dietary products or other nutrition products. A “food” means any natural or artificial diet meal or the like or components of such meals intended or suitable for being eaten, taken in, digested, by a human being.

The protein of interest may further be a feed additive. Examples of enzymes which can be used as feed additive include phytase, xylanase and β-glucanase.

The methods, the recombinant host cell and the use of the present invention may comprise further overexpressing in said host cell or engineering said host cell to overexpress at least one polynucleotide encoding at least one ER helper protein. In this context, the term “ER” refers to “endoplasmatic reticulum”. Preferably, by further overexpressing in said host cell at least one polynucleotide encoding at least one ER helper protein, the yield of the recombinant protein of interest increases in comparison to a host cell overexpressing at least one polynucleotide encoding at least one transcription factor but not overexpressing at least one polynucleotide encoding at least one ER helper protein.

As used herein, the term “at least one polynucleotide encoding at least one ER helper protein” means one polynucleotide encoding one ER helper protein, two polynucleotides encoding at least two ER helper proteins, three polynucleotides encoding three ER helper proteins etc.

The term “ER helper protein” refers to a chaperone, a co-chaperone and/or a nucleotide exchange factor. The term “chaperone” as used herein relates to a polypeptide that assist the folding, unfolding, assembly or disassembly of other polypeptides. A chaperone refers to proteins that are involved in the correct folding or unfolding and transportation of newly translated eukaryotic cytosolic and secretory proteins. There are many different families of chaperones, each family acts to aid protein folding in a different way. There are ER chaperones and cytosolic chaperones.

Cytosolic chaperones in yeast cells comprise but are not limited to Ssa1p, Ssa2p, Ssa3p, Ssa4p, Ssb1p, Ssb2p, Sse1p, Sse2p, which refer to the Hsp70 system. Ssa1-4p are involved in the folding of newly synthesized proteins, and transportation of intermediate proteins to the ER and mitochondria. Ssb1p and Ssb2p are involved in folding of ribosome-bound nascent chains and Sse1p and Sse2p act as nucleotide exchange factors for Ssap and Ssbp. Ydj1p and Sis1p belong to the Hsp40 system in yeast and interact as co-chaperones with non-native polypeptides triggering ATP hydrolysis by Ssa1-4p and are involved in protein transport across membranes. Snl1p, Fes1p, Cns1p are other co-chaperones of Ssa1-4p (Chang et al., Cell 128 (2007)). In this context, the term “co-chaperone” refers to a protein that assists a chaperone in protein folding and other functions. A co-chaperone is the non-client binding molecules that assists in protein folding mediated by Hsp70 and Hsp90.

ER chaperones in yeast cells comprise but are not limited to Kar2p for example, which refers to the Hsp70 system or Pdi1p. Kar2p is involved in protein translocation into ER, binding to unassembled/misfolded ER protein subunits and regulating unfolded protein response (UPR). It interacts with its co-chaperones such as Lhs1p, Sil1p, Erj5p, Sec63p, Scj1p, Jem1p or others known in the art. Lhs1p and Sil1p refer to nucleotide exchange factors of Kar2p and belong to the Hsp70 system (Chang et al., Cell 128 (2007)). In this context, the term “nucleotide exchange factor” refers to a protein that stimulates the exchange (replacement) of nucleoside diphosphates (ADP, GDP) for nucleoside triphosphates (ATP, GTP) bound to other proteins (preferably to chaperones). Erj5p, Sec63 and Scj1 belong to the group of Hsp40 type proteins. Erj5p for example is a type I membrane protein with a J domain; required to preserve the folding capacity of the endoplasmic reticulum; loss of the non-essential ERJ5 gene leads to a constitutively induced unfolded protein response (Mehnert et al., Molecular biology of the cell, 26 (2014)).

The at least one ER helper protein may be taken for additional overexpression or engineering the host cell to additionally overexpress from Pichia pastoris (Komagataella pastoris or Komagataella phaffii), Hansenula polymorpha, Trichoderma reesei, Saccharomyces cerevisiae, Kluyveromyces lactis, Yarrowia lipolytica, Candida boidinii, Aspergillus niger, preferably from Pichia pastoris (Komagataella pastoris or Komagataella phaffii). The closest homolog from other eukaryotic species may also be taken for the at least one ER helper protein.

Preferably, said ER helper protein of the present invention, being additionally overexpressed in said host cell has an amino acid sequence as shown in SEQ ID NO: 28, or a functional homolog thereof having at least 70%, such as at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 28 (Kar2p of Pichia pastoris). Preferably, the functional homologues of the SEQ ID NO. 28 are SEQ ID NOs: 29-36. Thus, said ER helper protein of the present invention, being additionally overexpressed in said host cell has an amino acid sequence as shown in SEQ ID NOs: 28-36. The ER helper protein having the amino acid sequence as shown in SEQ ID NO. 28 is preferred. Preferably, the helper protein is not identical to the transcription factor of the present invention as indicated above and not identical to the protein of interest.

When introducing the polynucleotide encoding the at least one transcription factor under the control of a promoter by a vector or plasmid, the polynucleotide encoding the additional ER helper protein may be integrated on the same vector or plasmid under the control of the same promoter or under the control of a different promoter (Msn4p under the control of one promoter and Kar2p under the control of a different promoter). When introducing the polynucleotide encoding the at least one transcription factor under the control of a promoter by a vector or plasmid, the polynucleotide encoding the additional ER helper protein may be integrated simultaneously or consecutively (one after the other) on a different vector or plasmid. If both the polynucleotide encoding the at least one transcription factor and the polynucleotide encoding the additional ER helper protein may be introduced on different vectors or plasmids, one plasmid carrying only the at least one transcription factor and another plasmid carrying an overexpression cassette for the at least one additional ER helper protein, are preferably used.

When introducing one or more copies of the polynucleotide encoding the at least one transcription factor under the control of a promoter by a vector or plasmid, the polynucleotide encoding the additional ER helper protein may be integrated on the same vector or plasmid under the control of the same promoter or under the control of a different promoter (one or more copies of Msn4p under the control of one promoter and one or more copies of Kar2p under the control of a different promoter). When introducing one or more copies of the polynucleotide encoding the at least one transcription factor under the control of a promoter by a vector or plasmid, the polynucleotide encoding the additional ER helper protein may be integrated simultaneously or consecutively (one after the other) on a different vector or plasmid.

It is presumed, that the overexpression of the additional ER helper protein may make sure that the POI is folded correctly in the ER, thereby increasing the yield of the POI even more.

The overexpression of said Msn4p transcription factor(s) of the present invention and said first Kar2p helper protein(s) may increase the yield of the model protein compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500. The overexpression of the native (homolog) transcription factor Msn4p of P. pastoris of the present invention and of said first ER helper protein Kar2p of P. pastoris may increase the yield of the model protein, preferably of vHH (SEQ ID NO. 14) compared to the host cell prior to engineering by at least 40%, such as 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the synthetic transcription factor synMsn4p of the present invention and of said first ER helper protein Kar2p of P. pastoris may increase the yield of the model protein, preferably of vHH (SEQ ID NO. 14) to the host cell prior to engineering by at least 30%, such as 40%, 50%, 60%, 70%, 80%, 90%, 100, 120, 130, 140%, 150%, 160%, 170%, 180%, 190%, 200%, 250%, 300%, 350%, 400%, or 500%.

The methods, the recombinant host cell and the use of the present invention may comprise further overexpressing in said host cell or engineering said host cell to overexpress at least two polynucleotides encoding at least two ER helper proteins.

If the present invention refers to two additional ER helper proteins this means a “first ER helper protein” and a “second ER helper protein”. If the present invention refers to three additional ER helper proteins this means a “first ER helper protein” and a “second ER helper protein” and a “third ER helper protein”. Preferably, by further overexpressing in said host cell at least two polynucleotides encoding at least two ER helper proteins the yield of said recombinant protein of interest increases in comparison to a host cell overexpressing at least one polynucleotide encoding at least one transcription factor but not further overexpressing at least two polynucleotides encoding at least two ER helper proteins. Also preferred is by further overexpressing in said host cell at least two polynucleotides encoding at least two ER helper proteins, the yield of said recombinant protein of interest increases in comparison to a host cell overexpressing at least one polynucleotide encoding at least one transcription factor and overexpressing at least one polynucleotide encoding at least one additional ER helper protein but not overexpressing at least two polynucleotides encoding at least two ER helper proteins.

Preferably, the first ER helper protein has an amino acid sequence as shown in SEQ ID NO: 28 as mentioned above or a functional homologue thereof having at least 70%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to the amino acid sequence as shown in SEQ ID NO: 28 (Kar2p of Pichia pastoris). Preferably, the functional homologues of SEQ ID NO. 28 as the first ER helper protein additionally overexpressed to said transcription factor are SEQ ID NOs: 29-36. Thus, said first ER helper protein of the present invention, being additionally overexpressed in said host cell has an amino acid sequence as shown in SEQ ID NOs: 28-36. SEQ ID NO. 28 for the first ER helper protein is preferred.

Preferably, the second ER helper protein has an amino acid sequence as shown in SEQ ID NO: 37, or a functional homologue thereof having at least 25%, such as 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to the amino acid sequence as shown in SEQ ID NO: 37 (Lhs1p of Pichia pastoris). Thus, the present invention comprises the overexpression of a combination of the transcription factor of the present invention with the first helper protein according to SEQ ID NO. 28 (Kar2p of Pichia pastoris). or a functional homologue thereof and the second ER helper protein according to SEQ ID NO: 37 (Lhs1p of Pichia pastoris) or a functional homologue thereof. Preferably, the functional homologues of SEQ ID NO. 37 as the second ER helper protein additionally overexpressed to said transcription factor and to the first ER helper protein are SEQ ID NOs: 38-46.

The second ER helper protein having an amino acid sequence as shown in SEQ ID NO: 37 or a functional homolog thereof may be taken for additional overexpression or engineering the host cell to additionally overexpress from Pichia pastoris (Komagataella pastoris or Komagataella phaffii), Hansenula polymorpha, Trichoderma reesei, Saccharomyces cerevisiae, Kluyveromyces lactis, Yarrowia lipolytica, Candida boidinii, Schizosaccharomyces pombe, Aspergillus niger, preferably from Pichia pastoris (Komagataella pastoris or Komagataella phaffii).

The overexpression of said Msn4p transcription factor(s) of the present invention and said first Kar2p helper protein(s) and said second Lhs1p helper protein(s) may increase the yield of the model protein, preferably of scFv (SEQ ID NO. 13) and/or vHH (SEQ ID NO. 14) compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the native transcription factor Msn4p of P. pastoris of the present invention and of said first ER helper protein Kar2p of P. pastoris and of said second helper protein Lhs1p of P. pastoris may increase the yield of the model protein, preferably of vHH (SEQ ID NO. 14) compared to the host cell prior to engineering by at least 60%, such as 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the synthetic transcription factor synMsn4p of the present invention and of said first ER helper protein Kar2p of P. pastoris and of said second helper protein Lhs1p of P. pastoris may increase the yield of the model protein, preferably of scFv (SEQ ID NO. 13) compared to the host cell prior to engineering by at least 80%, such as 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%.

The present invention comprises another overexpression of a combination of the transcription factor of the present invention with the first helper protein according to SEQ ID NO. 28 or a functional homologue thereof and another second ER helper protein according to SEQ ID NO: 47 or a functional homologue thereof.

Preferably, the other second ER helper protein has an amino acid sequence as shown in SEQ ID NO. 47, or a homologue thereof, wherein the homologue has at least 20%, such as such 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to the amino acid sequence as shown in SEQ ID NO. 47 (Sil1p of Pichia pastoris). Preferably, the functional homologues of SEQ ID NO. 47 as the other second ER helper protein additionally overexpressed to said transcription factor and the first ER helper protein are SEQ ID NOs: 48-54.

The second ER helper protein having an amino acid sequence as shown in SEQ ID NO: 47 or a functional homolog thereof may be taken for additional overexpression or engineering the host cell to a additionally overexpress from Pichia pastoris (Komagataella pastoris or Komagataella phaffii), Hansenula polymorpha, Trichoderma reesei, Saccharomyces cerevisiae, Kluyveromyces lactis, Yarrowia lipolytica, Candida boidinii, preferably from Pichia pastoris (Komagataella pastoris or Komagataella phaffii). The closest homolog from other eukaryotic species may also be taken for the at least one ER helper protein. having an amino acid sequence as shown in SEQ ID NO: 47 or a functional homolog thereof.

The overexpression of said Msn4p transcription factor(s) of the present invention and said first Kar2p helper protein(s) and said second Sil1p helper protein(s) may increase the yield of the model protein, preferably of scFv (SEQ ID NO. 13) and/or vHH (SEQ ID NO. 14) compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%.

When introducing the polynucleotide encoding the at least one transcription factor under the control of a promoter by a vector or plasmid, the polynucleotides encoding the additional two ER helper proteins are integrated on the same vector or plasmid under the control of the same promoter or under the control of different promoters (a) Msn4p under the control of one promoter, Kar2p under the control of a different promoter and Lhs1p or Sil1p under the control of another different promoter or b) Msn4p and Kar2p under the control of the same promoter and Lhs1p or Sil1p under the control of a different promoter or c) Msn4p under the control of one promoter and Kar2p and Lhs1p or Sil1p under the control of another promoter). When introducing the polynucleotide encoding the at least one transcription factor under the control of a promoter by a vector or plasmid, the polynucleotides encoding the additional two ER helper proteins (one polynucleotide encoding the first ER helper protein, another polynucleotide encoding the other second ER helper protein) are integrated simultaneously or consecutively (one after the other) on a separate vector or plasmid (one vector/plasmid comprising the polynucleotide encoding at least one transcription factor, another vector/plasmid comprising the polynucleotides encoding the first and the second ER helper proteins). As an example, if both the polynucleotide encoding the at least one transcription factor and the polynucleotides encoding the additional at least two ER helper proteins may be introduced on separate vectors or plasmids, the integration plasmid BB3 only carrying the at least one transcription factor under the control of promoter and another integration plasmid BB3 carrying the additional two ER helper proteins (such as Kar2p under the control of a promoter and Lhs1p or Sil1p under the control of another promoter) can be used.

When introducing one or more copies of the polynucleotide encoding the at least one transcription factor under the control of a promoter by a vector or plasmid, the polynucleotides encoding the one or more copies of the at least two additional ER helper proteins are integrated on the same vector or plasmid under the control of the same promoter or under the control of different promoters (a) one or more copies of Msn4p under the control of one promoter, one or more copies of Kar2p under the control of a different promoter and one or more copies of Lhs1p or Sil1p under the control of another different promoter or b) one or more copies of Msn4p and Kar2p under the control of the same promoter and one or more copies of Lhs1p or Sil1p under the control of a different promoter or c) one or more copies of Msn4p under the control of one promoter and one or more copies of Kar2p and Lhs1p or Sil1p under the control of another promoter). When introducing one or more copies of the polynucleotide encoding the at least one transcription factor under the control of a promoter by a vector or plasmid, the one or more copies of the polynucleotides encoding the additional two ER helper proteins (one polynucleotide encoding the first ER helper protein, another polynucleotide encoding the other second ER helper protein) are integrated simultaneously or consecutively (one after the other) on another different vector or plasmid (one vector/plasmid comprising the polynucleotide encoding at least one transcription factor, another vector/plasmid comprising the polynucleotides encoding the first and the second ER helper proteins).

The overexpression of the two additional ER helper proteins (Kar2p and Lhs1p or Kar2p and Sil1p) may make sure that the POI is folded correctly in the ER, thereby increasing the yield/titer of the POI even more. In this embodiment, the second helper protein (e.g. Lhs1p or Sil1p) may interact as a co-chaperone with the first ER helper protein (such as Kar2p) when folding the POI.

The overexpression of or the engineering of the host cell to overexpress said additional ER helper proteins (such as Kar2p, Lhs1p or Sil1p) is achieved in any ways known to a skilled person in the art as it is also described herein previously for the homologous transcription factor of the present invention or for the heterologous transcription factor of the present invention.

The present invention comprises another overexpression of a combination of the transcription factor of the present invention with the first ER helper protein according to SEQ ID NO. 28 or a functional homologue thereof and another second ER helper protein according to SEQ ID NO: 37/SEQ ID NO: 47 or a functional homologue thereof and optionally a third ER helper protein according to SEQ ID NO. 55 or a functional homologue thereof.

Preferably, the third ER helper protein has an amino acid sequence as shown in SEQ ID NO. 55, or a homologue thereof, wherein the homologue has at least 25%, such as 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to the amino acid sequence as shown in SEQ ID NO. 55 (Erj5p of Pichia pastoris). Preferably, the functional homologues of SEQ ID NO. 55 as the third ER helper protein additionally overexpressed to said transcription factor, the first ER helper protein, and the second ER helper protein are SEQ ID NOs: 56-64.

The third ER helper protein having an amino acid sequence as shown in SEQ ID NO: 55 or a functional homolog thereof is taken from Pichia pastoris (Komagataella pastoris or Komagataella phaffii), Hansenula polymorpha, Trichoderma reesei, Saccharomyces cerevisiae, Kluyveromyces lactis, Yarrowia lipolytica, Candida boidinii, Schizosaccharomyces pombe, Aspergillus niger, preferably from Pichia pastoris (Komagataella pastoris or Komagataella phaffii).

When introducing the polynucleotide encoding the at least one transcription factor under the control of a promoter by a vector or plasmid, the polynucleotides encoding the additional three ER helper proteins are integrated on the same vector or plasmid under the control of the same promoter or under the control of different promoters. When introducing the polynucleotide encoding the at least one transcription factor under the control of a promoter by a vector or plasmid, the polynucleotides encoding the additional three ER helper proteins (one polynucleotide encoding the first ER helper protein, another polynucleotide encoding the other second ER helper protein and another polynucleotide encoding the other third ER helper protein) are integrated simultaneously or consecutively (one after the other) on another different vector or plasmid (one vector/plasmid comprising the polynucleotide encoding at least one transcription factor, another vector/plasmid comprising the polynucleotides encoding the first, the second and the third ER helper proteins). Exemplarily, if both the polynucleotide encoding the at least one transcription factor and the polynucleotides encoding the additional three ER helper proteins may be introduced on different vectors or plasmids, the integration plasmid BB3 only carrying the at least one transcription factor under the control of a promoter and another integration plasmid BB3 carrying the additional three ER helper proteins (such as Kar2p under the control of a promoter and Lhs1p or Sil1p under the control of another promoter and Erj5p under the control of again another promoter can be used.

When introducing one or more copies of the polynucleotide encoding the at least one transcription factor under the control of a promoter by a vector or plasmid, the polynucleotides encoding the one or more copies of the additional three ER helper proteins are integrated on the same vector or plasmid under the control of the same promoter or under the control of different promoters. When introducing one or more copies of the polynucleotide encoding the at least one (homologous and/or heterologous) transcription factor under the control of a promoter by a vector or plasmid, the one or more copies of the polynucleotides encoding the additional three ER helper proteins (one polynucleotide encoding the first ER helper protein, another polynucleotide encoding the other second ER helper protein and another polynucleotide encoding the third ER helper protein) are integrated simultaneously or consecutively (one after the other) on another different vector or plasmid (one vector/plasmid comprising the polynucleotide encoding at least one transcription factor, another vector/plasmid comprising the polynucleotides encoding the first, the second and the third ER helper proteins).

The overexpression of said Msn4p transcription factor(s) of the present invention and said first Kar2p helper protein(s) and said second Lhs1p helper protein(s) and said third Erj5p helper protein(s) may increase the yield of the model protein, preferably of scFv (SEQ ID NO. 13) and/or vHH (SEQ ID NO. 14) compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the native transcription factor Msn4p of P. pastoris of the present invention and of said first ER helper protein Kar2p of P. pastoris and of said second ER helper protein Lhs1p of P. pastoris and of said third ER helper protein Erj5p of P. pastoris may increase the yield of the model protein, preferably of the vHH (SEQ ID NO. 14) compared to the host cell prior to engineering by at least 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the synthetic transcription factor synMsn4p of the present invention and of said first ER helper protein Kar2p of P. pastoris and of said second ER helper protein Lhs1p of P. pastoris and of said third ER helper protein Erj5p of P. pastoris may increase the yield of the model protein, preferably of the vHH (SEQ ID NO. 14) compared to the host cell prior to engineering by at least 70%, such as 80%, 90%, 100%, 110%, 120%, 130%, 140%, 150%, 160, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%.

The overexpression of said Msn4p transcription factor(s) of the present invention and said first Kar2p helper protein(s) and said second Sil1p helper protein(s) and said third Erj5p helper protein(s) may increase the yield of the model protein scFv (SEQ ID NO. 13) and/or vHH (SEQ ID NO. 14) compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%.

The methods, the recombinant host cell and the use of the present invention may comprise further overexpressing in said host cell or engineering said host cell to overexpress at least one polynucleotide encoding one additional transcription factor. Thus, the host cell overexpresses the at least one polynucleotide encoding the at least one transcription factor of the present invention and one additional transcription factor. Preferably, by further overexpressing in said host cell at least one polynucleotide encoding at least one additional transcription factor, the yield of said recombinant protein of interest increases in comparison to a host cell overexpressing at least one polynucleotide encoding at least one transcription factor but not overexpressing at least one polynucleotide encoding at least one additional transcription factor.

The additional transcription factor was originally isolated from Pichia pastoris (Komagataella phaffi) CBS7435 strain (CBS-KNAW culture collection). It is envisioned that the transcription factor(s) can be overexpressed over a wide range of host cells. Thus, instead of using the sequences native to the species or the genus, the transcription factor sequence(s) may also be taken or derived from other prokaryotic or eukaryotic organisms. Preferably, the transcription factor(s) is/are taken for additional overexpression or engineering the host cell to additionally overexpress from Pichia pastoris (Komagataella pastoris or Komagataella phaffii), Hansenula polymorpha, Trichoderma reesei, Saccharomyces cerevisiae, Kluyveromyces lactis, Yarrowia lipolytica, Candida boidinii, and Aspergillus niger.

In the present invention the additional Hac1 transcription factor refers to SEQ ID NO. 74-82 comprising a DNA binding domain comprising an amino acid sequence as shown in SEQ ID NO: 65 or a functional homolog of the amino acid sequence as shown in SEQ ID NO: 65 having at least 50% sequence identity to the amino acid sequence as shown in SEQ ID NO: 65 as described herein and any activation domain (synthetic, viral or an activation domain of the additional transcription factor of any species as described elsewhere herein). The arrangement of said DNA binding domain of the additional transcription factor as described herein and any activation domain may be performed according to the skilled person's knowledge and may be performed in any order.

Preferably, the additional transcription factor comprises at least a DNA binding domain and an activation domain, wherein the DNA binding domain comprises an amino acid sequence as shown in SEQ ID NO: 65 (DNA binding domain of Hac1p of P. pastoris).

Preferably, the additional transcription factor comprises at least a DNA binding domain and an activation domain, wherein the DNA binding domain comprises a functional homolog of the amino acid sequence as shown in SEQ ID NO: 65 having at least 50%, such as at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to the amino acid sequence as shown in SEQ ID NO: 65.

Preferably, the functional homologs of the amino acid sequence as shown in SEQ ID NO. 65 having at least 50% sequence identity to an amino acid sequence as shown in SEQ ID NO: 65 are SEQ ID NOs: 66-73.

Thus, the method, the recombinant host cell and the use of the present invention may comprise further overexpressing an additional transcription factor comprising at least a DNA binding domain comprising an amino acid sequence as shown in SEQ ID NOs: 65-73 an activation domain.

HAC1 encodes a transcription factor of the basic leucine zipper (bZIP) family that is involved in the unfolded protein response (Mori K et al., Genes Cells 1(9):803-17, 1996 and Cox J S and Water P, Cell 87(3):391-404, 1996). Heat stress, drug treatment, mutations in secretory proteins, or overexpression of wild type secretory proteins can cause unfolded proteins to accumulate in the ER, triggering the unfolded protein response (UPR). HAC1 is not essential under normal growth conditions, but is essential under conditions that trigger the UPR. Hac1p binds to a DNA sequence called the UPR element (UPRE) in the promoter of UPR-regulated genes such as KAR2, PDI1, EUG1, FKB2. The abundance of Hac1p is regulated by splicing of the HAC1 mRNA. The spliced HAC1 mRNA is translated much more efficiently than the unspliced transcript. Hac1p induces the transcription of genes encoding ER chaperons such as Kar2p for example being involved in the UPR. Increased transcription of genes encoding soluble ER resident proteins, including ER chaperones for example, is a key feature of the UPR. Further, Hac1p increases synthesis of ER-resident proteins required for protein folding.

When introducing the polynucleotide encoding the at least one transcription factor under the control of a promoter by a vector or plasmid, the polynucleotide encoding the additional transcription factor is integrated on the same vector or plasmid under the control of the same promoter or under the control of a different promoter (Msn4p under the control of one promoter, Hac1p under the control of a different promoter). If both the polynucleotide encoding the at least one transcription factor and the polynucleotide encoding the additional transcription factor may be introduced on the same vector or plasmid, an integration plasmid BB3 is preferably used, wherein the polynucleotide encoding the at least one transcription factor is under the control of a promoter and the polynucleotide encoding the at least one additional transcription factor is under the control of a different promoter. When introducing the polynucleotide encoding the at least one transcription factor under the control of a promoter by a vector or plasmid, the polynucleotides encoding the additional transcription factor is integrated simultaneously or consecutively (one after the other) on a different vector or plasmid. As an example, if both the polynucleotide encoding the at least one transcription factor and the polynucleotide encoding the additional transcription factor may be introduced on different vectors or plasmids, an integration plasmid BB3 only carrying the at least one transcription factor and another integration plasmid BB3 only carrying the at least one additional transcription factor can be used.

When introducing one or more copies of the polynucleotide encoding the at least one transcription factor under the control of a promoter by a vector or plasmid, the one or more copies of the polynucleotide encoding the additional transcription factor is integrated on the same vector or plasmid under the control of the same promoter or under the control of a different promoter (one or more copies of Msn4p under the control of one promoter, one or more copies of Hac1p under the control of a different promoter). When introducing one or more copies of the polynucleotide encoding the at least one transcription factor under the control of a promoter by a vector or plasmid, the one or more copies of the polynucleotide encoding the additional transcription factor is integrated simultaneously or consecutively (one after the other) on a different vector or plasmid.

The overexpression of the additional transcription factor may result in the overexpression of ER chaperones for example Kar2p being a key feature of the UPR, thereby increasing the yield of the POI even more.

The overexpression of said Msn4p transcription factor(s) of the present invention and said Hac1p additional transcription factor(s) may increase the yield of the model protein scFv (SEQ ID NO. 13) and/or vHH (SEQ ID NO. 14) compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500. The overexpression of the native transcription factor Msn4p of P. pastoris of the present invention and of said Hac1p additional transcription factor of P. pastoris may increase the yield of the model protein, preferably of the vHH (SEQ ID NO. 14) compared to the host cell prior to engineering by at least 60%, such as 70%, 80%, 90%, 100%, 110%, 120%, 130%, 140%, 150, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the synthetic transcription factor synMsn4p of the present invention and of said Hac1p additional transcription factor of P. pastoris may increase the yield of the model protein, preferably of the vHH (SEQ ID NO. 14) compared to the host cell prior to engineering by at least 80%, such as 90%, 100%, 110%, 120%, 130%, 140%, 150, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%.

Said at least one polynucleotide encoding the at least one additional transcription factor encodes for a heterologous or homologous additional transcription factor. The overexpression of or the engineering of the host cell to overexpress said additional transcription factor (Hac1p) is achieved as discussed previously for the homologous transcription factor of the present invention or for the heterologous transcription factor of the present invention.

The additional transcription factor(s) used in the methods, the recombinant host cell and the use of the present invention may comprise an amino acid sequence as shown in SEQ ID NOs: 74-82 or a functional homolog of the amino acid sequence as shown in SEQ ID NO 74 having at least 20% sequence identity of the amino acid sequence as shown in SEQ ID NO 74. In a further embodiment, the additional transcription factor(s) used in the methods, the recombinant host cell and the use of the present invention may comprise an amino acid sequence as shown in SEQ ID NOs: 74-82 or a functional homolog of the amino acid sequence as shown in SEQ ID NO 74 having at least 20%, such as 25%, 30%, 35%, 40%, 45%, 50%, 55%, 60%, 65%, 70%, 75%, 80%, 85%, 90%, 95%, 98% or even 100% sequence identity of the amino acid sequence as shown in SEQ ID NO 74. The additional transcription factor(s) may additionally comprise a nuclear localization signal (NLS).

The present invention further envisages a method of increasing secretion of a recombinant protein of interest by a eukaryotic host cell, comprising overexpressing in said host cell at least one polynucleotide encoding at least one transcription factor, thereby increasing the yield of said recombinant protein of interest in comparison to a host cell which does not overexpress the polynucleotide encoding said transcription factor, wherein the transcription factor comprises at least a DNA binding domain comprising an amino acid sequence as shown in SEQ ID NO: 1 and an activation domain.

Further, the present invention further envisages a method of increasing secretion of a recombinant protein of interest by a eukaryotic host cell, comprising overexpressing in said host cell at least one polynucleotide encoding at least one transcription factor, thereby increasing the yield of said recombinant protein of interest in comparison to a host cell which does not overexpress the polynucleotide encoding said transcription factor, wherein the transcription factor comprises at least a DNA binding domain comprising a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87 and an activation domain.

The present invention also provides a recombinant eukaryotic host cell for manufacturing a protein of interest, wherein the host cell is engineered to overexpress at least one polynucleotide encoding at least one transcription factor.

Preferably, the present invention provides a recombinant eukaryotic host cell for manufacturing a protein of interest, wherein the host cell is engineered to overexpress at least one polynucleotide encoding at least one transcription factor, wherein the transcription factor comprises at least a DNA binding domain and an activation domain, wherein the DNA binding domain comprises an amino acid sequence as shown in SEQ ID NO. 1.

Further, the present invention provides a recombinant eukaryotic host cell for manufacturing a protein of interest, wherein the host cell is engineered to overexpress at least one polynucleotide encoding at least one transcription factor, wherein the transcription factor comprises at least a DNA binding domain comprising a functional homolog of the amino acid sequence as shown in SEQ ID NO: 1 having at least having at least 60% sequence identity to the amino acid sequence as shown in SEQ ID NO: 1 and/or having at least 60% sequence identity to an amino acid sequence as shown in SEQ ID NO: 87 and an activation domain.

A “recombinant cell” or “recombinant host cell” refers to a cell or host cell that has been genetically altered to comprise a nucleic acid sequence which was not native to said cell.

The present invention further encompasses the use of the recombinant eukaryotic host cell for manufacturing a recombinant protein of interest. The host cells can be advantageously used for introducing polypeptides encoding one or more POI(s), and thereafter can be cultured under suitable conditions to express the POI.

### EXAMPLES

The following examples are put forth to provide those of ordinary skill in the art with a complete disclosure and description of how to make and use the subject invention, and are not intended to limit the scope of what is regarded as the invention and defined in the claims. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

The examples below will demonstrate that the newly identified helper protein(s) increase(s) the titer (product per volume in mg/L) and the yield (product per biomass in mg/g biomass measured as dry cell weight or wet cell weight), respectively, of recombinant proteins upon its/their overexpression. As an example, the yield of recombinant antibody single chain variable fragments (scFv, vHH) in the yeast Pichia pastoris are increased. The positive effect was shown in shaking cultures (conducted in shake flasks or deep well plates) and in lab scale fed-batch cultivations.

### Example 1: Construction and Selection of P. pastoris Strains Secreting Antibody Fragments scFv & vHH

P. pastoris CBS7435 mut^{s} variant (genome sequenced by Sturmberger et al. 2016) was used as host strain. The pPM2d_pGAP and pPM2d_pAOX expression plasmids are derivatives of the pPuzzle_ZeoR plasmid backbone described in WO2008/128701A2, consisting of the pUC19 bacterial origin of replication and the Zeocin antibiotic resistance cassette. Expression of the heterologous gene is mediated by the P. pastoris glyceraldehyde-3-phosphate dehydrogenase (GAP) promoter or alcohol oxidase (AOX) promoter, respectively, and the S. cerevisiae CYC1 transcription terminator. The plasmids already contained the N-terminal S. cerevisiae alpha mating factor pre-pro leader sequence. The genes for the scFv and vHH were codon-optimized by DNA2.0 and obtained as synthetic DNA. A His6-tag was fused C-terminally to the genes for detection. After restriction digest with XhoI and BamHI (for scR) or EcoRV (for vHH), each gene was ligated into both plasmids pPM2d_pGAP and pPM2d_pAOX digested with XhoI and BamHI or EcoRV.

Plasmids were linearized using AvrI1 restriction enzyme (for pPM2d_pGAP) or PmeI restriction enzyme (for pPM2d_pAOX), respectively, prior to electroporation (using a standard transformation protocol as described in Gasser et al. 2013. Future Microbiol. 8(2):191-208) into P. pastoris. Selection of positive transformants was performed on YPD plates (per liter: 10 g yeast extract, 20 g peptone, 20 g glucose, 20 g agar-agar) containing 100 μg/mL of Zeocin.

Single colonies (in total ˜120) of all transformation approaches were picked from transformation plates into single wells of 96-deep well plates. After an initial growth phase to generate biomass, expression from the AOX1 promoter was induced by supplementation with a media formulation containing methanol (4 times in total). After 72 hours from first methanol induction, all deep well plates were centrifuged and supernatants of all wells were harvested into stock microtiter plates for subsequent analysis. Expression from the GAP promoter was continued by supplementation of glucose at defined points of time (i.e. twice per day for 2 days) after the initial growth phase. After a total of 110 hours from the initial inoculation, cultures were harvested as above.

The clones with the highest productivities in small scale screenings (Example 3) and fed batch cultivations (Example 4) were selected to be the basic production strains for further engineering. The clone CBS7435 mut^{s} pAOX scR 4E3 was selected as basic production strain for scFv secretion. The clone CBS7435 mut^{s} pAOX vHH 14G8 was selected as basic production strain for vHH secretion.

### Example 2: Generation of Engineered Strains Overexpressing Helper Genes

For the investigation of positive effects on scFv and vHH secretion, the putative helper genes were overexpressed in the two basic production strains: CBS7435 mut^{s} pAOX scR (scFv) 4E3 and CBS7435 mut^{s} pAOX vHH (vHH) 14G8 (generation see Example 1).

a) General Procedure of Amplification and Cloning of the Selected Potential Secretion Helper Genes

The genes selected for overexpression were amplified by PCR (Q5® High-Fidelity DNA Polymerase, New England Biolabs) from start to stop codon or split into two several fragments. The GoldenPiCS system (Prielhofer et al. 2017. BMC Systems Biol. doi: 10.1186/s12918-017-0492-3) requires the introduction of silent mutations in some coding sequences. This was performed by amplifying several fragments from one coding sequence. Alternatively, gBlocks or synthetic codon-optimized genes were obtained from commercial providers (including Integrated DNA Technology IDT, Geneart, and ATUM). Amplified coding sequences were either cloned into the pPUZZLE-based expression plasmids pPM2aK21 or pPM2eH21, or the GoldenPiCS system (consisting of the backbones BB1, BB2 and BB3aK/BB3eH/BB3rN). The gene fragments listed in Table 1 were introduced into BB1 of the GoldenPiCS system by using the restriction enzyme BsaI. All promoters and terminators used to assemble expression cassettes in BB2 or BB3 backbones are described in Prielhofer et al. 2017. (BMC Systems Biol. doi: 10.1186/s12918-017-0492-3). pPM2aK21 and BB3aK allow integration into the 3″-AOX1 genomic region and contain the KanMX selection marker cassette for selection in E. coli and yeast. pPM2eH21 and BB3eH contain the 5″-ENO1 genome integration region and the HphMX selection marker cassette for selection on hygromycin. BB3rN contain the 5″-RGI1 genome integration region and the NatMX selection marker cassette for selection on nourseothricin. All plasmids contain an origin of replication for E. coli (pUC19). Genomic DNA from P. pastoris strain CBS7435 mut^{s} or gBlocks (Integrated DNA Technologies) served as PCR templates.

Table 1 lists the required gene fragments for introducing them into the BB1 of the GoldenPiCS system by using the restriction enzyme BsaI. The assembled BB1s carrying the respective coding sequence were then further processed in the GoldenPiCS system to create the required BB3 integration plasmids as described in Prielhofer et al. 2017. The underlined nucleotides mark the first forward and the last reverse primer required to create the GoldenPiCS compatible gene fragment, start and stop codon are marked in bold.

| Gene | Gene | |
|---|---|---|
| identifier | fragment | Cloned sequence |
| PP7435_ | MSN4 | GATAGGTCTCTCATGTCTACAACAAAACCAATGCAGGTGTTAGCCCCGGACCTTACTGA |
| Chr2- | | GACACCAAAGACATATTCGTTAGGTGTCCATTTGGGGAAAGGCAAGGACAAACTCCAG |
| 0555 | | GATCCGACAGAACTCTACTCGATGATCCTAGATGGAATGGATCACTCACAGCTCAATTC |
| | | TTTTATTAACGATCAGTTGAACTTGGGATCATTGCGCTTGCCGGCGAATCCTCCTGCTG |
| | | CAAGTGGTGCTAAACGGGGTGCAAATGTCAGTTCTATCAACATGGATGATTTACAAACG |
| | | TTTGATTTCAACTTTGATTACGAACGGGATTCATCGCCGCTAGAATTGAACATGGATTCT |
| | | CAATCTTTGATGTTTTCCTCTCCAGAGAAAGCTCCCTGTGGCTCCTTGCCGTCTCAGCA |
| | | TCAGCCTCACTCTCAGGTCGCAGCCGCACAGGGAACTACCATCAATCCAAGGCAGTTA |
| | | TCCACATCTTCTGCCAGTAGCTTTGTATCTTCGGATTTTGATGTTGATTCACTCCTGGCA |
| | | GACGAGTACGCTGAGAAACTAGAATATGGAGCCATATCATCTGCCTCATCTTCCATCTG |
| | | TTCGAATTCTGTTCTTCCTAGCCAGGGCGTAACTTCGCAACATAGCTCTCCTATAGAAC |
| | | AAAGACCTCGTGTGGGAAATTCCAAACGCTTGAGTGATTTTTGGATGCAGGACGAAGCT |
| | | GTCACTGCCATTTCCACCTGGCTCAAAGCTGAAATACCTTCCTCCTTGGCTACGCCGGC |
| | | TCCTACAGTCACACAAATAAGTAGTCCCAGCCTTAGCACCCCAGAGCCAAGGAAGAAA |
| | | GAAACAAAACAAAGAAAGAGGGCAAAGTCCATAGACACGAATGAGCGATCTGAACAAG |
| | | TAGCAGCTTCTAATTCAGATGATGAAAAGCAATTCCGCTGCACGGATTGCAGTAGACGC |
| | | TTCCGCAGATCAGAACACCTGAAACGACATCATAGGTCTGTTCATTCTAACGAAAGGCC |
| | | GTTCCATTGTGCTCACTGTGATAAACGGTTCTCAAGAAGCGACAACTTGTCGCAGCATC |
| | | TACGTACTCACCGTAAGCAGTGAGCTTAGAGACCTATC |
| | | (SEQ ID NO: 88) |
| PP7435_ | MSN4 | 5′-GATAGGTCTCTCATGTCTACAACAAAACCAATGCAG-3′ |
| Chr2- | | (SEQ ID NO: 89) |
| 0555 | | 5′-GATAGGTCTCTAAGCTCACTGCTTACGGTGAGTAC-3′ |
| | | (SEQ ID NO: 90) |
| n.a. | synMSN4 | GATCTAGGTCTCACATGGGTAAGCCAATTCCTAACCCATTGTTGGGTTTGGATTCTACT |
| | | CCAAAAAAGAAGAGAAAGGTTGGTGGAGGTGGATCTgatgcccttgacgattttgacttggacatgttgg |
| | | gttctgacgctttggatgactttgatcttgatatgcttggttccgacgctctagatgatttcgacttgga |
| | | tatgctgggatccgatgccttggacgatttcgacttggatatgttgGGTGGAGGTGGATCTAATTCAGAT |
| | | GATGAAAAGCAATTCCGCTGCACGGATTGCAGTAGACGCTTCCGCAGATCAGAACACCTGAAACGACATC |
| | | ATAGGTCTGTTCATTCTAACGAAAGGCCGTTCCATTGTGCTCACTGTGATAAACGGTTCTCAAGAAGCGA |
| | | CAACTTGTCGCAGCATCTACGTACTCACCGTAAGCAGTGATAGGCTTCGAGACCAATGAC |
| | | (SEQ ID NO: 91) |
| n.a. | synMSN4 | 5′-GATCTAGGTCTCACATGGGTAAGCCAATTCCTAACC-3′ |
| | | (SEQ ID NO: 92) |
| | | 5′-GTCATTGGTCTCGAAGCCTATCACTGCTTACGGTGAG-3′ |
| | | (SEQ ID NO: 93) |
| YMR037C | S. cerevisiae | GATAGGTCTCGCATGACGGTCGACCATGATTTCAATAGCGAAGATATTTTATTCCCCAT |
| | MSN2 | AGAAAGCATGAGTAGTATACAATACGTGGAGAATAATAACCCAAATAATATTAACAACGA |
| | | TGTTATCCCGTATTCTCTAGATATCAAAAACACTGTCTTAGATAGTGCGGATCTCAATGA |
| | | CATTCAAAATCAAGAAACTTCACTGAATTTGGGGCTTCCTCCACTATCTTTCGACTCTCC |
| | | ACTGCCCGTAACGGAAACGATACCATCCACTACCGATAACAGCTTGCATTTGAAAGCTG |
| | | ATAGCAACAAAAATCGCGATGCAAGAACTATTGAAAATGATAGTGAAATTAAGAGTACTA |
| | | ATAATGCTAGTGGCTCTGGGGCAAATCAATACACAACTCTTACTTCACCTTATCCTATGA |
| | | ACGACATTTTGTACAACATGAACAATCCGTTACAATCACCGTCACCTTCATCGGTACCTC |
| | | AAAATCCGACTATAAATCCTCCCATAAATACAGCAAGTAACGAAACTAATTTATCGCCTC |
| | | AAACTTCAAATGGTAATGAAACTCTTATATCTCCTCGAGCCCAACAACATACGTCCATTA |
| | | AAGATAATCGTCTGTCCTTACCTAATGGTGCTAATTCGAATCTTTTCATTGACACTAACC |
| | | CAAACAATTTGAACGAAAAACTAAGAAATCAATTGAACTCAGATACAAATTCATATTCTAA |
| | | CTCCATTTCTAATTCAAACTCCAATTCTACGGGTAATTTAAATTCCAGTTATTTTAATTCA |
| | | CTGAACATAGACTCCATGCTAGATGATTACGTTTCTAGTGATCTCTTATTGAATGATGAT |
| | | GATGATGACACTAATTTATCACGCCGAAGATTTAGCGACGTTATAACAAACCAATTTCCG |
| | | TCAATGACAAATTCGAGGAATTCTATTTCTCACTCTTTGGACCTTTGGAACCATCCGAAA |
| | | ATTAATCCAAGCAATAGAAATACAAATCTCAATATCACTACTAATTCTACCTCAAGTTCCA |
| | | ATGCAAGTCCGAATACCACTACTATGAACGCAAATGCAGACTCAAATATTGCTGGCAAC |
| | | CCGAAAAACAATGACGCTACCATAGACAATGAGTTGACACAGATTCTTAACGAATATAAT |
| | | ATGAACTTCAACGATAATTTGGGCACATCCACTTCTGGCAAGAACAAATCTGCTTGCCC |
| | | AAGTTCTTTTGATGCCAATGCTATGACAAAGATAAATCCAAGTCAGCAATTACAGCAACA |
| | | GCTAAACCGAGTTCAACACAAGCAGCTCACCTCGTCACATAATAACAGTAGCACTAACA |
| | | TGAAATCCTTCAACAGCGATCTTTATTCAAGAAGGCAAAGAGCTTCTTTACCCATAATCG |
| | | ATGATTCACTAAGCTACGACCTGGTTAATAAGCAGGATGAAGATCCCAAGAACGATATG |
| | | CTGCCGAATTCAAATTTGAGTTCATCTCAACAATTTATCAAACCGTCTATGATTCTTTCAG |
| | | ACAATGCGTCCGTTATTGCGAAAGTGGCGACTACAGGCTTGAGTAATGATATGCCATTT |
| | | TTGACAGAGGAAGGTGAACAAAATGCTAATTCTACTCCAAATTTCGATCTTTCCATCACT |
| | | CAAATGAATATGGCTCCATTATCGCCTGCATCATCATCCTCCACGTCTCTTGCAACAAAT |
| | | CATTTCTATCACCATTTCCCACAGCAGGGTCACCATACCATGAACTCTAAAATCGGTTCT |
| | | TCCCTTCGGAGGCGGAAGTCTGCTGTGCCTTTGATGGGTACGGTGCCGCTTACAAATC |
| | | AACAAAATAATATAAGCAGTAGTAGTGTCAACTCAACTGGCAATGGTGCTGGGGTTACG |
| | | AAGGAAAGAAGGCCAAGTTACAGGAGAAAATCAATGACACCGTCCAGAAGATCAAGTG |
| | | TCGTAATAGAATCAACAAAGGAACTCGAGGAGAAACCGTTCCACTGTCACATTTGTCCC |
| | | AAGAGCTTTAAGCGCAGCGAACATTTGAAAAGGCATGTGAGATCTGTTCACTCTAACGA |
| | | ACGACCATTTGCTTGTCACATATGCGATAAGAAATTTAGTAGAAGCGATAATTTGTCGCA |
| | | ACACATCAAGACTCATAAAAAACATGGAGACATTTAAGCTTGGAGACCTATC |
| | | (SEQ ID NO: 94) |
| YMR037C | S. cerevisiae | 5′-GATAGGTCTCGCATGACGGTCGACCATG-3′ |
| | MSN2 | (SEQ ID NO: 95) |
| | | 5′-GATAGGTCTCCAAGCTTAAATGTCTCCATGTTTTTTATGAGT-3′ |
| | | (SEQ ID NO: 96) |
| YKL062W | S. cerevisiae | GACTGGTCTCACATGCTAGTCTTTGGACCTAATAGTAGTTTCGTTCGTCACGCAAACAA |
| | MSN4 | GAAACAAGAAGATTCGTCTATAATGAACGAGCCAAACGGATTGATGGACCCGGTATTGA |
| | | GCACAACCAACGTTTCTGCTACTTCTTCTAATGACAATTCTGCGAACAATAGCATATCTT |
| | | CGCCGGAATATACCTTTGGTCAATTCTCAATGGATTCTCCGCATAGAACGGACGCCACT |
| | | AATACTCCAATTTTAACAGCGACAACTAATACGACTGCTAATAATAGTTTAATGAATTTAA |
| | | AGGATACCGCCAGTTTAGCTACCAACTGGAAGTGGAAAAATTCCAATAACGCACAGTTC |
| | | GTGAATGACGGTGAGAAACAAAGCAGTAATGCTAATGGTAAGAAAAATGGTGGTGATAA |
| | | GATATATAGTTCAGTAGCCACCCCTCAAGCTTTAAATGACGAATTGAAAAACTTGGAGC |
| | | AACTAGAAAAGGTATTTTCTCCAATGAATCCTATCAATGACAGTCATTTTAATGAAAATAT |
| | | AGAATTATCGCCACACCAACATGCAACTTCTCCCAAGACAAACCTTCTTGAGGCAGAAC |
| | | CTTCAATATATTCCAATTTGTTTCTAGATGCTAGGTTACCAAACAACGCCAACAGTACAA |
| | | CAGGATTGAACGACAATGATTATAATCTAGACGATACCAATAATGATAATACTAATAGCA |
| | | TGCAATCAATCTTAGAGGATTTTGTATCTTCAGAAGAAGCATTGAAGTTCATGCCGGAC |
| | | GCTGGTCGCGACGCAAGAAGATACAGCGAGGTGGTTACCTCTTCCTTTCCTTCTATGAC |
| | | GGATTCTAGAAATTCGATCTCTCATTCGATAGAGTTTTGGAATCTCAATCACAAAAATAG |
| | | TAGCAACAGTAAACCCACTCAACAAATTATCCCTGAAGGTACTGCCACTACTGAGAGGC |
| | | GTGGATCAACCATTTCACCTACTACCACTATAAACAACTCTAATCCAAACTTCAAATTATT |
| | | AGATCATGACGTTTCTCAAGCTCTGAGCGGTTATAGTATGGATTTTTCTAAGGACTCTG |
| | | GTATAACAAAGCCAAAAAGCATTTCCTCTTCTTTAAATCGCATCTCCCATAGCAGTAGCA |
| | | CCACAAGGCAACAGCGTGCCTCTTTGCCCTTAATTCATGATATTGAATCTTTTGCAAATG |
| | | ATTCGGTGATGGCAAATCCTCTGTCTGATTCCGCATCATTTCTTTCAGAAGAAAATGAAG |
| | | ATGATGCTTTTGGTGCGCTAAATTACAATAGCTTAGATGCAACCACAATGTCGGCATTC |
| | | GACAATAACGTAGACCCCTTCAACATTCTCAAGTCATCTCCGGCTCAGGATCAACAGTT |
| | | TATCAAACCCTCTATGATGTTGTCGGATAATGCCTCTGCTGCCGCTAAATTGGCGACTT |
| | | CTGGTGTTGATAATATCACACCTACACCAGCTTTCCAAAGAAGAAGCTATGATATCTCGA |
| | | TGAACTCTTCGTTCAAAATACTTCCTACTAGTCAAGCTCACCATGCAGCTCAACATCATC |
| | | AACAACAACCTACTAAACAGGCAACGGTAAGCCCAAACACAAGAAGAAGAAAGTCGTCA |
| | | AGTGTTACTTTAAGTCCAACTATTTCTCATAACAACAACAATGGTAAGGTTCCTGTCCAA |
| | | CCTCGGAAAAGGAAATCTATTACTACCATTGACCCCAACAACTACGATAAAAATAAACCT |
| | | TTCAAGTGTAAAGACTGTGAGAAGGCATTCAGACGCAGTGAGCACTTGAAAAGGCATAT |
| | | AAGATCCGTTCATTCAACGGAACGCCCTTTTGCTTGTATGTTCTGTGAGAAAAAATTCAG |
| | | TAGAAGTGACAATTTATCACAACATCTAAAAACTCACAAAAAGCACGGTGATTTTTGAGC |
| | | TTGGAGACCTATC (SEQ ID NO: 97) |
| YKL062W | S. cerevisiae | 5′-GACTGGTCTCACATGCTAGTCTTTGGACCTAATAGTAG-3′ |
| | MSN4 | (SEQ ID NO: 98) |
| | | 5′-GATAGGTCTCCAAGCTCAAAAATCACCGTGCTT-3′ |
| | | (SEQ ID NO: 99) |
| YALI0B21582 | Y. lipolytica | GATAGGTCTCACATGGACCTCGAATTGGAAATTCCCGTCTTGCATTCCATGGACTCGCA |
| | MSN4 | CCACCAGGTGGTGGACTCCCACAGACTGGCACAGCAACAGTTCCAGTACCAGCAGATC |
| | | CACATGCTGCAGCAGACGCTGTCACAGCAGTACCCCCACACCCCATCCACCACACCCC |
| | | CCATTTACATGCTGTCGCCTGCGGACTACGAGAAGGACGCCGTTTCCATCTCACCGGT |
| | | AATGCTGTGGCCCCCCTCGGCCCACTCCCAGGCCTCTTACCATTACGAGATGCCCTCC |
| | | GTTATCTCGCCATCTCCTTCTCCCACTAGATCCTTCTGTAATCCGAGAGAGCTGGAGGT |
| | | TCAGGACGAGCTCGAGCAGCTTGAACAGCAGCCCGCCGCTCTCTCCGTCGAACATCTG |
| | | TTTGACATTGAGAACTCATCGATCGAGTATGCACACGACGAGCTGCATGACACCTCTTC |
| | | GTGCTCCGACTCGCAGTCGAGCTTTTCCCCTCAGCAGTCCCCTGCCTCCCCGGCCTCC |
| | | ACTTACTCGCCTCTCGAGGACGAGTTTCTCAACTTGGCTGGATCCGAGTTGAAGAGCG |
| | | AGCCCAGCGCGGACGACGAGAAGGATGATGTGGACACGGAGCTTCCCCAGCAGCCCG |
| | | AGATCATCATCCCTGTGTCGTGCCGAGGCCGAAAGCCGTCCATCGACGACTCCAAAAA |
| | | GACTTTTGTCTGCACCCACTGCCAGCGTCGGTTCCGGCGCCAGGAGCATCTCAAGCGA |
| | | CATTTCCGATCCCTACACACTCGAGAGAAGCCTTTCAACTGCGACACGTGCGGCAAGA |
| | | AGTTTTCTCGGTCGGACAATCTCGCCCAGCATATGCGTACGCATCCTCGGGACTAGGC |
| | | TTTGAGACCAGTC (SEQ ID NO: 100) |
| YALI0B21582 | Y. lipolytica | 5′-GATAGGTCTCACATGGACCTCGAATTGGA-3′ |
| | MSN4 | (SEQ ID NO: 101) |
| | | 5′-GACTGGTCTCAAAGCCTAGTCCCGAGGATGC-3′ |
| | | (SEQ ID NO: 102) |
| An04g03980 | Aspergillus | GATAGGTCTCACATGGACGGAACATACACCATGGCACCTACTTCGGTGCAAGGTCAAC |
| | niger Seb1 = | CATCATTTGCATACTACGCTGATTCGCAGCAAAGACAACATTTCACCAGCCACCCCTCA |
| | homolog of | GATATGCAGTCATACTATGGCCAAGTGCAGGCCTTCCAGCAACAACCACAGCACTGCA |
| | Msn2/4 | TGCCGGAGCAGCAGACACTCTACACTGCCCCTCTCATGAACATGCACCAGATGGCTAC |
| | | CACCAATGCCTTCCGTGGTGCCATGAACATGACTCCCATTGCCTCTCCTCAGCCGTCAC |
| | | ACCTCAAGCCCACAATTGTTGTGCAGCAGGGCTCTCCCGCCCTGATGCCTCTGGACAC |
| | | GAGGTTCGTCGGTAACGACTACTACGCATTCCCCTCCACCCCACCACTCTCCACAGCT |
| | | GGAAGCTCTATCAGCAGCCCGCCTTCTACCAGCGGCACCCTTCACACCCCGATCAATG |
| | | ACAGCTTCTTCGCTTTCGAGAAGGTGGAAGGTGTCAAGGAGGGATGCGAGGGAGACG |
| | | TCCATGCAGAGATTCTGGCCAATGCTGACTGGGCCCGGTCTGACTCGCCGCCTCTTAC |
| | | ACCTGGTAAGTCATTATCTAACCCGATGTCCCTTTTTTACATGGTTGCAAGATAGGCTGC |
| | | AGGGAGTGGGTGCAGCCAACGGAAAAGGCACGGGGCCGGGCATCTAGGGTTGTACAG |
| | | GGAGACTAACTCGACTTGTTCTAGTGTTCATCCATCCGCCTTCCCTCACCGCCAGCCAA |
| | | ACATCCGAGCTTCTGTCAGCGCACAGCTCTTGCCCATCCCTTTCCCCATCGCCATCTCC |
| | | CGTGGTCCCCACATTCGTTGCCCAGCCTCAAGGTCTGCCGACCGAGCAGTCCAGCTCC |
| | | GACTTCTGTGACCCCCGTCAGCTGACGGTTGAGTCCTCCATCAATGCCACCCCTGCTG |
| | | AGCTGCCGCCTCTGCCCACGCTCTCCTGCGATGACGAGGAGCCTCGGGTGGTTCTGG |
| | | GCAGCGAGGCCGTGACCCTTCCTGTCCATGAAACCCTCTCTCCCGCCTTCACCTGCTC |
| | | CTCTTCGGAGGACCCTCTCAGCAGCCTGCCGACCTTTGACAGCTTCTCGGACCTGGAC |
| | | TCGGAAGATGAATTCGTCAACCGCCTGGTCGACTTCCCCCCTAGTGGCAATGCCTACT |
| | | ACTTGGGTGAGAAGAGGCAGCGCGTGGGAACGACATACCCCCTTGAGGAAGAGGAAT |
| | | TCTTCAGTGAGCAGAGCTTCGACGAGTCTGACGAGCAAGATCTCTCTCAGTCCAGTCTC |
| | | CCTTACCTGGGAAGCCACGACTTCACTGGCGTCCAGACGAACATCAATGAAGCTTCGG |
| | | AAGAGATGGGCAACAAGAAGAGGAACAACCGCAAGTCGCTGAAGCGGGCTAGTACCT |
| | | CGGACAGCGAAACGGATTCGATTAGCAAGAAGTCGCAGCCTTCGATCAACAGCCGTGC |
| | | CACCAGCACTGAGACAAACGCCTCGACACCCCAGACTGTCCAGGCCCGCCACAACTCC |
| | | GATGCGCATTCGTCGTGCGCTTCTGAGGCTCCTGCTGCCCCCGTCTCGGTCAACCGAC |
| | | GCGGTCGTAAGCAGTCCCTGACGGATGACCCCTCCAAGACCTTCGTGTGCACCCTCTG |
| | | CTCCCGTCGCTTCCGTCGCCAAGAGCACCTCAAGCGTCACTACCGCTCTCTCCACACT |
| | | CAGGACAAGCCTTTCGAGTGCAATGAGTGCGGTAAGAAGTTCTCGCGGAGCGATAACC |
| | | TTGCGCAGCACGCTCGCACTCATGCGGGTGGCTCTGTCGTGATGGGCGTCATCGACA |
| | | CCGGCAATGCGACCCCGCCAACCCCCTATGAAGAACGAGATCCCAGTACGCTGGGAA |
| | | ATGTTCTCTACGAGGCCGCCAACGCCGCCGCTACCAAGTCCACAACCAGTGAGTCGGA |
| | | TGAGAGTTCCTCTGACTCGCCGGTTGCCGACCGACGGGCGCCCAAGAAGCGCAAGCG |
| | | CGACAGCGATGCCTAGGCTTGGAGACCATC (SEQ ID NO: 103) |
| An04g03980 | Aspergillus | 5′-GATAGGTCTCACATGGACGGAACATACACC-3′ |
| | niger Seb 1 | (SEQ ID NO: 104) |
| | | 5′-GATGGTCTCCAAGCCTAGGCATCGCTGTC-3′ |
| | | (SEQ ID NO: 105) |
| PP7435_ | KAR2 | GATCTAGGTCTCCCATGCTGTCGTTAAAACCATCTTGGCTGACTTTGGCGGCATTAATG |
| Chr2- | | TATGCCATGCTATTGGTCGTAGTGCCATTTGCTAAACCTGTTAGAGCTGACGATGTCGA |
| 1167 | | ATCTTATGGAACAGTGATTGGTATCGATTTGGGTACCACGTACTCTTGTGTCGGTGTGA |
| | | TGAAGTCGGGTCGTGTAGAAATTCTTGCTAATGACCAAGGTAACAGAATCACTCCTTCC |
| | | TACGTTAGTTTCACTGAAGATGAGAGACTGGTTGGTGATGCTGCTAAGAACTTAGCTGC |
| | | TTCTAACCCAAAAAACACCATCTTTGATATTAAGAGATTGATCGGTATGAAGTATGATGC |
| | | CCCAGAGGTCCAAAGAGACTTGAAGCGTCTGCCTTACACTGTCAAGAGCAAGAACGGC |
| | | CAACCTGTCGTTTCTGTCGAGTACAAGGGTGAGGAGAAGTCTTTCACTCCTGAGGAGAT |
| | | TTCCGCCATGGTCTTGGGTAAGATGAAGTTGATCGCTGAGGACTACTTAGGAAAGAAAG |
| | | TCACTCATGCTGTCGTTACCGTTCCAGCCTACTTCAACGACGCTCAACGTCAAGCCACT |
| | | AAGGATGCCGGTCTAATCGCCGGTTTGACTGTTCTGAGAATTGTGAACGAGCCTACCG |
| | | CCGCTGCCCTTGCTTACGGTTTGGACAAGACTGGTGAGGAAAGACAGATCATCGTCTA |
| | | CGACTTGGGTGGAGGAACCTTCGATGTTTCTCTGCTTTCTATTGAGGGTGGTGCTTTCG |
| | | AGGTTCTTGCTACCGCCGGTGACACCCACTTGGGTGGTGAGGACTTTGACTACAGAGT |
| | | TGTTCGCCACTTCGTTAAGATTTTCAAGAAGAAGCATAACATTGACATCAGCAACAATGA |
| | | TAAGGCTTTAGGTAAGCTGAAGAGAGAGGTCGAAAAGGCCAAGCGTACTTTGTCCTCC |
| | | CAGATGACTACCAGAATTGAGATTGACTCTTTCGTCGACGGTATCGACTTCTCTGAGCA |
| | | ACTGTCTAGAGCTAAGTTTGAGGAGATCAACATTGAATTATTCAAGAAAACACTGAAACC |
| | | AGTTGAACAAGTCCTCAAAGACGCTGGTGTCAAGAAATCTGAAATTGATGACATTGTCT |
| | | TGGTTGGTGGTTCTACCAGAATTCCAAAGGTTCAACAATTATTGGAGGATTACTTTGAC |
| | | GGAAAGAAGGCTTCTAAGGGAATTAACCCAGATGAAGCTGTCGCATACGGTGCTGCTG |
| | | TTCAGGCTGGTGTTTTGTCTGGTGAGGAAGGTGTCGATGACATCGTCTTGCTTGATGTG |
| | | AACCCCCTAACTCTGGGTATCGAGACTACTGGTGGCGTTATGACTACCTTAATCAACAG |
| | | AAACACTGCTATCCCAACTAAGAAATCTCAAATTTTCTCCACTGCTGCTGACAACCAGCC |
| | | AACTGTGTTGATTCAAGTTTATGAGGGTGAGAGAGCCTTGGCTAAGGACAACAACTTGC |
| | | TTGGTAAATTCGAGCTGACTGGTATTCCACCAGCTCCAAGAGGTACTCCTCAAGTTGAG |
| | | GTTACTTTTGTTTTAGACGCTAACGGAATTTTGAAGGTGTCTGCCACCGATAAGGGAAC |
| | | TGGAAAATCCGAGTCCATCACCATCAACAATGATCGTGGTAGATTGTCCAAGGAGGAG |
| | | GTTGACCGTATGGTTGAAGAGGCCGAGAAGTACGCCGCTGAGGATGCTGCACTAAGAG |
| | | AAAAGATTGAGGCTAGAAACGCTCTGGAGAACTACGCTCATTCCCTTAGGAACCAAGTT |
| | | ACTGATGACTCTGAAACCGGGCTTGGTTCTAAATTGGACGAGGACGACAAAGAGACATT |
| | | GACAGATGCCATCAAAGATACCCTAGAGTTCTTGGAAGATAACTTCGACACCGCAACCA |
| | | AGGAAGAATTAGACGAACAAAGAGAAAAGCTTTCCAAGATTGCTTACCCAATCACTTCT |
| | | AAGCTATACGGTGCTCCAGAGGGTGGTACTCCACCTGGTGGTCAAGGTTTTGACGATG |
| | | ATGATGGAGACTTTGACTACGACTATGACTATGATCATGATGAGTTGTAAGCTTGGAGA |
| | | CCAATGAC (SEQ ID NO: 106) |
| PP7435_ | KAR2 | 5′-GATCTAGGTCTCCCATGCTGTCGTTAAAACCATCT-3′ |
| Chr2- | | (SEQ ID NO: 107) |
| 1167 | | 5′-GTCATTGGTCTCCAAGCTTACAACTCATCATGATCATAGTCATAG-3′ |
| | | (SEQ ID NO: 108) |
| PP7435_ | HAC1(i) | GATCTAGGTCTCACATGCCCGTAGATTCTTCTCATAAGACAGCTAGCCCACTTCCACCT |
| Chr1- | | CGTAAAAGAGCAAAGACGGAAGAAGAAAAGGAGCAGCGTCGAGTGGAACGTATCCTAC |
| 0700 | | GTAATAGGAGAGCGGCCCATGCTTCCAGAGAGAAGAAACGTAGACACGTTGAATTTCT |
| | | GGAAAACCACGTCGTCGACCTGGAATCTGCACTTCAAGAATCAGCCAAAGCCACTAAC |
| | | AAGTTGAAAGAAATACAAGATATCATTGTTTCAAGGTTGGAAGCCTTAGGTGGTACCGT |
| | | CTCAGATTTGGATTTAACAGTTCCGGAAGTCGATTTTCCCAAATCTTCTGATTTGGAACC |
| | | CATGTCTGATCTCTCAACTTCTTCGAAATCGGAGAAAGCATCTACATCCACTCGCAGAT |
| | | CTTTGACTGAGGATCTGGACGAAGATGACGTCGCTGAATATGACGACGAAGAAGAGGA |
| | | CGAAGAGTTACCCAGGAAAATGAAAGTCTTAAACGACAAAAACAAGAGCACATCTATCA |
| | | AGCAGGAGAAGTTGAATGAACTTCCATCTCCTTTGTCATCCGATTTTTCAGACGTAGAT |
| | | GAAGAAAAGTCAACTCTCACACATTTAAAGTTGCAACAGCAACAACAACAACCAGTAGA |
| | | CAATTATGTTTCTACTCCTTTGAGTCTGCCGGAGGATTCAGTTGATTTTATTAACCCAGG |
| | | TAACTTAAAAATAGAGTCCGATGAGAACTTCTTGTTGAGTTCAAATACTTTACAAATAAAA |
| | | CACGAAAATGACACCGACTACATTACTACAGCTCCATCAGGTTCCATCAATGATTTTTTT |
| | | AATTCTTATGACATTAGCGAGTCGAATCGGTTGCATCATCCAGCAGCACCATTTACCGC |
| | | TAATGCATTTGATTTAAATGACTTTGTATTCTTCCAGGAATAGTAGGCTTCGAGACCAAT |
| | | GAC (SEQ ID NO: 109) |
| PP7435_ | HAC1(i) | 5′-GATCTAGGTCTCACATGCCCGTAGATTCTTCTC-3′ |
| Chr1- | | (SEQ ID NO: 110) |
| 0700 | | 5′-GTCATTGGTCTCGAAGCCTACTATTCCTGGAAGAATACAAAG-3′ |
| | | (SEQ ID NO: 111) |
| | HAC1(i) | ATGCCAGTTGATAGTTCGCACAAGACTGCTTCTCCACTGCCACCTAG |
| | optimized | AAAGAGAGCTAAGACTGAGGAGGAAAAGGAGCAACGTAGAGTCGAG |
| | | AGAATCCTGAGAAACCGTAGAGCCGCTCACGCCTCTAGAGAGAAAA |
| | | AGAGAAGGCATGTTGAATTTCTTGAAAACCACGTCGTCGATCTCGAA |
| | | TCTGCCCTTCAAGAGTCAGCTAAAGCTACCAACAAGCTAAAGGAAAT |
| | | TCAAGACATTATCGTATCTAGACTGGAGGCACTTGGTGGTACTGTTT |
| | | CTGACCTGGATCTTACAGTTCCAGAAGTTGACTTCCCAAAATCCAGT |
| | | GATCTAGAACCTATGTCTGATCTATCTACCTCAAGCAAGTCTGAGAA |
| | | GGCAAGCACGTCAACCAGACGTTCCCTAACTGAGGACCTGGACGAA |
| | | GATGATGTCGCTGAATACGATGACGAGGAGGAGGATGAGGAACTGC |
| | | CTAGAAAAATGAAGGTTCTTAACGACAAAAACAAGTCTACCTCTATCA |
| | | AACAGGAAAAGCTCAACGAACTCCCATCCCCTCTCTCTTCCGACTTC |
| | | TCCGACGTGGACGAGGAAAAGTCTACTTTGACCCACCTGAAGTTGCA |
| | | ACAACAACAGCAACAACCTGTTGACAACTATGTCTCCACTCCTCTCT |
| | | CACTCCCAGAGGACTCGGTTGACTTCATCAACCCCGGTAACCTTAAG |
| | | ATTGAATCTGACGAGAACTTCCTTCTATCCTCTAATACCTTACAGATT |
| | | AAGCATGAAAATGATACTGACTACATTACTACCGCTCCATCCGGATC |
| | | TATCAATGACTTCTTCAATTCTTACGACATTTCTGAGTCCAACAGATT |
| | | GCACCACCCAGCTGCACCTTTTACAGCCAACGCTTTTGACCTAAACG |
| | | ACTTCGTGTTTTTCCAGGAGTAATAG (SEQ ID NO: 112) |
| PP7435_ | LHS1 | GATCTAGGTCTCCCATGAGAACACAAAAGATAGTAACAGTACTTTGTTTGCTACTAAATA |
| Chr1- | | CTGTGCTTGGAGCTCTGTTGGGCATCGATTATGGTCAAGAGTTTACTAAGGCTGTCCTA |
| 0059 | | GTGGCTCCTGGTGTCCCTTTTGAAGTTATCTTGACTCCAGACTCCAAACGTAAAGATAA |
| | | TTCAATGATGGCCATCAAGGAAAATTCCAAAGGTGAAATTGAGAGATATTATGGATCCT |
| | | CAGCTAGTTCTGTTTGTATCAGAAACCCTGAAACTTGCTTGAATCATCTGAAGTCATTGA |
| | | TAGGTGTTTCAATTGATGACGTTTCAACTATAGATTACAAGAAGTACCATTCAGGTGCTG |
| | | AGATGGTTCCATCCAAAAATAACAGGAACACGGTTGCCTTTAAGTTGGGCTCTTCTGTA |
| | | TATCCTGTAGAAGAGATACTTGCTATGAGTTTAGATGACATTAAATCTAGAGCTGAAGAT |
| | | CATTTAAAACACGCGGTGCCAGGTTCCTATTCAGTTATCAGTGATGCTGTCATCACAGT |
| | | ACCCACTTTTTTTACCCAATCGCAAAGACTGGCCTTGAAAGATGCTGCCGAAATTAGTG |
| | | GCTTAAAAGTCGTTGGCTTGGTTGATGACGGTATATCTGTGGCCGTTAACTATGCCTCT |
| | | TCAAGGCAGTTCAATGGAGACAAACAATATCATATGATCTATGACATGGGGGCTGGTTC |
| | | TTTACAGGCGACTTTGGTTTCTATATCTTCCAGTGATGATGGTGGAATTGTTATTGATGT |
| | | AGAGGCTATTGCCTATGACAAGTCGCTGGGAGGCCAGTTGTTCACACAATCTGTTTATG |
| | | ACATCCTTTTGCAGAAGTTCTTGTCTGAGCATCCTTCCTTTAGCGAGTCCGACTTCAACA |
| | | AGAATAGTAAATCTATGTCAAAACTTTGGCAAGCGGCTGAAAAGGCAAAGACAATTTTG |
| | | AGTGCAAACACTGACACAAGAGTTTCCGTTGAATCCTTATACAATGACATTGACTTTAGA |
| | | GCCACAATAGCAAGAGACGAATTCGAAGATTACAATGCAGAGCATGTTCATAGGATCAC |
| | | TGCTCCTATCATCGAGGCCTTAAGTCATCCATTGAATGGGAATCTGACGTCACCTTTTC |
| | | CACTGACCAGTTTAAGTTCAGTAATTCTCACAGGCGGGTCAACAAGAGTGCCGATGGT |
| | | GAAAAAGCACCTAGAATCTTTGCTAGGATCTGAATTGATTGCAAAGAATGTTAACGCTG |
| | | ATGAGTCAGCCGTTTTTGGTTCTACTCTCCGTGGTGTAACTTTATCGCAAATGTTCAAAG |
| | | CGAAACAGATGACCGTAAATGAAAGAAGTGTATATGACTATTGCCTAAAAGTTGGTTCTT |
| | | CAGAGATAAACGTGTTCCCAGTTGGCACCCCTCTTGCTACTAAGAAAGTGGTCGAGCT |
| | | GGAAAATGTAGACAGTGAGAACCAGCTCACGATTGGGCTCTACGAGAACGGACAATTG |
| | | TTTGCCAGTCATGAGGTTACAGACCTCAAGAAGAGTATCAAATCTCTAACTCAAGAAGG |
| | | TAAAGAGTGTTCTAATATTAATTACGAGGCTACAGTCGAGTTATCTGAGAGCAGATTGCT |
| | | TTCTTTAACTCGTCTGCAGGCCAAATGTGCTGACGAGGCTGAATATTTACCTCCTGTGG |
| | | ACACAGAGTCTGAGGATACTAAATCTGAAAACTCAACTACTAGTGAGACTATTGAAAAAC |
| | | CAAACAAGAAGCTATTCTATCCTGTGACTATACCTACTCAACTGAAATCCGTTCACGTGA |
| | | AACCAATGGGGTCCTCTACCAAGGTATCTTCATCTTTGAAAATCAAGGAGTTGAACAAG |
| | | AAGGATGCTGTAAAGAGATCGATCGAAGAATTGAAGAATCAGCTGGAATCGAAATTATA |
| | | CCGCGTGCGCTCGTATTTAGAGGATGAGGAAGTGGTTGAAAAAGGGCCAGCATCACAA |
| | | GTTGAGGCTTTGTCAACACTGGTTGCTGAGAATCTTGAGTGGTTGGACTATGATAGCGA |
| | | CGATGCATCAGCAAAAGATATCAGGGAAAAACTAAATTCTGTGTCAGATAGTGTTGCCT |
| | | TCATCAAGAGCTACATTGATCTGAACGATGTCACTTTTGATAATAATCTTTTCACTACGAT |
| | | TTACAACACTACTTTAAACTCCATGCAAAATGTTCAAGAACTAATGTTAAACATGAGTGA |
| | | GGATGCTCTGAGTTTAATGCAGCAGTATGAGAAGGAAGGTTTAGACTTCGCCAAAGAAA |
| | | GTCAAAAGATCAAAATAAAATCTCCTCCTTTATCAGACAAAGAGCTTGATAATCTCTTTAA |
| | | CACTGTTACCGAAAAGTTAGAGCATGTCAGAATGTTGACTGAAAAGGACACTATAAGTG |
| | | ATTTGCCTAGAGAGGAGCTTTTTAAGCTGTATCAAGAATTGCAGAACTACTCTTCCCGAT |
| | | TTGAAGCAATCATGGCCAGTTTGGAAGATGTACACTCTCAAAGAATCAACCGTTTGACA |
| | | GACAAGTTACGCAAACATATTGAAAGGGTGAGCAATGAAGCATTGAAGGCAGCTCTCAA |
| | | GGAAGCTAAACGTCAACAAGAGGAGGAAAAAAGCCACGAGCAGAATGAGGGAGAAGA |
| | | GCAAAGTTCTGCTTCCACTTCTCACACTAATGAAGATATAGAGGAACCATCAGAATCGC |
| | | CTAAGGTTCAAACATCCCATGATGAGTTGTAAGCTTGGAGACCAATGAC |
| | | (SEQ ID NO: 113) |
| PP7435_ | LHS1 | 5′-GATCTAGGTCTCCCATGAGAACACAAAAGATAGTAACAGTAC-3′ |
| Chr1- | | (SEQ ID NO: 114) |
| 0059 | | 5′-GTCATTGGTCTCCAAGCTTACAACTCATCATGGGATGTTT-3′ |
| | | (SEQ ID NO: 115) |
| PP7435_ | SIL1 | GATCTAGGTCTCCCATGAAAGTGACATTATCTGTGTTAGCTATTGCCTCCCAATTGGTTA |
| Chr1- | | GAATCGTTTGTTCGGAAGGAGAAAATATCTGCATAGGTGACCAGTGCTATCCGAAGAAT |
| 0550 | | TTTGAACCTGACAAGGAGTGGAAACCTGTTCAGGAAGGCCAGATTATCCCTCCAGGAT |
| | | CACACGTAAGAATGGACTTTAATACACACCAGAGAGAGGCAAAACTGGTGGAAGAGAA |
| | | TGAGGATATAGACCCCTCATCATTGGGAGTGGCTGTAGTGGATTCCACCGGTTCGTTTG |
| | | CTGATGATCAATCTTTGGAAAAGATTGAGGGACTTTCCATGGAACAACTAGATGAGAAG |
| | | TTAGAAGAACTGATTGAGCTTTCCCATGACTACGAGTACGGATCAGACATAATCTTGAG |
| | | TGATCAGTATATTTTTGGAGTAGCCGGGCTAGTTCCTACTAAGACAAAGTTTACTTCTGA |
| | | GTTGAAGGAAAAGGCCTTGAGAATTGTCGGATCATGCTTGAGAAACAATGCCGATGCG |
| | | GTAGAGAAACTACTGGGAACTGTTCCAAATACTATAACCATACAATTCATGTCAAACCTA |
| | | GTGGGTAAAGTAAATTCCACTGGAGAGAATGTTGACTCTGTTGAACAGAAACGAATCCT |
| | | TTCAATTATTGGAGCTGTTATTCCTTTCAAAATTGGAAAGGTATTGTTTGAAGCTTGTTC |
| | | GGGAACGCAGAAGCTATTACTATCCTTGGATAAACTGGAAAGTTCAGTTCAACTGAGAG |
| | | GATACCAAATGTTGGACGACTTCATTCATCACCCTGAAGAGGAACTTCTCTCTTCATTGA |
| | | CAGCAAAGGAACGATTAGTAAAGCATATTGAGTTGATTCAATCATTTTTTGCATCAGGAA |
| | | AGCATTCTCTTGATATAGCAATAAATCGTGAGTTATTCACTAGGCTGATTGCCTTACGAA |
| | | CCAATTTAGAATCTGCCAATCCAAATCTATGTAAACCATCAACTGACTTTTTGAACTGGC |
| | | TGATCGACGAAATTGAAGCTACGAAAGATACCGATCCACACTTTTCAAAAGAGCTTAAA |
| | | CATTTACGTTTTGAACTTTTTGGGAACCCATTGGCATCTAGGAAAGGTTTCTCCGATGAG |
| | | TTATAAGCTTGGAGACCAATGAC (SEQ ID NO: 116) |
| PP7435_ | SIL1 | 5′-GATCTAGGTCTCCCATGAAAGTGACATTATCTGTGTTAGC-3′ |
| Chr1- | | (SEQ ID NO: 117) |
| 0550 | | 5′-GTCATTGGTCTCCAAGCTTATAACTCATCGGAGAAACCTTTC-3′ |
| | | (SEQ ID NO: 118) |
| PP7435_ | ERJ5 | GATCTAGGTCTCCCATGAAACTACACCTTGTGATTCTCTGTTTGATCACTGCTGTCTACT |
| Chr1- | | GTTTCAGTGCTGTTGACAGAGAAATCTTTCAGCTCAACCATGAATTACGCCAGGAATAC |
| 0136 | | GGAGATAATTTTAATTTCTATGAATGGTTGAAGCTTCCAAAAGGTCCCTCGTCCACGTTT |
| | | GAAGATATCGACAACGCGTACAAGAAACTATCCCGTAAGTTACACCCCGATAAGATAAG |
| | | ACAGAAGAAACTATCCCAGGAACAATTTGAGCAATTGAAGAAAAAGGCTACCGAAAGAT |
| | | ACCAACAATTGAGTGCTGTGGGATCCATCTTAAGATCCGAGAGCAAAGAGCGTTACGAT |
| | | TATTTTGTCAAACATGGATTCCCAGTCTATAAAGGTAACGATTACACCTATGCCAAGTTT |
| | | AGACCATCCGTTTTGCTCACAATTTTCATCCTTTTTGCGTTAGCTACGTTAACCCACTTT |
| | | GTCTTTATCAGATTGTCGGCCGTGCAATCTAGAAAAAGACTGAGTTCGTTGATAGAGGA |
| | | GAACAAACAGCTGGCTTGGCCACAAGGTGTTCAAGATGTCACTCAAGTGAAGGACGTC |
| | | AAAGTCTATAACGAACATCTACGTAAATGGTTTTTGGTATGTTTCGACGGATCCGTTCAT |
| | | TATGTGGAGAACGATAAAACCTTCCATGTTGATCCGGAAGAAGTTGAACTCCCATCTTG |
| | | GCAGGACACTCTTCCAGGTAAATTAATAGTCAAGCTGATACCCCAGCTTGCTAGAAAGC |
| | | CACGATCTCCAAAGGAGATCAAGAAGGAAAATTTAGATGATAAAACCAGAAAGACAAAA |
| | | AAACCTACAGGGGATTCCAAAACTTTACCTAACGGTAAAACCATTTATAAAGCTACCAAA |
| | | TCCGGTGGACGTAGAAGGAAATAAGCTTGGAGACCAATGAC (SEQ ID NO: 119) |
| PP7435_ | ERJ5 | 5′-GATCTAGGTCTCCCATGAAACTACACCTTGTGATTCTC-3′ |
| Chr1- | | (SEQ ID NO: 120) |
| 0136 | | 5′-GTCATTGGTCTCCAAGCTTATTTCCTTCTACGTCCACC-3′ |
| | | (SEQ ID NO: 121) |

b) Creating the Native and Synthetic MSN4 Overexpression Strains

One silent mutation was introduced into the native coding sequence of P. pastoris MSN4 to remove a BsaI restriction site. This coding sequence was introduced into BB1 of the GoldenPiCS system. The synthetic MSN4 coding sequence was assembled by fusing a transcription activator domain (VP64) and a nuclear localization (SV40) sequence with MSN4's native DNA binding domain from nucleotide no. 883 to 1071. The DNA binding domain was identified by sequence homology to the published amino acid sequence in Nicholls et al. 2004 (Eukaryot Cell. doi: 10.1128/EC.3.5.1111-1123.2004). This synthetic coding sequence (synMSN4) was introduced into BB1 of the GoldenPiCS system. S. cerevisiae MSN2, S. cerevisiae MSN4, A. niger MSN4 homolog Seb1 and the Y. lipolytica MSN4 homolog were amplified from genomic DNA of S. cerevisiae CEN.PK, A. niger CBS513.88 and Y. lipolytica DSMZ, respectively and introduced into BB1.

Each MSN4 coding sequence was combined with the glyceraldehyde-3-phosphate dehydrogenase (GAP) promoter and the S. cerevisiae CYC1 transcription terminator into the integration plasmid BB3rN (e.g. for native P. pastoris MSN4 189_BB3rN or 142_BB3eH). P. pastoris MSN4 was also combined with the THI11 promoter and the IDP1 terminator (253_BB3eH), or the PORI promoter and the IDP1 terminator (254_BB3eH). The synMSN4 coding sequence was additionally combined with the TH111 promoter (Landes et al. 2016. Biotechnol Bioeng. doi: 10.1002/bit.26041) and the IDP1 transcription terminator (258_BB3eH) or the SBH17 promoter and the TDH3 terminator (191_BB3aK). The synMSN4 coding sequence was also combined with the GAP promoter and the TDH3 transcription terminator into the integration plasmid 208_BB3aK. All integration plasmids were linearized with the restriction enzyme AscI prior to their application for transforming the basic production strains. Titer and yield (titer per wet cell weight) of the clones overexpressing MSN4 or syntheticMSN4 were determined in small scale screenings and compared to their parental basic production strains (Example 3).

c) Creating the (Synthetic)MSN4+KAR2 Overexpression Strains

An overexpression cassette only containing KAR2 was assembled in the integration plasmid BB3eH (219_BB3eH). This plasmid derives from combining the BB1 plasmids with the KAR2 coding sequence and the GAP promoter as well as the RPS3 terminator.

The best clones overexpressing MSN4 or syntheticMSN4 in terms of product yield determined in small scale screenings (Example 3) were chosen after transformation with the respective plasmid of Example 2b and further transformed with the SmaI linearized KAR2 integration plasmid 219_BB3eH. This finally yielded clones with two different overexpression cassettes introduced by two sequential transformations with two different integration plasmids.

d) Creating the (Synthetic)MSN4+HAC1(i) Overexpression Strains

The induced (i) version of the HAC1(i) coding sequence was created by removing the alternative intron from nucleotide no. 857 to 1178 according to Guerfal et al. 2010 (Microb Cell Fact. doi: 10.1186/1475-2859-9-49). The coding sequence was introduced into BB1. Additionally a codon-optimized HAC1(i) sequence was used for overexpression of Hac1(i). It was further combined with the promoter of FDH1 and the terminator of RPL2A in a BB2 plasmid. Other BB2 constructs contained HAC1 under control of the MDH3 promoter and the RPL2A terminator, or the ADH2 promoter and the RPL2A terminator.

The integration plasmids 243_BB3eH, 253_BB3eH, 254_BB3eH and 257_BB3eH carrying the MSN4+HAC1(i) combination under control of different promoters were created by combining the BB2s of Example 2d with a BB2 plasmid containing an expression cassette for, MSN4 (Example 2b). The same combination was also generated by the sequential transformation with the integration plasmid BB3rN only carrying MSN4 (189_BB3rN) and the integration plasmid BB3eH only carrying HAC1(i) with the FDH1 promoter and the RPL2A terminator (234_BB3eH). For the plasmid carrying the combination synMSN4+HAC1(i) in an integration plasmid (258_BB3eH), the BB2 of Example 2d was combined with a BB2 plasmid, which derived from the BB1 plasmid with synMSN4 (Example 2b) combined with the TH111 promoter and the IDP1 transcription terminator. Both integration plasmids were linearized with the restriction enzyme SmaI prior to their application for transforming the basic production strains.

e) Creating the (Synthetic)MSN4+KAR2 and/or LHS1, (Synthetic)MSN4+KAR2 and/or SIL1 (Synthetic)MSN4+KAR2+LHS1 or SIL1 and ERJ5 Overexpression Strains

The coding sequences of KAR2 (7 silent mutations required), LHS1 (1 silent mutation required), SIL1 (no mutations) and ERJ5 (1 silent mutations required) were introduced into BB1 of the GoldenPiCS system. The integration plasmid 219_BB3eH contains KAR2 with the GAP promoter and the RPS3 transcription terminator. The overexpression of KAR2 in combination with LHS1 was assembled in the integration plasmid 174_BB3eH, which derives from two BB2s; one containing KAR2 with the GAP promoter and the RPS3 transcription terminator and the other BB2 containing LHS1 with the PORI promoter and the IDP1 transcription terminator. The overexpression of KAR2 in combination with SIL1 was assembled in the integration plasmid 078_BB3eH, which derives from two BB2s; one containing KAR2 with the GAP promoter and the RPS3 transcription terminator and the other BB2 containing SIL1 with the PORI promoter and the IDP1 transcription terminator. The overexpression of KAR2 in combination with LHS1 and ERJ5 was assembled in the integration plasmid 052_BB3eH, which derives from three BB2s; the first containing KAR2 with the GAP promoter and the S. cerevisiae CYC1 transcription terminator, the second BB2 containing LHS1 with the PORI promoter and the IDP1 transcription terminator and the third BB2 containing ERJ5 with the MDH3 promoter and the TDH1 transcription terminator.

The best clones in terms of yield (titer per biomass) determined in small scale screenings (Example 3) were chosen after transformation with the respective plasmid of Example 2b and further transformed with the respective SmaI linearized BB3eH integration plasmid mentioned above. This finally yielded clones with two different overexpression cassettes introduced by two sequential transformations with two different integration plasmids.

### Example 3: Screening for Increased scFv or vHH Secretion

In small-scale screenings, up to 20 transformants of each overexpression combination were tested after transformation. Transformants were evaluated by comparing their scFv or vHH titer in the supernatant, their wet cell weight (biomass after centrifugation and supernatant removal) and their scFv or vHH yield (titer per wet cell weight) to those of the respective parental basic production strain. For each overexpression combination an average fold-change of titer, yield and wet cell weight was determined to assess the secretion improvement. The average fold-change of titer, yield and wet cell weight was calculated by dividing the arithmetic mean of titer, yield and wet cell weight of all transformants by the arithmetic mean of titer, yield and wet cell weight of the four biological replicates of the basic production strains cultivated on the same deep well plate.

a) Small Scale Screening Cultivations of scFv or vHH Production Strains

2 mL YP-medium (10 g/L yeast extract, 20 g/L peptone) containing 10 g/L glucose and 50 μg/mL Zeocin (basic production strains) or 50 μg/mL Zeocin and 500 μg/mL G418 and/or 200 μg/mL Hygromycin and/or 100 μg/mL Nourseothricin (depending on the integration plasmids of the engineered strains) were inoculated with a single colony of a P. pastoris clone and grown overnight at 25° C. These cultures were transferred to 2 mL of synthetic screening medium M2 or ASMv6 (media compositions are given below) supplemented with a glucose feed tablet (Kuhner, Switzerland; CAT #SMFB63319) or x % of enzyme (m2p media development kit) and incubated for 1 to 25 h at 25° C. at 280 rpm in 24 deep well plates. Aliquots of these cultures (corresponding to a final OD₆₀₀ of 4 or 8) were transferred into 2 mL of synthetic screening medium M2 or ASMv6 (in the case of ASMv6 with the m2p media development kit in fresh 24 deep well plates. 0.5 vol % of pure methanol were added initially and 1 vol % of pure methanol were repeatedly added after 19 hours, 27 hours, and 43 hours. After 48 hours, the cells were harvested by centrifugation at 2,500×g for 10 min at room temperature and prepared for analysis. Biomass was determined by measuring the cell weight of 1 mL cell suspension, while determination of the recombinant secreted protein in the supernatant is described in the following Examples 3b-3c.

Synthetic screening medium M2 contained per liter: 22.0 g Citric acid monohydrate 3.15 g (NH₄)₂HPO₄, 0.49 g MgSO₄*7H₂O, 0.80 g KCl, 0.0268 g CaCl₂*2H₂O, 1.47 mL PTM1 trace metals, 4 mg Biotin; pH was set to 5 with KOH (solid)

Synthetic screening medium ASMv6 contained per liter: 44.0 g Citric acid monohydrate, 12.60 g (NH₄)₂HPO₄, 0.98 g MgSO₄*7H₂O, 5.28 g KCl, 0.1070 g CaCl₂*2H₂O, 2.94 mL PTM1 trace metals, 8 mg Biotin; pH was set to 6.5 with KOH (solid)

b) SDS-PAGE & Western Blot Analysis

For protein gel analysis the NuPAGE® Novex® Bis-Tris system was used, using 12% Bis-Tris gels with MOPS running buffer or 4-12% Bis-Tris gels with MES running buffer (all from Invitrogen). After electrophoresis, the proteins were either visualized by colloidal Coomassie staining or transferred to a nitrocellulose membrane for Western blot analysis. Therefore, the proteins were electroblotted onto a nitrocellulose membrane using the Biorad Trans-Blot® Turbo™ Transfer System with ready-to-use membranes and filter papers and the program Turbo for minigels (7 min). After blocking, the Western Blots were probed with the following antibodies: The His-tagged scFv and vHH were detected with the following antibody: Anti-polyHistidin-Peroxidase antibody (A7058, Sigma), diluted 1:2,000. Detection was performed with the chemiluminescent Super Signal West Chemiluminescent Substrate (Thermo Scientific) for HRP-conjugates.

c) Quantification by Microfluidic Capillary Electrophoresis (mCE)

The ‘LabChip GX/GXII System’ (PerkinElmer) was used for quantitative analysis of secreted protein titer in culture supernatants. The consumables ‘Protein Express Lab Chip’ (760499, PerkinElmer) and ‘Protein Express Reagent Kit’ (CLS960008, PerkinElmer) were used. Briefly, several μL of all culture supernatants are fluorescently labeled and analyzed according to protein size, using an electrophoretic system based on microfluidics. Internal standards enable approximate allocations to size in kDa and approximate concentrations of detected signals.

### Example 4: Fed Batch Cultivations

Clones of the engineered strains (Example 2) were selected after small scale screening cultivations (Example 3). The selected clones were further evaluated in larger cultivation volumes by fed batch bioreactor cultivations. Secretion improvements in small scale screenings, which were also present in fed batch bioreactor cultivations, were verified.

a) Procedure of Fed Batch Bioreactor Cultivations

Respective strains were inoculated into wide-necked, baffled, covered 300 mL shake flasks filled with 50 mL of YPhyG and shaken at 110 rpm at 28° C. over-night (pre-culture 1). Pre-culture 2 (100 mL YPhyG in a 1000 mL wide-necked, baffled, covered shake flask) was inoculated from pre-culture 1 in a way that the OD₆₀₀ (optical density measured at 600 nm) reached approximately 20 (measured against YPhyG media) in late afternoon (doubling time: approximately 2 hours). Incubation of pre-culture 2 was performed at 110 rpm at 28° C., as well.

The fed batches were carried out in 0.8 L working volume bioreactor (Minifors, Infors, Switzerland). All bioreactors (filled with 400 mL BSM-media with a pH of approximately 5.5) were individually inoculated from pre-culture 2 to an OD600 of 2.0. Generally, P. pastoris was grown on glycerol to produce biomass and the culture was subsequently subjected to glycerol feeding followed by methanol feeding.

In the initial batch phase, the temperature was set to 28° C. Over the period of the last hour before initiating the production phase it was decreased to 24° C. and kept at this level throughout the remaining process, while the pH dropped to 5.0 and was kept at this level. Oxygen saturation was set to 30% throughout the whole process (cascade control: stirrer, flow, oxygen supplementation). Stirring was applied between 700 and 1200 rpm and a flow range (air) of 1.0-2.0 L/min was chosen. Control of pH at 5.0 was achieved using 25% ammonium. Foaming was controlled by addition of antifoam agent Glanapon 2000 on demand.

During the batch phase, biomass was generated (μ˜0.30/h) up to a wet cell weight (WCW) of approximately 110-120 g/L. The classical batch phase (biomass generation) would last about 14 hours. Glycerol was fed with a rate defined by the equation 2.6+0.3*t (g/h), so a total of 30 g glycerol (60%) was supplemented within 8 hours. The first sampling point was selected to be 20 hours (0 h induction time).

In the following 18 hours (from process time 20 to 38 hours), a mixed feed of glycerol/methanol was applied: glycerol feed rate defined by the equation: 2.5+0.13*t (g/h), supplying 66 g glycerol (60%) and methanol feed rate defined by the equation: 0.72+0.05*t (g/h), adding 21 g of methanol.

During the next 72-74 hours (from process time 38 to 110-112 hours) methanol was fed with a feed rate defined by the equation 2.2+0.016*t (g/L)).

YPhyG preculture medium (per liter) contained: 20 g Phytone-Peptone, 10 g Bacto-Yeast Extract, 20 g glycerol

Batch medium: Modified Basal salt medium (BSM) (per liter) contained: 13.5 mL H₃PO₄ (85%), 0.5 g CaCl.2H₂O, 7.5 g MgSO₄.7H₂O, 9 g K₂SO₄, 2 g KOH, 40 g glycerol, 0.25 g NaCl, 4.35 mL PTM1, 0.1 mL Glanapon 2000 (antifoam)

PTM1 Trace Elements (per liter) contains: 0.2 g Biotin, 6.0 g CuSO₄.5H₂O, 0.09 g KI, 3.00 g MnSO₄.H₂O, 0.2 g Na₂MoO₄.2H₂O, 0.02 g H₃BO₃, 0.5 g CoCl₂, 42.2 g ZnSO₄.7H₂O, 65.0 g FeSO₄.7H₂O, and 5.0 mL H₂SO₄ (95%-98%).

Feed-solution glycerol (per kg) contained: 600 g glycerol, 12 mL PTM1 Feed-solution methanol contained: pure methanol.

b) Sample Analysis of Fed Batch Bioreactor Cultivations

Samples were taken at various time points with the following procedure: the first 3 mL of sampled cultivation broth (with a syringe) were discarded. 1 mL of the freshly taken sample (3-5 mL) was transferred into a 1.5 mL centrifugation tube and spun for 5 minutes at 13,200 rpm (16,100 g). Supernatants were diligently transferred into a separate vial and stored at 4° C. or frozen until analysis. 1 mL of cultivation broth was centrifuged in a tared Eppendorf vial at 13,200 rpm (16,100 g) for 5 minutes and the resulting supernatant was accurately removed. The vial was weighed (accuracy 0.1 mg), and the tare of the empty vial was subtracted to obtain wet cell weights.

Supernatants of the individual sampling points of each bioreactor cultivation were analyzed using mCE (microfluidic capillary electrophoresis, GXII, Perkin-Elmer) against BSA or purified standard material (for scR-GG-6×HIS and vHH-GG-6×HIS).

### Example 5: Improvement of Recombinant Protein Production and Secretion by Overexpressions of Transcription Factor(s) and Helper Gene(s)

The secretion improvement is measured by titer and yield fold-change values that refer to the respective unengineered basic production strains (Example 1).

a) Improvement of vHH Protein Secretion Yields by Overexpression of a Transcription Factor Alone or in Combination with Helper Gene(s)—Results from Small Scale Screenings

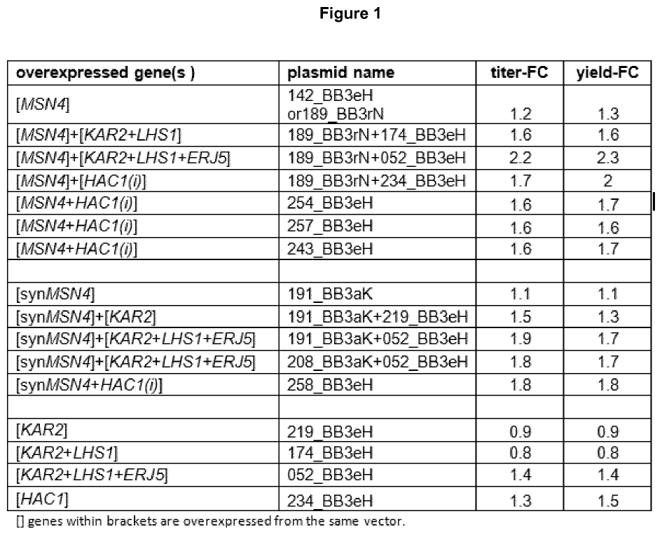

FIG. 1 lists overexpressed genes or gene combinations that increase vHH secretion in P. pastoris in small scale screening (Example 3). The fold-change values of small scale screenings are an arithmetic mean of up to 20 clones/transformants (see Example 3).

Secretion of vHH is increased by overexpression of the transcription factor Msn4 (FIG. 1). Both the native and the synthetic Msn4 variants increase vHH titers and yields to similar levels. Unexpectedly, overexpression of the chaperone Kar2 alone or in combination with the co-chaperone Lhs1 did not increase vHH secretion. Only when these are co-overexpressed with the transcription factor Msn4 or synMsn4 increased vHH titers and yields were observed. Further co-expression of a Hsp40 protein such as Erj5 led to a further increase of vHH secretion.

Also the co-expression of Msn4 or synMsn4 together with Hac1 resulted in enhanced vHH secretion, and outperformed single Hac1 overexpression. Thereby, similar levels of enhancement were obtained independently whether the two transcription factors were expressed form the same vector or from two separate vectors. Also, there was no significant difference when different promoter pairs were used for the expression of the two transcription factors.

b) Improvement of vHH Protein Secretion Yields by Overexpression of a Transcription Factor Alone or in Combination with Helper Gene(s)—Results from Fed Batch Bioreactor Cultivations

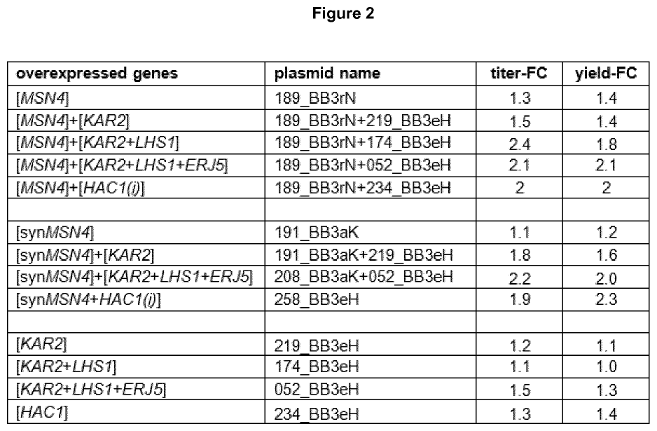

FIG. 2 lists overexpressed genes or gene combinations that increase vHH secretion in P. pastoris in fed batch cultivations (Example 4). The fold-change values of fed batch cultivations are those of the single selected clone.

The positive impact of overexpressing the transcription Msn4 on recombinant protein production observed in screenings were also confirmed controlled bioreactor cultivations (FIG. 2). As in the screenings, combined overexpression of Msn4 or synMsn4 with chaperones or other transcription factors markedly exceeded the performance of strains overexpressing just the latter factors. No obvious difference between overexpression of the native and the synthetic version of Msn4 was seen regarding the beneficial effect on vHH secretion.

c) Improvement of scFv Protein Secretion Yields by Overexpression of a Transcription Factor Alone or in Combination with Helper Gene(s)—Results from Small Scale Screenings

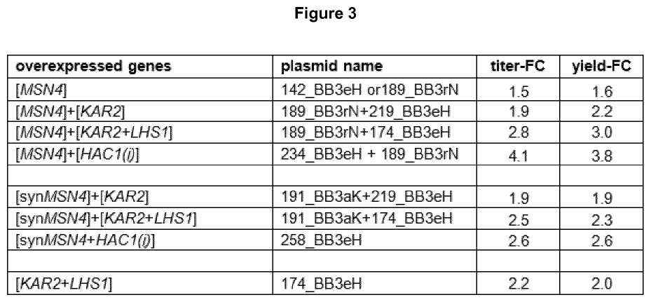

FIG. 3 lists overexpressed genes or gene combinations that increase scFv secretion in P. pastoris in small scale screening (Example 3). The fold-change values of small scale screenings are an arithmetic mean of up to 20 clones/transformants (see Example 3).

Overexpression of Msn4 also enhanced secretion levels of scFv, which represents another model POI (FIG. 3). As for vHH, secretion yields and titers were further enhanced by combining Msn4 or synMsn4 overexpression with overexpression of chaperones such as Kar2 alone or in combination with Lhs1, and exceeded the improvement obtained by Kar2 and Lhs1 overexpression without Msn4. Also the combination of Msn4 or synMsn4 with Hac1 overexpression had a positive impact on scFv secretion.

d) Improvement of scFv Protein Secretion Yields by Overexpression of a Transcription Factor Alone or in Combination with Helper Gene(s)—Results from Fed Batch Bioreactor Cultivations

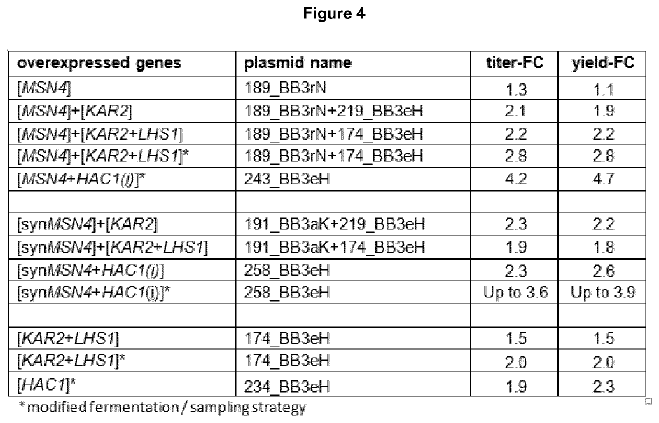

FIG. 4 lists overexpressed genes or gene combinations that increase vHH secretion in P. pastoris in fed batch cultivations (Example 4). The fold-change values of fed batch cultivations are those of the single selected clone.

Also for the second recombinant model protein, the results obtained in screenings were confirmed under controlled process-like bioreactor conditions (FIG. 4). Overexpression of Msn4 alone improved scFv titers and yields compared to the wild type production strain (parent). Co-overexpression of Msn4 with chaperones or other transcription factors such as Hac1 stimulated scFv secretion compared to overexpression of chaperones or Hac1 alone.

e) Improvement of scFv Secretion (Titer and Yield) by Overexpression of MSN2/4 Homologs from Other Species in Fed Batch Bioreactor Cultivations

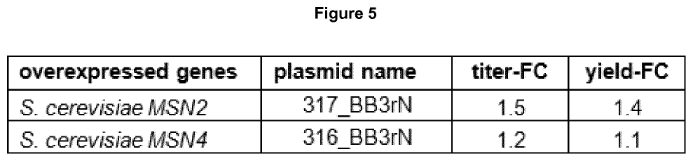

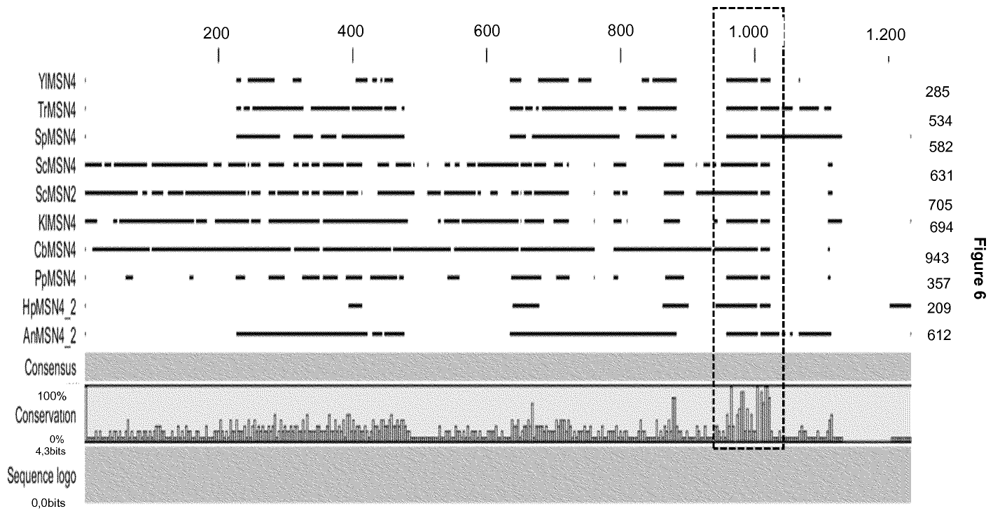

FIG. 5 lists overexpressed MSN2/4 homologs that increase scFv secretion in P. pastoris in fed batch cultivations (Example 4). The fold-change values of fed batch cultivations are those of the single selected clone.

Overexpression of the two Msn4 homologs from S. cerevisiae had a positive effect on scFv secretion (FIG. 5), which confirms that also homologs from other species have the positive effect on protein secretion in P. pastoris. Together with the results from native Msn4 P. pastoris and the synthetic Msn4 variant, this also points to the conserved effect of targeted Msn4 overexpression to improve recombinant protein production in other production hosts and underlines the versatile applicability of our approach.

### Example 6: MSN4 Alignment and Sequence Identity to PpMSN4

The MSN2/4 functional knowledge derives from Saccharomyces cerevisiae, due to it being the most important model organism for eukaryotic cells. In this context, it is important to mention that S. cerevisiae underwent a whole-genome duplication (WGD). This causes S. cerevisiae's genome to have very similar copies of many of its genes. The redundant transcription factors Msn2p and Msn4p are such a case. Due to this functional redundancy, these transcription factors are usually addressed as MSN2/4. The functional description of proteins of other yeasts are derived from experiments with the model organism S. cerevisiae. Pichia pastoris for example did not undergo a WGD and therefore only has one homolog, Msn4p. Because there is basically no functional distinction between Msn2p and Msn4p in S. cerevisiae, there cannot be a reasonable distinction of these transcription factors in other yeasts.

The alignment was performed with the software CLC Main Workbench (QIAGEN Bioinformatics) and can be viewed in the FIG. 6. The only region of strong conservation is highlighted in the dotted box in FIG. 6 and consists of the protein structural motif of the zinc finger. This is the known DNA binding domain of the well characterized transcription factor Msn4p and Msn2p in S. cerevisiae (ScMSN4/2) and can likely be used to derive the same function in other organisms (Nicholls et al. 2004).

The zinc finger in S. cerevisiae's MSN2/4 has a C₂H₂-like fold. The amino acid sequence motif is X₂-C-X_{2,4}-C-X₁₂-H-X_{3,4,5}-H, which is also depicted in FIG. 7. This motif can be clearly observed, if it is zoomed into the strongly conserved area (black dotted box of FIG. 6) of the sequence alignment (FIG. 7).

The consensus sequence of the MSN4-like C₂H₂ type zinc finger DNA binding domain is highlighted in grey. The C₂H₂ motif is marked with black asterisks (*). The consensus sequence is:

(SEQ ID NO: 87)
KPFVCTLCSKRFRRXEHLKRHXRSXHSXEKPFXCXXCXKKFSRSDNL
XQHLRTH.

Further, pairwise sequence similarities/identities between the full length Msn4p of P. pastoris and each homolog of the other organisms was investigated by a global pairwise sequence alignment with the EMBOSS Needle algorithm. Pairwise sequence similarities/identities were also investigated for the DNA-binding domain of Msn4p of P. pastoris and the DNA-binding domains of each homolog of the other organisms. The EMBOSS Needle webserver (https://www.ebi.ac.uk/Tools/psa/emboss_needle/) was used for pairwise protein sequence alignment using default settings (Matrix: BLOSUM62; Gap open:10; Gap extend: 0.5; End Gap Penalty: false; End Gap Open: 10; End Gap Extend: 0.5). EMBOSS Needle reads two input sequences and writes their optimal global sequence alignment to file. It uses the Needleman-Wunsch alignment algorithm to find the optimum alignment (including gaps) of two sequences along their entire length.

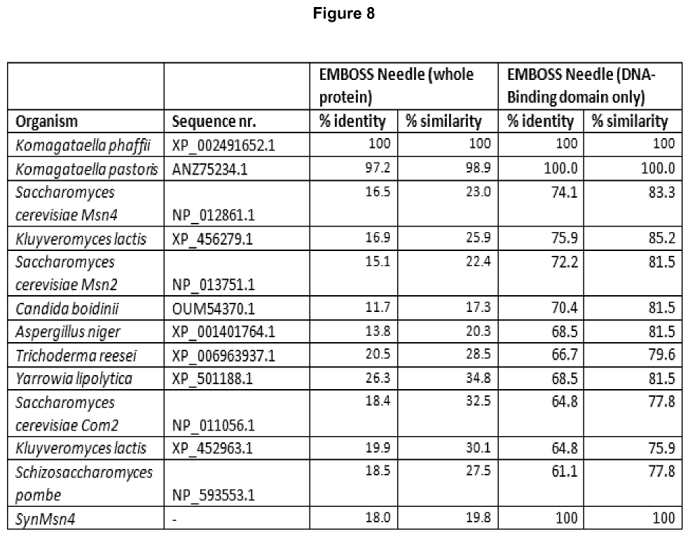

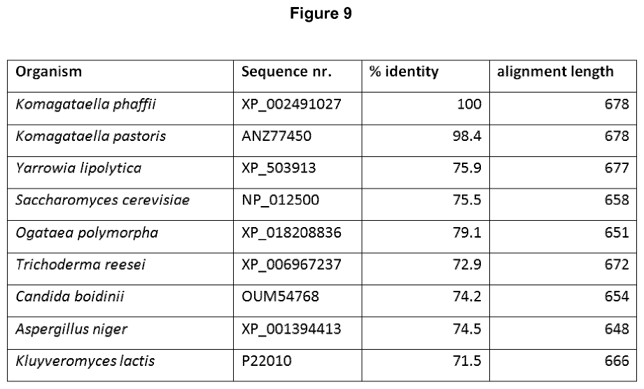

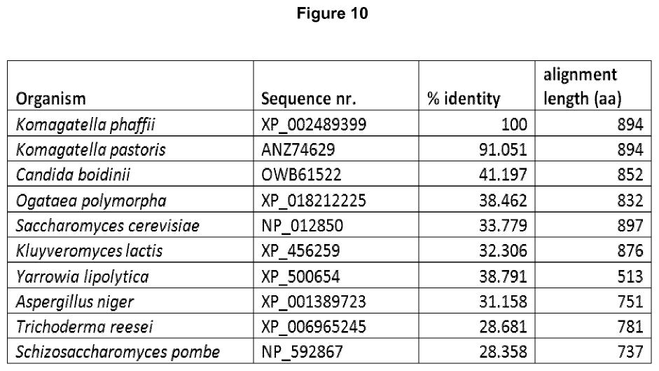

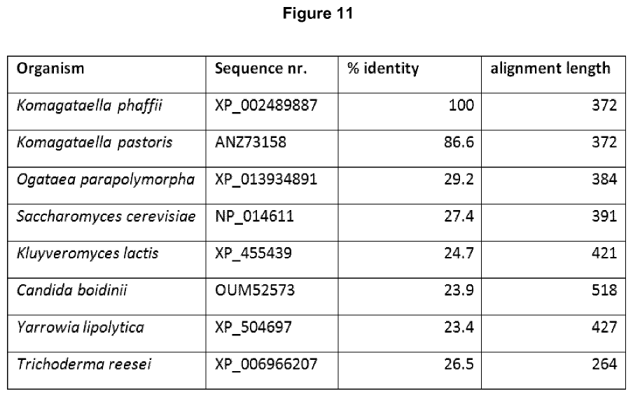

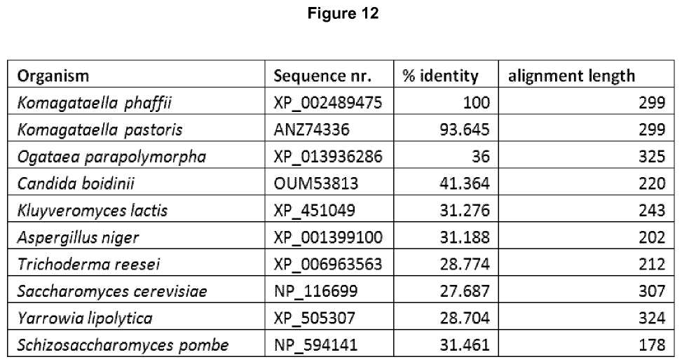

The identity results are listed in FIG. 8. As expected, the global sequence identities of the full length Msn4 show far less conservation then the DNA-binding domain only.

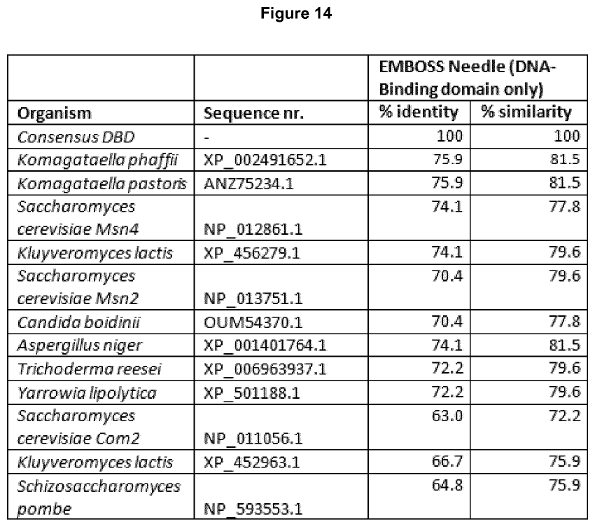

Pairwise sequence similarities/identities were investigated between the consensus sequence of the DNA-binding domain (DBD) of Msn4p/Msn2p and the DNA-binding domains of each homolog of the other organisms by the global pairwise sequence alignment with the EMBOSS Needle algorithm as well (see FIG. 14).

### Example 7: HAC1 Alignment and Sequence Similarity to PpHAC1

The alignment was performed with the software CLC Main Workbench (QIAGEN Bioinformatics).

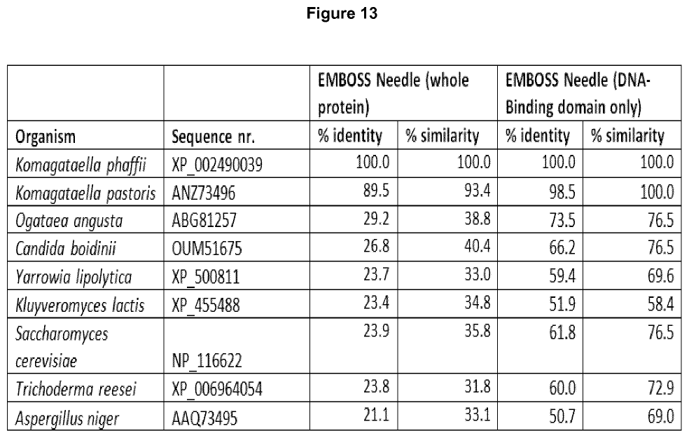

Pairwise sequence similarities/identities between the full length Hac1p of P. pastoris or its DNA-binding domain and each homolog of the other organisms was investigated. The global similarity/identity was assessed by a global pairwise sequence alignment with the EMBOSS Needle algorithm. (FIG. 13).

1. A method of increasing the yield of a recombinant protein of interest in a eukaryotic host cell, comprising overexpressing in said host cell at least one polynucleotide encoding at least one transcription factor, thereby increasing the yield of said recombinant protein of interest in comparison to a host cell which does not overexpress the polynucleotide encoding said transcription factor, wherein the transcription factor comprises at least:
a) a DNA binding domain comprising:
i) an amino acid sequence as shown in SEQ ID NO: 1, or
ii) a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87, and
b) an activation domain.

2. The method according to claim 1, comprising:
i) engineering the host cell to overexpress at least one polynucleotide encoding at least one transcription factor comprising at least:
a) a DNA binding domain comprising:
a1) an amino acid sequence as shown in SEQ ID NO: 1, or
a2) a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87, and
b) an activation domain,
ii) engineering said host cell to comprise a polynucleotide encoding the protein of interest,
iii) culturing said host cell under suitable conditions to overexpress the at least one polynucleotide encoding at least one transcription factor and to overexpress the protein of interest, optionally
iv) isolating the protein of interest from the cell culture, and optionally
v) purifying the protein of interest.

3. A method of manufacturing a recombinant protein of interest by a eukaryotic host cell comprising:
i) providing the host cell engineered to overexpress at least one polynucleotide encoding at least one transcription factor, wherein the host cell further comprises a polynucleotide encoding a protein of interest, wherein the transcription factor comprises at least:
a) a DNA binding domain comprising:
a1) an amino acid sequence as shown in SEQ ID NO: 1, or
a2) a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87,
b) an activation domain,
ii) culturing said host cell under suitable conditions to overexpress the at least one polynucleotide encoding at least one transcription factor and to overexpress the protein of interest, optionally
iii) isolating the protein of interest from the cell culture, and optionally
iv) purifying the protein of interest, and optionally
v) modifying the protein of interest, and optionally
vi) formulating the protein of interest.

4. The method according to claim 1, wherein overexpression of said transcription factor increases the yield of the model protein scFv (SEQ ID NO. 13) and/or vHH (SEQ ID NO. 14) compared to the host cell prior to engineering.

5. The method according to claim 1, wherein the polynucleotide encoding the at least one transcription factor is integrated in the genome of said host cell or contained in a vector or plasmid, which does not integrate into the genome of said host cell.

6. The method according to claim 1, wherein said polynucleotide encoding at least one transcription factor encodes for a heterologous or homologous transcription factor.

7. The method according to claim 6, wherein the overexpression of the polynucleotide encoding a heterologous transcription factor is achieved by
i) exchanging or modifying a regulatory sequence operably linked to said polynucleotide encoding the heterologous transcription factor, or
ii) introducing one or more copies of the polynucleotide encoding the heterologous transcription factor under the control of a promoter into the host cell.

8. The method according to claim 6, wherein the overexpression of the polynucleotide encoding a homologous transcription factor is achieved by
i) using a promoter which drives expression of said polynucleotide encoding the homologous transcription factor,
ii) exchanging or modifying a regulatory sequence operably linked to said polynucleotide encoding the homologous transcription factor, or
iii) introducing one or more copies of the polynucleotide encoding the homologous transcription factor under the control of a promoter into the host cell.

9. The method according to claim 1, wherein the overexpression of the polynucleotide is achieved by
i) exchanging the native promoter of said homologous transcription factor by a different promoter operably linked to the polynucleotide encoding the homologous transcription factor,
ii) exchanging the native terminator sequence of said heterologous and/or homologous transcription factor by a more efficient terminator sequence,
iii) exchanging the coding sequence of said heterologous and/or homologous transcription factor by a codon-optimized coding sequence, which codon-optimization is done according to the codon-usage of said host cell,
iv) exchanging of a native positive regulatory element of said homologous transcription factor by a more efficient regulatory element,
v) introducing another positive regulatory element, which is not present in the native expression cassette of said homologous transcription factor,
vi) deleting a negative regulatory element, which is normally present in the native expression cassette of said homologous transcription factor, or
vii) introducing one or more copies of the polynucleotide encoding a heterologous and/or homologous transcription factor, or a combination thereof.

10. The method according to any one of claims 1 to 9, wherein the transcription factor comprises an amino acid sequence as shown in SEQ ID NOs: 15-27.

11. The method according to claim 1, wherein the transcription factor additionally comprises a nuclear localization signal.

12. The method according to claim 11, wherein said nuclear localization signal is a homolog or a heterolog nuclear localization signal.

13. The method according to claim 1, wherein said transcription factor does not stimulate the promotor used for expression of the protein of interest.

14. The method of claim 1, wherein the eukaryotic host cell is a fungal host cell, preferably a yeast host cell selected from the group consisting of Pichia pastoris, Hansenula polymorpha, Trichoderma reesei, Aspergillus niger, Saccharomyces cerevisiae, Kluyveromyces lactis, Yarrowia lipolytica, Pichia methanolica, Candida boidinii, Komagataella spp. and Schizosaccharomyces pombe.

15. The method of claim 1, wherein the recombinant protein of interest is an enzyme, a therapeutic protein, a food additive or feed additive.

16. The method according to claim 15, wherein the therapeutic protein is an antigen binding protein.

17. The method according to claim 1, further comprising overexpressing in said host cell or engineering said host cell to overexpress at least one polynucleotide encoding at least one ER helper protein.

18. The method according to claim 17, wherein said ER helper protein has an amino acid sequence as shown in SEQ ID NO: 28 or a functional homolog thereof having at least 70% sequence identity to an amino acid sequence as shown in SEQ ID NO: 28.

19. The method according to claim 1, further comprising overexpressing in said host cell or engineering said host cell to overexpress at least two polynucleotides encoding at least two ER helper proteins.

20. The method according to claim 19, wherein:
a) the first ER helper protein has an amino acid sequence as shown in SEQ ID NO: 28 or a functional homologue thereof having at least 70% sequence identity to the amino acid sequence as shown in SEQ ID NO: 28, and
b) the second ER helper protein has an amino acid sequence:
i) as shown in SEQ ID NO: 37, or a functional homologue thereof having at least 25% sequence identity to the amino acid sequence as shown in SEQ ID NO: 37, or
ii) as shown in SEQ ID NO. 47, or a homologue thereof, wherein the homologue has at least 20% sequence identity to the amino acid sequence as shown in SEQ ID NO. 47 and optionally
c) the third ER helper protein has an amino acid sequence:
i) as shown in SEQ ID NO: 55, or a functional homologue thereof having at least 25% sequence identity to the amino acid sequence as shown in SEQ ID NO: 55.

21. The method according to claim 1, further comprising overexpressing in said host cell or engineering said host cell to overexpress at least one polynucleotide encoding one additional transcription factor.

22. The method according to claim 21, wherein the additional transcription factor comprises at least:
a) a DNA binding domain comprising:
i) an amino acid sequence as shown in SEQ ID NO: 65, or
ii) a functional homolog of the amino acid sequence as shown in SEQ ID NO: 65 having at least 50% sequence identity to an amino acid sequence as shown in SEQ ID NO: 65, and
b) an activation domain.

23. The method according to claim 22, wherein the additional transcription factor comprises an amino acid sequence as shown in SEQ ID NOs: 74-82.

24. The method according to claim 21, wherein said additional transcription factor does not stimulate the promotor used for expression of the protein of interest.

25. A recombinant eukaryotic host cell for manufacturing a protein of interest, wherein the host cell is engineered to overexpress at least one polynucleotide encoding at least one transcription factor, wherein the transcription factor comprises at least:
a) a DNA binding domain comprising:
i) an amino acid sequence as shown in SEQ ID NO: 1, or
ii) a functional homolog of the amino acid sequence as shown in SEQ ID NO: 1 having at least 60% sequence identity to the amino acid sequence as shown in SEQ ID NO: 1 and/or having at least 60% sequence identity to an amino acid sequence as shown in SEQ ID NO: 87, and
b) an activation domain.

26. The recombinant eukaryotic host cell according to claim 25, wherein overexpression of said transcription factor increases the yield of the model proteins scFv (SEQ ID NO. 13) and/or vHH (SEQ ID NO. 14) compared to the host cell prior to engineering.

27. The recombinant eukaryotic host cell according to claim 25, wherein the polynucleotide encoding the at least one transcription factor is integrated in the genome of said host cell or contained in a vector or plasmid, which does not integrate into the genome of said host cell.

28. The recombinant eukaryotic host cell according to claim 25, wherein said polynucleotide encoding at least one transcription factor encodes for a heterologous or homologous transcription factor.

29. The recombinant eukaryotic host cell according to claim 28, wherein the overexpression of the polynucleotide encoding a heterologous transcription factor is achieved by
(i) exchanging or modifying a regulatory sequence operably linked to said polynucleotide encoding the heterologous transcription factor, or
(ii) introducing one or more copies of the polynucleotide encoding the heterologous transcription factor under the control of a promoter into the host cell.

30. The recombinant eukaryotic host cell according to claim 28, wherein the overexpression of the polynucleotide encoding a homologous transcription factor is achieved by
(i) using a promoter which drives expression of said polynucleotide encoding the homologous transcription factor,
(ii) exchanging or modifying a regulatory sequence operably linked to said polynucleotide encoding the homologous transcription factor, or
(iii) introducing one or more copies of the polynucleotide encoding the homologous transcription factor under the control of a promoter into the host cell.

31. The recombinant eukaryotic host cell according to claim 15, wherein the overexpression of the polynucleotide is achieved by
i) exchanging the native promoter of said heterologous and/or homologous transcription factor by a different promoter operably linked to the polynucleotide encoding the homologous transcription factor,
ii) exchanging the native terminator sequence of said heterologous and/or homologous transcription factor by a more efficient terminator sequence,
iii) exchanging the coding sequence of said heterologous and/or homologous transcription factor by a codon-optimized coding sequence, which codon-optimization is done according to the codon-usage of said host cell,
iv) exchanging of a native positive regulatory element of said heterologous and/or homologous transcription factor by a more efficient regulatory element,
v) introducing another positive regulatory element, which is not present in the native expression cassette of said heterologous and/or homologous transcription factor,
vi) deleting a negative regulatory element, which is normally present in the native expression cassette of said heterologous and/or homologous transcription factor, or vii) introducing one or more copies of the polynucleotide encoding a heterologous and/or homologous transcription factor, or a combination thereof.

32. The recombinant eukaryotic host cell according to claim 25, wherein the transcription factor comprises an amino acid sequence as shown in SEQ ID NOs: 15-27.

33. The recombinant eukaryotic host cell according to claim 25, wherein the transcription factor additionally comprises a nuclear localization signal.

34. The recombinant eukaryotic host cell according to claim 33, wherein said nuclear localization signal is a homolog or a heterolog nuclear localization signal.

35. The recombinant eukaryotic host cell according to claim 25, wherein the eukaryotic host cell is a fungal host cell, preferably a fungal host cell, more preferably a yeast host cell selected from the group consisting of Pichia pastoris, Hansenula polymorpha, Trichoderma reesei, Aspergillus niger, Saccharomyces cerevisiae, Kluyveromyces lactis, Yarrowia lipolytica, Pichia methanolica, Candida boidinii, Komagataella spp. and Schizosaccharomyces pombe.

36. The recombinant eukaryotic host cell according to claim 25, wherein the recombinant protein of interest is an enzyme, a therapeutic protein, a food additive or feed additive.

37. The recombinant eukaryotic host cell according to claim 36, wherein the therapeutic protein is an antigen binding protein.

38. The recombinant eukaryotic host cell of claim 25, wherein said host cell is additionally engineered to overexpress at least one polynucleotide encoding at least one ER helper protein.

39. The recombinant eukaryotic host cell according to claim 38, wherein said helper protein has an amino acid sequence as shown in SEQ ID NO: 28 or a functional homolog thereof having at least 70% sequence identity to an amino acid sequence as shown in SEQ ID NO: 28.

40. The recombinant eukaryotic host cell of claim 25, wherein said host cell is additionally engineered to overexpress at least two polynucleotides encoding at least two ER helper proteins.

41. The recombinant eukaryotic host cell according to claim 40, wherein:
a) the first ER helper protein has an amino acid sequence as shown in SEQ ID NO: 28 or a functional homologue thereof having at least 70% sequence identity to the amino acid sequence as shown in SEQ ID NO: 28, and
b) the second ER helper protein has an amino acid sequence:
i) as shown in SEQ ID NO: 37, or a functional homologue thereof having at least 25% sequence identity to the amino acid sequence as shown in SEQ ID NO: 37, or
ii) as shown in SEQ ID NO: 47, or a homologue thereof, wherein the homologue has at least 20% sequence identity to the amino acid sequence as shown in SEQ ID NO: 47, and/or
c) the third ER helper protein has an amino acid sequence:
i) as shown in SEQ ID NO: 55, or a functional homologue thereof having at least 25% sequence identity to the amino acid sequence as shown in SEQ ID NO: 55.

42. The recombinant eukaryotic host cell of claim 25, wherein said host cell is additionally engineered to overexpress at least one polynucleotides encoding one additional transcription factor.

43. The recombinant eukaryotic host cell according to claim 42, wherein the additional transcription factor comprises at least:
a) a DNA binding domain comprising:
i) an amino acid sequence as shown in SEQ ID NO: 65, or
ii) a functional homolog of the amino acid sequence as shown in SEQ ID NO: 65 having at least having at least 50% sequence identity to an amino acid sequence as shown in SEQ ID NO: 65, and
b) an activation domain.

44. The recombinant eukaryotic host cell according to claim 42, wherein the additional transcription factor comprises an amino acid sequence as shown in SEQ ID NOs: 74-82.

45. Use of the recombinant eukaryotic host cell of claim 25 for manufacturing a recombinant protein of interest.
